SEQUENCE LISTING (375 sequences)

SEQ ID NO: 1-2 - skipped (2 entries)

SEQ ID NO: 3 - protein, 10 aa, synthetic construct
  REGION 1..10: note = Hepcidin peptide analogue motif
  VARIANT 1: note = Xaa = Asp, Glu, Iminodiacetic acid, pyroglutamic acid, beta-homoaspartic acid or absent
  VARIANT 4: note = Xaa = Phe or (beta,beta diphenylalanine)
  VARIANT 5: note = Xaa = Pro or beta-homoproline
  VARIANT 8: note = Xaa = Ile, Lys, Glu, Phe, Gln or Arg
  VARIANT 10: note = Xaa = Lys or absent
XTHXXCIXFX

SEQ ID NO: 4 - protein, 10 aa, synthetic construct
  REGION 1..10: note = Hepcidin peptide analogue motif
  VARIANT 1: note = Xaa = Asp, Glu or Iminodiacetic acid
  VARIANT 5: note = Xaa = Pro or beta-homoproline
  VARIANT 8: note = Xaa = Ile Lys or Phe
  VARIANT 10: note = Xaa = absent
XTHFXCIXFX

SEQ ID NO: 5-6 - skipped (2 entries)

SEQ ID NO: 7 - protein, 10 aa, synthetic construct
  REGION 1..10: note = Hepcidin peptide analogue motif
  VARIANT 1: note = Xaa = Gly, Pro or D-Pro
  VARIANT 2: note = Xaa = Pro or Gly
  VARIANT 3: note = Xaa = Arg or Lys
  VARIANT 8: note = Xaa = Val or Thr
  VARIANT 10: note = Xaa = Met, Lys or absent
XXXSKGWXCX

SEQ ID NO: 8 - skipped

SEQ ID NO: 9 - protein, 15 aa, synthetic construct
  REGION 1..15: note = Hepcidin peptide analogue motif
  VARIANT 3: note = Xaa = Gly or absent
  VARIANT 6: note = Xaa = Ser or Pro;
  VARIANT 7: note = Xaa = Ile or Lys
  VARIANT 8: note = Xaa = Gly or absent
  VARIANT 12: note = Xaa = Arg or Ala
  VARIANT 13: note = Xaa = Cys or Val or absent
  VARIANT 14: note = Xaa = Cys, Arg, Thr or absent
  VARIANT 15: note = Xaa = Arg or absent
VCXHRXXXCYRXXXX

SEQ ID NO: 10-14 - skipped (5 entries)

SEQ ID NO: 15 - protein, 10 aa, synthetic construct
  REGION 1..10: note = Hepcidin peptide analogue motif
  VARIANT 1: note = Xaa = Asp, Iminodiacetic acid, pyroglutamic acid, beta-homoaspartic acid or absent
  VARIANT 4: note = Xaa = Phe or (beta,beta diphenylalanine)
  VARIANT 5: note = Xaa = Pro or beta-homoproline
  VARIANT 8: note = Xaa = Ile, Lys, Glu, Phe, Gln or Arg
  VARIANT 10: note = Xaa = Lys or absent
XTHXXCIXFX

SEQ ID NO: 16-17 - skipped (2 entries)

SEQ ID NO: 18 - protein, 10 aa, synthetic construct
  REGION 1..10: note = Hepcidin peptide analogue motif
  VARIANT 1: note = Xaa = Gly, Pro or D-Pro
  VARIANT 2: note = Xaa = Pro or Gly
  VARIANT 3: note = Xaa = Arg or Lys
  VARIANT 8: note = Xaa = Val or Thr
  VARIANT 10: note = Xaa = Met, Lys or absent
XXXSKGWXCX

SEQ ID NO: 19 - skipped

SEQ ID NO: 20 - protein, 15 aa, synthetic construct
  REGION 1..15: note = Hepcidin peptide analogue motif
  VARIANT 3: note = Xaa = Gly or absent
  VARIANT 6: note = Xaa = Ser or Pro;
  VARIANT 7: note = Xaa = Ile or Lys
  VARIANT 8: note = Xaa = Gly or absent
  VARIANT 12: note = Xaa = Arg or Ala
  VARIANT 13: note = Xaa = Cys or Val or absent
  VARIANT 14: note = Xaa = Cys, Arg, Thr or absent
  VARIANT 15: note = Xaa = Arg or absent
VCXHRXXXCYRXXXX

SEQ ID NO: 21-23 - skipped (3 entries)

SEQ ID NO: 24 - protein, 10 aa, synthetic construct
  REGION 1..10: note = Hepcidin peptide analogue motif
  VARIANT 1: note = Xaa = Asp, Iminodiacetic acid, pyroglutamic acid, beta-homoaspartic acid or absent
  VARIANT 4: note = Xaa = Phe or (beta,beta diphenylalanine)
  VARIANT 5: note = Xaa = Pro or beta-homoproline
  VARIANT 8: note = Xaa = Ile, Lys, Glu, Phe, Gln or Arg
  VARIANT 10: note = Xaa = Lys or absent
XTHXXCIXFX

SEQ ID NO: 25 - skipped

SEQ ID NO: 26 - protein, 10 aa, synthetic construct
  REGION 1..10: note = Hepcidin peptide analogue motif
  VARIANT 1: note = Xaa = Gly, Pro or D-Pro
  VARIANT 2: note = Xaa = Pro or Gly
  VARIANT 3: note = Xaa = Arg or Lys
  VARIANT 8: note = Xaa = Val or Thr
  VARIANT 10: note = Xaa = Met, Lys or absent
XXXSKGWXCX

SEQ ID NO: 27 - skipped

SEQ ID NO: 28 - protein, 9 aa, synthetic construct
  REGION 1..9: note = Hepcidin peptide analogue
DTHFPCIIF

SEQ ID NO: 29 - protein, 18 aa, synthetic construct
  REGION 1..18: note = Hepcidin peptide analogue
DTHFPICIFGPRSKGWVC

SEQ ID NO: 30 - protein, 19 aa, synthetic construct
  REGION 1..19: note = Hepcidin peptide analogue
DTHFPCIIFGPRSRGWVCK

SEQ ID NO: 31 - protein, 18 aa, synthetic construct
  REGION 1..18: note = Hepcidin peptide analogue
DTHFPCIIFGPRSKGWVC

SEQ ID NO: 32 - protein, 19 aa, synthetic construct
  REGION 1..19: note = Hepcidin peptide analogue
  MOD_RES 1: note = Xaa is iminodiacetic acid
  MOD_RES 4: note = Xaa is beta,beta diphenylalanine
  MOD_RES 5: note = Xaa is beta-homoproline
XTHXXICIFGPRSKGWVCM

SEQ ID NO: 33 - protein, 19 aa, synthetic construct
  REGION 1..19: note = Hepcidin peptide analogue
DTHFPCIFFGPRSKGWVCK

SEQ ID NO: 34 - protein, 19 aa, synthetic construct
  REGION 1..19: note = Hepcidin peptide analogue
DTHFPCIIFGPRSKGWTCK

SEQ ID NO: 35 - protein, 19 aa, synthetic construct
  REGION 1..19: note = Hepcidin peptide analogue
  MOD_RES 1: note = Xaa is iminodiacetic acid
  MOD_RES 4: note = Xaa is beta,beta diphenylalanine
  MOD_RES 5: note = Xaa is beta-homoproline
XTHXXCIIFGPRSRGWVCK

SEQ ID NO: 36 - protein, 19 aa, synthetic construct
  REGION 1..19: note = Hepcidin peptide analogue
DTHFPCIKFGPRSKGWVCK

SEQ ID NO: 37 - protein, 19 aa, synthetic construct
  REGION 1..19: note = Hepcidin peptide analogue
DTHFPCIQFGPRSKGWVCK

SEQ ID NO: 38 - protein, 19 aa, synthetic construct
  REGION 1..19: note = Hepcidin peptide analogue
DTHFPCIIFGPRSKGWVCK

SEQ ID NO: 39 - protein, 22 aa, synthetic construct
  REGION 1..22: note = Hepcidin peptide analogue
DTHFPICIFVCGHRSICYRRCR

SEQ ID NO: 40 - protein, 23 aa, synthetic construct
  REGION 1..23: note = Hepcidin peptide analogue
ADTHFPICIFVCHRSKGCYRRCR

SEQ ID NO: 41 - protein, 21 aa, synthetic construct
  REGION 1..21: note = Hepcidin peptide analogue
DTHFPICIFVCHRSKGCYRAC

SEQ ID NO: 42 - protein, 19 aa, synthetic construct
  REGION 1..19: note = Hepcidin peptide analogue
DTHFPCIEFGPRSKGWVCK

SEQ ID NO: 43 - protein, 19 aa, synthetic construct
  REGION 1..19: note = Hepcidin peptide analogue
DTHFPICIFGPRAKGWVCM

SEQ ID NO: 44 - protein, 23 aa, synthetic construct
  REGION 1..23: note = Hepcidin peptide analogue
ADTHFPICIFVCHRSKGCYRRCR

SEQ ID NO: 45 - protein, 19 aa, synthetic construct
  REGION 1..19: note = Hepcidin peptide analogue
DTHFPICIFGPRSKGWVCM

SEQ ID NO: 46 - protein, 22 aa, synthetic construct
  REGION 1..22: note = Hepcidin peptide analogue
DTHFPICIFVCHRSKGCYRRCR

SEQ ID NO: 47 - protein, 19 aa, synthetic construct
  REGION 1..19: note = Hepcidin peptide analogue
DTHFPICIFGPRSRGWVCK

SEQ ID NO: 48 - protein, 19 aa, synthetic construct
  REGION 1..19: note = Hepcidin peptide analogue
DTHFPCIIFGPRSKGWVCM

SEQ ID NO: 49 - protein, 19 aa, synthetic construct
  REGION 1..19: note = Hepcidin peptide analogue
DTHFPICIFAPRSKGWVCM

SEQ ID NO: 50 - protein, 20 aa, synthetic construct
  REGION 1..20: note = Hepcidin peptide analogue
DTHFPICIFGPRSKGWVCMH

SEQ ID NO: 51 - protein, 9 aa, synthetic construct
  REGION 1..9: note = Hepcidin peptide analogue
DTHFPCIQF

SEQ ID NO: 52 - protein, 23 aa, synthetic construct
  REGION 1..23: note = Hepcidin peptide analogue
DTHFPICIFVCGHRSKGCYRRCR

SEQ ID NO: 53 - protein, 19 aa, synthetic construct
  REGION 1..19: note = Hepcidin peptide analogue
DTHFAICIFGPRSKGWVCM

SEQ ID NO: 54 - protein, 20 aa, synthetic construct
  REGION 1..20: note = Hepcidin peptide analogue
DTHFPICIFGPHRSKGWVCM

SEQ ID NO: 55 - protein, 19 aa, synthetic construct
  REGION 1..19: note = Hepcidin peptide analogue
DTHFPICIFGPRAKGWVCM

SEQ ID NO: 56 - protein, 19 aa, synthetic construct
  REGION 1..19: note = Hepcidin peptide analogue
DTHFPACIFGPRSKGWVCM

SEQ ID NO: 57 - protein, 23 aa, synthetic construct
  REGION 1..23: note = Hepcidin peptide analogue
DTHFPCIIFVCHRPKGCYRRVCR

SEQ ID NO: 58 - protein, 19 aa, synthetic construct
  REGION 1..19: note = Hepcidin peptide analogue
DTHFPICIFGPRSKAWVCM

SEQ ID NO: 59 - protein, 23 aa, synthetic construct
  REGION 1..23: note = Hepcidin peptide analogue
DTHFPICIFVCGHRGKGCYRRCR

SEQ ID NO: 60 - protein, 19 aa, synthetic construct
  REGION 1..19: note = Hepcidin peptide analogue
ATHFPICIFGPRSKGWVCM

SEQ ID NO: 61 - protein, 19 aa, synthetic construct
  REGION 1..19: note = Hepcidin peptide analogue
DTHFPICIFGPASKGWVCM

SEQ ID NO: 62 - protein, 21 aa, synthetic construct
  REGION 1..21: note = Hepcidin peptide analogue
DTHFPICIFVCHRSKGCYARC

SEQ ID NO: 63 - protein, 19 aa, synthetic construct
  REGION 1..19: note = Hepcidin peptide analogue
DTHFPICIFGPRSKGWVCM

SEQ ID NO: 64 - protein, 19 aa, synthetic construct
  REGION 1..19: note = Hepcidin peptide analogue
DTHFPICIFGPRSAGWVCM

SEQ ID NO: 65 - protein, 19 aa, synthetic construct
  REGION 1..19: note = Hepcidin peptide analogue
DTHAPICIFGPRSKGWVCM

SEQ ID NO: 66 - protein, 21 aa, synthetic construct
  REGION 1..21: note = Hepcidin peptide analogue
DTHFPICIFVCHRSKGCYRRC

SEQ ID NO: 67 - protein, 22 aa, synthetic construct
  REGION 1..22: note = Hepcidin peptide analogue
  MOD_RES 1: note = Xaa is pyroglutamic acid
XTHFPICIFVCHRSKGCYRRCR

SEQ ID NO: 68 - protein, 19 aa, synthetic construct
  REGION 1..19: note = Hepcidin peptide analogue
DTHFPICIFKPRSKGWVCM

SEQ ID NO: 69 - protein, 24 aa, synthetic construct
  REGION 1..24: note = Hepcidin peptide analogue
DTHFPICIFVCGHRSKGCYMRCKT

SEQ ID NO: 70 - protein, 19 aa, synthetic construct
  REGION 1..19: note = Hepcidin peptide analogue
DAHFPICIFGPRSKGWVCM

SEQ ID NO: 71 - protein, 21 aa, synthetic construct
  REGION 1..21: note = Hepcidin peptide analogue
DTHFPICIFVCYRGICYRRCR

SEQ ID NO: 72 - protein, 19 aa, synthetic construct
  REGION 1..19: note = Hepcidin peptide analogue
DTHFPICIFGPRSKGWVCM

SEQ ID NO: 73 - protein, 18 aa, synthetic construct
  REGION 1..18: note = Hepcidin peptide analogue
  MOD_RES 1: note = Xaa is beta-homoaspartic acid
XTHFPICIFGPRSKGWVC

SEQ ID NO: 74 - protein, 19 aa, synthetic construct
  REGION 1..19: note = Hepcidin peptide analogue
DTHFPICIFGPRSKGWACM

SEQ ID NO: 75 - protein, 19 aa, synthetic construct
  REGION 1..19: note = Hepcidin peptide analogue
  MOD_RES 1: note = Xaa is iminodiacetic acid
  MOD_RES 4: note = Xaa is beta,beta diphenylalanine
  MOD_RES 5: note = Xaa is beta-homoproline
  MOD_RES 9: note = Xaa is beta homophenylalanine
XTHXXRCRXGPRSKGWVCM

SEQ ID NO: 76 - protein, 9 aa, synthetic construct
  REGION 1..9: note = Hepcidin peptide analogue
DTHFPCIRF

SEQ ID NO: 77 - protein, 18 aa, synthetic construct
  REGION 1..18: note = Hepcidin peptide analogue
THFPCIIFGPRSKGWVCM

SEQ ID NO: 78 - protein, 9 aa, synthetic construct
  REGION 1..9: note = Hepcidin peptide analogue
DTHFPCIAF

SEQ ID NO: 79 - protein, 9 aa, synthetic construct
  REGION 1..9: note = Hepcidin peptide analogue
DAHFPCIIF

SEQ ID NO: 80 - protein, 22 aa, synthetic construct
  REGION 1..22: note = Hepcidin peptide analogue
DTHFPICIFVCHRPKGCYRRCP

SEQ ID NO: 81 - protein, 19 aa, synthetic construct
  REGION 1..19: note = Hepcidin peptide analogue
DTHFPICIFKPRSKGWVCM

SEQ ID NO: 82 - protein, 10 aa, synthetic construct
  REGION 1..10: note = Hepcidin peptide analogue
DTHFPCIIFK

SEQ ID NO: 83 - protein, 9 aa, synthetic construct
  REGION 1..9: note = Hepcidin peptide analogue
DTHFPCIFF

SEQ ID NO: 84 - protein, 19 aa, synthetic construct
  REGION 1..19: note = Hepcidin peptide analogue
DTHFPICIFGPRSKKWVCM

SEQ ID NO: 85 - protein, 19 aa, synthetic construct
  REGION 1..19: note = Hepcidin peptide analogue
DTHFPICIFGPRSKKWVCM

SEQ ID NO: 86 - protein, 17 aa, synthetic construct
  REGION 1..17: note = Hepcidin peptide analogue
DTHFPICIFCPWGMCCK

SEQ ID NO: 87 - protein, 19 aa, synthetic construct
  REGION 1..19: note = Hepcidin peptide analogue
DTAFPICIFGPRSKGWVCM

SEQ ID NO: 88 - protein, 22 aa, synthetic construct
  REGION 1..22: note = Hepcidin peptide analogue
DTHFPICIFVCYRGICYMRCKT

SEQ ID NO: 89 - protein, 19 aa, synthetic construct
  REGION 1..19: note = Hepcidin peptide analogue
DTHFPICIFGPRSKGAVCM

SEQ ID NO: 90 - protein, 19 aa, synthetic construct
  REGION 1..19: note = Hepcidin peptide analogue
DTHFPICIAGPRSKGWVCM

SEQ ID NO: 91 - protein, 19 aa, synthetic construct
  REGION 1..19: note = Hepcidin peptide analogue
DTHFPICAFGPRSKGWVCM

SEQ ID NO: 92 - protein, 19 aa, synthetic construct
  REGION 1..19: note = Hepcidin peptide analogue
DTHFPIAIFGPRSKGWVAM

SEQ ID NO: 93 - protein, 18 aa, synthetic construct
  REGION 1..18: note = Hepcidin peptide analogue
DTHFPCRRFGPRSKGWVC

SEQ ID NO: 94 - protein, 18 aa, synthetic construct
  REGION 1..18: note = Hepcidin peptide analogue
  MOD_RES 1: note = Xaa is iminodiacetic acid
  MOD_RES 5: note = Xaa is beta-homoproline
  MOD_RES 9: note = Xaa is beta-homophenylalanine
XTHFXCRRXGPRSKGWVC

SEQ ID NO: 95 - skipped

SEQ ID NO: 96 - protein, 22 aa, synthetic construct
  REGION 1..22: note = Hepcidin peptide analogue
DTHFPCIIFVCHRSKGCYWAVC

SEQ ID NO: 97 - protein, 23 aa, synthetic construct
  REGION 1..23: note = Hepcidin peptide analogue
DTHFPCIIFVCHRSKGCYWAVCF

SEQ ID NO: 98 - protein, 24 aa, synthetic construct
  REGION 1..24: note = Hepcidin peptide analogue
DTHFPCIIFVCHRSKGCYWAVCFW

SEQ ID NO: 99 - protein, 19 aa, synthetic construct
  REGION 1..19: note = Hepcidin peptide analogue
DTHFPICIFKPRSKGWVCM

SEQ ID NO: 100 - skipped

SEQ ID NO: 101 - protein, 19 aa, synthetic construct
  REGION 1..19: note = Hepcidin peptide analogue
  MOD_RES 4: note = Xaa is beta,beta diphenylalanine
DTHXPCIIFGPRSRGWVCK

SEQ ID NO: 102 - protein, 19 aa, synthetic construct
  REGION 1..19: note = Hepcidin peptide analogue
  MOD_RES 5: note = Xaa is beta-homoproline
DTHFXCIIFGPRSRGWVCK

SEQ ID NO: 103 - protein, 19 aa, synthetic construct
  REGION 1..19: note = Hepcidin peptide analogue
DTHFPCIIFGPRSRGWRCK

SEQ ID NO: 104 - protein, 19 aa, synthetic construct
  REGION 1..19: note = Hepcidin peptide analogue
DTHFPCIRFGPRSRGWVCK

SEQ ID NO: 105 - protein, 19 aa, synthetic construct
  REGION 1..19: note = Hepcidin peptide analogue
DTHFPCIRFGPRSRGWRCK

SEQ ID NO: 106 - protein, 19 aa, synthetic construct
  REGION 1..19: note = Hepcidin peptide analogue
DTHFPCIIFGPRSRGWVCK

SEQ ID NO: 107 - protein, 18 aa, synthetic construct
  REGION 1..18: note = Hepcidin peptide analogue
DTHFPCIIFGPRSRGVCK

SEQ ID NO: 108 - protein, 19 aa, synthetic construct
  REGION 1..19: note = Hepcidin peptide analogue
DTHFPCIYFGPRSKGWVCK

SEQ ID NO: 109 - protein, 19 aa, synthetic construct
  REGION 1..19: note = Hepcidin peptide analogue
DTHFPCIIFGPRSKGWVCK

SEQ ID NO: 110 - protein, 19 aa, synthetic construct
  REGION 1..19: note = Hepcidin peptide analogue
DTHFPCIIFGPRARGWVCK

SEQ ID NO: 111 - protein, 19 aa, synthetic construct
  REGION 1..19: note = Hepcidin peptide analogue
DTHFPCIIFGPRSRGWVCK

SEQ ID NO: 112 - protein, 19 aa, synthetic construct
  REGION 1..19: note = Hepcidin peptide analogue
DTHFPCIIFGPRSRGWVCK

SEQ ID NO: 113 - protein, 19 aa, synthetic construct
  REGION 1..19: note = Hepcidin peptide analogue
DTHFPICIFKPRSKGWVCK

SEQ ID NO: 114 - protein, 19 aa, synthetic construct
  REGION 1..19: note = Hepcidin peptide analogue
DTHFPCIIFGPRSKGWKCK

SEQ ID NO: 115 - protein, 19 aa, synthetic construct
  REGION 1..19: note = Hepcidin peptide analogue
DTHFPCIKFGPRSKGWKCK

SEQ ID NO: 116 - protein, 19 aa, synthetic construct
  REGION 1..19: note = Hepcidin peptide analogue
DTHFPCLIFGPRSKGWVCK

SEQ ID NO: 117 - protein, 19 aa, synthetic construct
  REGION 1..19: note = Hepcidin peptide analogue
DTHFPCVIFGPRSKGWVCK

SEQ ID NO: 118 - protein, 19 aa, synthetic construct
  REGION 1..19: note = Hepcidin peptide analogue
DTHFPCSIFGPRSKGWVCK

SEQ ID NO: 119 - protein, 19 aa, synthetic construct
  REGION 1..19: note = Hepcidin peptide analogue
DTHFPCQIFGPRSKGWVCK

SEQ ID NO: 120 - protein, 18 aa, synthetic construct
  REGION 1..18: note = Hepcidin peptide analogue
THFPCIIFGPRSKGWVCK

SEQ ID NO: 121 - protein, 18 aa, synthetic construct
  REGION 1..18: note = Hepcidin peptide analogue
THFPCIIFGPRSKGWVCK

SEQ ID NO: 122 - protein, 17 aa, synthetic construct
  REGION 1..17: note = Hepcidin peptide analogue
HFPCIIFGPRSKGWVCK

SEQ ID NO: 123 - protein, 17 aa, synthetic construct
  REGION 1..17: note = Hepcidin peptide analogue
HFPCIIFGPRSKGWVCK

SEQ ID NO: 124 - protein, 19 aa, synthetic construct
  REGION 1..19: note = Hepcidin peptide analogue
DTHFPCISFGPRSKGWVCK

SEQ ID NO: 125 - protein, 19 aa, synthetic construct
  REGION 1..19: note = Hepcidin peptide analogue
DTHFPCIKFGPRSKGWVCK

SEQ ID NO: 126 - protein, 20 aa, synthetic construct
  REGION 1..20: note = Hepcidin peptide analogue
EDTHFPCIIFGPRSKGWVCK

SEQ ID NO: 127 - skipped

SEQ ID NO: 128 - protein, 19 aa, synthetic construct
  REGION 1..19: note = Hepcidin peptide analogue
DTHFPCIIFEPRSKGWVCK

SEQ ID NO: 129 - protein, 19 aa, synthetic construct
  REGION 1..19: note = Hepcidin peptide analogue
DTHFPCIIFSPRSKGWVCK

SEQ ID NO: 130 - protein, 19 aa, synthetic construct
  REGION 1..19: note = Hepcidin peptide analogue
DTHFSCIIFGPRSKGWVCK

SEQ ID NO: 131 - protein, 19 aa, synthetic construct
  REGION 1..19: note = Hepcidin peptide analogue
DTHFPCIIFGPRSRGWVCK

SEQ ID NO: 132 - protein, 19 aa, synthetic construct
  REGION 1..19: note = Hepcidin peptide analogue
DTHFPCIIFGPRSRGWVCK

SEQ ID NO: 133 - protein, 19 aa, synthetic construct
  REGION 1..19: note = Hepcidin peptide analogue
  MOD_RES 1: note = Xaa is iminodiacetic acid
XTHFPCIIFGPRSRGWVCK

SEQ ID NO: 134 - protein, 19 aa, synthetic construct
  REGION 1..19: note = Hepcidin peptide analogue
DTHFPCIIFGPKSKGWVCK

SEQ ID NO: 135 - protein, 19 aa, synthetic construct
  REGION 1..19: note = Hepcidin peptide analogue
DTHFPCIKFGPKSKGWVCK

SEQ ID NO: 136 - protein, 18 aa, synthetic construct
  REGION 1..18: note = Hepcidin peptide analogue
DTHFPCIIFGPRSKGWCK

SEQ ID NO: 137 - protein, 17 aa, synthetic construct
  REGION 1..17: note = Hepcidin peptide analogue
DTHFPCIIFGPRSKGVC

SEQ ID NO: 138 - protein, 17 aa, synthetic construct
  REGION 1..17: note = Hepcidin peptide analogue
DTHFPCIIFGPRSKGCK

SEQ ID NO: 139 - skipped

SEQ ID NO: 140 - protein, 19 aa, synthetic construct
  REGION 1..19: note = Hepcidin peptide analogue
  MOD_RES 7: note = Xaa is 2,3 diaminopropanoic acid
DTHFPCXIFGPRSKGWDCK

SEQ ID NO: 141 - protein, 19 aa, synthetic construct
  REGION 1..19: note = Hepcidin peptide analogue
  MOD_RES 8: note = Xaa is 2,3 diaminopropanoic acid
DTHFPCIXFGPRSKGWDCK

SEQ ID NO: 142 - protein, 19 aa, synthetic construct
  REGION 1..19: note = Hepcidin peptide analogue
  MOD_RES 7: note = Xaa is 2,3 diaminopropanoic acid
DTHFPCXIFGPRSKGWECK

SEQ ID NO: 143 - protein, 19 aa, synthetic construct
  REGION 1..19: note = Hepcidin peptide analogue
  MOD_RES 8: note = Xaa is 2,3 diaminopropanoic acid
DTHFPCIXFGPRSKGWECK

SEQ ID NO: 144 - protein, 19 aa, synthetic construct
  REGION 1..19: note = Hepcidin peptide analogue
DTHFPCIKFGPRSKGWECK

SEQ ID NO: 145 - protein, 19 aa, synthetic construct
  REGION 1..19: note = Hepcidin peptide analogue
DTHFGCIIFGPRSKGWVCK

SEQ ID NO: 146 - protein, 19 aa, synthetic construct
  REGION 1..19: note = Hepcidin peptide analogue
DTHFGCIIFGPRSKGWVCK

SEQ ID NO: 147 - protein, 19 aa, synthetic construct
  REGION 1..19: note = Hepcidin peptide analogue
DTHFRCIIFGPRSKGWVCK

SEQ ID NO: 148 - protein, 19 aa, synthetic construct
  REGION 1..19: note = Hepcidin peptide analogue
DTHFRCIIFGPRSKGWVCK

SEQ ID NO: 149 - protein, 19 aa, synthetic construct
  REGION 1..19: note = Hepcidin peptide analogue
  MOD_RES 5: note = Xaa is sarcosine
DTHFXCIIFGPRSKGWVCK

SEQ ID NO: 150 - protein, 19 aa, synthetic construct
  REGION 1..19: note = Hepcidin peptide analogue
  MOD_RES 5: note = Xaa is sarcosine
DTHFXCIIFGPRSKGWVCK

SEQ ID NO: 151 - protein, 19 aa, synthetic construct
  REGION 1..19: note = Hepcidin peptide analogue
  MOD_RES 5: note = Xaa is beta-alanine
DTHFXCIIFGPRSKGWVCK

SEQ ID NO: 152 - protein, 19 aa, synthetic construct
  REGION 1..19: note = Hepcidin peptide analogue
  MOD_RES 5: note = Xaa is beta-alanine
DTHFXCIIFGPRSKGWVCK

SEQ ID NO: 153 - protein, 19 aa, synthetic construct
  REGION 1..19: note = Hepcidin peptide analogue
DTHFKCIIFGPRSKGWVCK

SEQ ID NO: 154 - protein, 19 aa, synthetic construct
  REGION 1..19: note = Hepcidin peptide analogue
DTHFKCIIFGPRSKGWVCK

SEQ ID NO: 155 - protein, 18 aa, synthetic construct
  REGION 1..18: note = Hepcidin peptide analogue
THFPCIIFGPRSKGWVCM

SEQ ID NO: 156 - protein, 17 aa, synthetic construct
  REGION 1..17: note = Hepcidin peptide analogue
HFPCIIFGPRSKGWVCM

SEQ ID NO: 157 - protein, 17 aa, synthetic construct
  REGION 1..17: note = Hepcidin peptide analogue
HFPCIIFGPRSKGWVCM

SEQ ID NO: 158 - protein, 19 aa, synthetic construct
  REGION 1..19: note = Hepcidin peptide analogue
DTHFPCISFGPRSKGWVCM

SEQ ID NO: 159 - protein, 19 aa, synthetic construct
  REGION 1..19: note = Hepcidin peptide analogue
DTHFPCIKFGPRSKGWVCM

SEQ ID NO: 160 - protein, 20 aa, synthetic construct
  REGION 1..20: note = Hepcidin peptide analogue
EDTHFPCIIFGPRSKGWVCM

SEQ ID NO: 161 - skipped

SEQ ID NO: 162 - protein, 19 aa, synthetic construct
  REGION 1..19: note = Hepcidin peptide analogue
DTHFPCIIFEPRSKGWVCM

SEQ ID NO: 163 - protein, 19 aa, synthetic construct
  REGION 1..19: note = Hepcidin peptide analogue
DTHFKCIEFGPRSKGWVCK

SEQ ID NO: 164 - protein, 19 aa, synthetic construct
  REGION 1..19: note = Hepcidin peptide analogue
DTHFPCIIFGPRSKGWACK

SEQ ID NO: 165 - protein, 19 aa, synthetic construct
  REGION 1..19: note = Hepcidin peptide analogue
DTHFPCIIFEPRSKGWVCK

SEQ ID NO: 166 - protein, 22 aa, synthetic construct
  REGION 1..22: note = Hepcidin peptide analogue
DTHFPCIIFGPRSKGWVCKKKK

SEQ ID NO: 167 - protein, 22 aa, synthetic construct
  REGION 1..22: note = Hepcidin peptide analogue
DTHFPCIIFEPRSKGWVCKKKK

SEQ ID NO: 168 - protein, 20 aa, synthetic construct
  REGION 1..20: note = Hepcidin peptide analogue
DTHFPCIIFGPRSKGWVCKK

SEQ ID NO: 169 - protein, 19 aa, synthetic construct
  REGION 1..19: note = Hepcidin peptide analogue
DTAFPCIIFGPRSKGWVCK

SEQ ID NO: 170 - protein, 19 aa, synthetic construct
  REGION 1..19: note = Hepcidin peptide analogue
DTKFPCIIFGPRSKGWVCK

SEQ ID NO: 171 - protein, 23 aa, synthetic construct
  REGION 1..23: note = Hepcidin peptide analogue
DTHFPCIIFVCHRPKGCYRRVCR

SEQ ID NO: 172 - protein, 19 aa, synthetic construct
  REGION 1..19: note = Hepcidin peptide analogue
DTHFPCIKFGPRSKGWVCK

SEQ ID NO: 173 - protein, 19 aa, synthetic construct
  REGION 1..19: note = Hepcidin peptide analogue
DTHFPCIKFKPRSKGWVCK

SEQ ID NO: 174 - protein, 19 aa, synthetic construct
  REGION 1..19: note = Hepcidin peptide analogue
DTHFPCIKFGPKSKGWVCK

SEQ ID NO: 175 - protein, 19 aa, synthetic construct
  REGION 1..19: note = Hepcidin peptide analogue
DTHFPCIKFGPRSKGWVCK

SEQ ID NO: 176 - protein, 19 aa, synthetic construct
  REGION 1..19: note = Hepcidin peptide analogue
DTHFPCIKFGPRSKGWVCK

SEQ ID NO: 177 - protein, 19 aa, synthetic construct
  REGION 1..19: note = Hepcidin peptide analogue
DTHFPCIKFGPRSKGWTCK

SEQ ID NO: 178 - protein, 19 aa, synthetic construct
  REGION 1..19: note = Hepcidin peptide analogue
DTHFPCIEFGPRSKGWTCK

SEQ ID NO: 179 - protein, 18 aa, synthetic construct
  REGION 1..18: note = Hepcidin peptide analogue
DTHFPICIFGPRSKGWVC

SEQ ID NO: 180 - protein, 19 aa, synthetic construct
  REGION 1..19: note = Hepcidin peptide analogue
DTHFPCIKFGPRSKGWVCK

SEQ ID NO: 181 - protein, 19 aa, synthetic construct
  REGION 1..19: note = Hepcidin peptide analogue
DTHFPCIKFGPRSKGWVCK

SEQ ID NO: 182 - protein, 19 aa, synthetic construct
  REGION 1..19: note = Hepcidin peptide analogue
DTHFPCIKFGPRSKGWVCK

SEQ ID NO: 183 - protein, 19 aa, synthetic construct
  REGION 1..19: note = Hepcidin peptide analogue
DTHFPCIKFGPRSKGWVCK

SEQ ID NO: 184 - protein, 19 aa, synthetic construct
  REGION 1..19: note = Hepcidin peptide analogue
DTHFPCIKFGPRSKGWVCK

SEQ ID NO: 185 - protein, 19 aa, synthetic construct
  REGION 1..19: note = Hepcidin peptide analogue
DTHFPCIKFGPRSKGWVCK

SEQ ID NO: 186 - protein, 19 aa, synthetic construct
  REGION 1..19: note = Hepcidin peptide analogue
DTHFPCIKFGPRSKGWVCK

SEQ ID NO: 187 - protein, 20 aa, synthetic construct
  REGION 1..20: note = Hepcidin peptide analogue
VDTHFPCIKFGPRSKGWVCK

SEQ ID NO: 188 - protein, 20 aa, synthetic construct
  REGION 1..20: note = Hepcidin peptide analogue
LDTHFPCIKFGPRSKGWVCK

SEQ ID NO: 189 - protein, 19 aa, synthetic construct
  REGION 1..19: note = Hepcidin peptide analogue
DTHFPCIKFGPRSKGWVCK

SEQ ID NO: 190 - protein, 19 aa, synthetic construct
  REGION 1..19: note = Hepcidin peptide analogue
DTHFPCIKFGPRSKGWVCK

SEQ ID NO: 191 - protein, 19 aa, synthetic construct
  REGION 1..19: note = Hepcidin peptide analogue
DTHFPCIKFGPRSKGWVCK

SEQ ID NO: 192 - protein, 19 aa, synthetic construct
  REGION 1..19: note = Hepcidin peptide analogue
DTHFPCIKFGPRSKGWVCK

SEQ ID NO: 193 - protein, 19 aa, synthetic construct
  REGION 1..19: note = Hepcidin peptide analogue
DTHFPCIKFGPRSKGWVCK

SEQ ID NO: 194 - protein, 19 aa, synthetic construct
  REGION 1..19: note = Hepcidin peptide analogue
DTHFPCIIFGPRSKGWKCK

SEQ ID NO: 195 - protein, 19 aa, synthetic construct
  REGION 1..19: note = Hepcidin peptide analogue
DTHFPCIIFGPRSKGWECK

SEQ ID NO: 196 - protein, 19 aa, synthetic construct
  REGION 1..19: note = Hepcidin peptide analogue
DTHFPCRRFGPRSKGWVCK

SEQ ID NO: 197-198 - skipped (2 entries)

SEQ ID NO: 199 - protein, 18 aa, synthetic construct
  REGION 1..18: note = Hepcidin peptide analogue
DTHFPICIFGPRSKGWVC

SEQ ID NO: 200 - protein, 18 aa, synthetic construct
  REGION 1..18: note = Hepcidin peptide analogue
DTHFPICIFGPRSKGWVC

SEQ ID NO: 201 - protein, 19 aa, synthetic construct
  REGION 1..19: note = Hepcidin peptide analogue
DTHFPCIIFGPRSRGWVCK

SEQ ID NO: 202 - protein, 19 aa, synthetic construct
  REGION 1..19: note = Hepcidin peptide analogue
DTHFPCIIFGPRSRGWVCK

SEQ ID NO: 203 - protein, 19 aa, synthetic construct
  REGION 1..19: note = Hepcidin peptide analogue
DTHFPCIIFGPRSRGWVCK

SEQ ID NO: 204 - protein, 19 aa, synthetic construct
  REGION 1..19: note = Hepcidin peptide analogue
DTHFPCIKFEPRSKGWVCK

SEQ ID NO: 205 - protein, 19 aa, synthetic construct
  REGION 1..19: note = Hepcidin peptide analogue
DTHFPCIKFEPRSKGWTCK

SEQ ID NO: 206 - protein, 18 aa, synthetic construct
  REGION 1..18: note = Hepcidin peptide analogue
DTHFPCIKFEPRSKGWCK

SEQ ID NO: 207 - protein, 17 aa, synthetic construct
  REGION 1..17: note = Hepcidin peptide analogue
DTHFPCIKFEPRSKGCK

SEQ ID NO: 208 - protein, 16 aa, synthetic construct
  REGION 1..16: note = Hepcidin peptide analogue
DTHFPCIFEPRSKGCK

SEQ ID NO: 209 - protein, 17 aa, synthetic construct
  REGION 1..17: note = Hepcidin peptide analogue
DTHFPCIFEPRSKGWCK

SEQ ID NO: 210 - protein, 16 aa, synthetic construct
  REGION 1..16: note = Hepcidin peptide analogue
DTHFPCIKFGPRSKCK

SEQ ID NO: 211 - protein, 15 aa, synthetic construct
  REGION 1..15: note = Hepcidin peptide analogue
DTHFPCIKFGPRSCK

SEQ ID NO: 212 - protein, 14 aa, synthetic construct
  REGION 1..14: note = Hepcidin peptide analogue
DTHFPCIKFGPRCK

SEQ ID NO: 213 - protein, 13 aa, synthetic construct
  REGION 1..13: note = Hepcidin peptide analogue
DTHFPCIKFGPCK

SEQ ID NO: 214 - protein, 12 aa, synthetic construct
  REGION 1..12: note = Hepcidin peptide analogue
DTHFPCIKFGCK

SEQ ID NO: 215 - protein, 11 aa, synthetic construct
  REGION 1..11: note = Hepcidin peptide analogue
DTHFPCIKFCK

SEQ ID NO: 216 - protein, 10 aa, synthetic construct
  REGION 1..10: note = Hepcidin peptide analogue
DTHFPCIKFC

SEQ ID NO: 217 - protein, 19 aa, synthetic construct
  REGION 1..19: note = Hepcidin peptide analogue
DTHFPCIKFGPRSKGWVCK

SEQ ID NO: 218 - protein, 20 aa, synthetic construct
  REGION 1..20: note = Hepcidin peptide analogue
DTHFPCIKFKAPRSKGWVCK

SEQ ID NO: 219 - protein, 20 aa, synthetic construct
  REGION 1..20: note = Hepcidin peptide analogue
DTHFPCIKFGPKASKGWVCK

SEQ ID NO: 220 - protein, 20 aa, synthetic construct
  REGION 1..20: note = Hepcidin peptide analogue
DTHFPCIKFGPRSKAGWVCK

SEQ ID NO: 221 - protein, 20 aa, synthetic construct
  REGION 1..20: note = Hepcidin peptide analogue
DTHFPCIKFGPRSKGWVCKA

SEQ ID NO: 222 - protein, 19 aa, synthetic construct
  REGION 1..19: note = Hepcidin peptide analogue
DTHFPCIKFGPRSKGWVCK

SEQ ID NO: 223 - protein, 19 aa, synthetic construct
  REGION 1..19: note = Hepcidin peptide analogue
DTHFPCIKFKPRSKGWVCK

SEQ ID NO: 224 - protein, 19 aa, synthetic construct
  REGION 1..19: note = Hepcidin peptide analogue
DTHFPCIKFGPKSKGWVCK

SEQ ID NO: 225 - protein, 19 aa, synthetic construct
  REGION 1..19: note = Hepcidin peptide analogue
DTHFPCIKFGPRSKGWVCK

SEQ ID NO: 226 - protein, 19 aa, synthetic construct
  REGION 1..19: note = Hepcidin peptide analogue
DTHFPCIKFGPRSKGWVCK

SEQ ID NO: 227 - protein, 19 aa, synthetic construct
  REGION 1..19: note = Hepcidin peptide analogue
DTHFPCIKFGPRSKGWVCK

SEQ ID NO: 228 - protein, 19 aa, synthetic construct
  REGION 1..19: note = Hepcidin peptide analogue
DTHFPCIKFKPRSKGWVCK

SEQ ID NO: 229 - protein, 19 aa, synthetic construct
  REGION 1..19: note = Hepcidin peptide analogue
DTHFPCIKFGPKSKGWVCK

SEQ ID NO: 230 - protein, 19 aa, synthetic construct
  REGION 1..19: note = Hepcidin peptide analogue
DTHFPCIKFGPRSKGWVCK

SEQ ID NO: 231 - protein, 19 aa, synthetic construct
  REGION 1..19: note = Hepcidin peptide analogue
DTHFPCIKFGPRSKGWVCK

SEQ ID NO: 232 - protein, 19 aa, synthetic construct
  REGION 1..19: note = Hepcidin peptide analogue
DTHFPCIKFGPRSKGWVCK

SEQ ID NO: 233 - protein, 19 aa, synthetic construct
  REGION 1..19: note = Hepcidin peptide analogue
DTHFPCIKFKPRSKGWVCK

SEQ ID NO: 234 - protein, 19 aa, synthetic construct
  REGION 1..19: note = Hepcidin peptide analogue
DTHFPCIKFGPKSKGWVCK

SEQ ID NO: 235 - protein, 19 aa, synthetic construct
  REGION 1..19: note = Hepcidin peptide analogue
DTHFPCIKFGPRSKGWVCK

SEQ ID NO: 236 - protein, 19 aa, synthetic construct
  REGION 1..19: note = Hepcidin peptide analogue
DTHFPCIKFGPRSKGWVCK

SEQ ID NO: 237 - protein, 19 aa, synthetic construct
  REGION 1..19: note = Hepcidin peptide analogue
DTHFPCIKFEPRSKKWVCK

SEQ ID NO: 238 - protein, 19 aa, synthetic construct
  REGION 1..19: note = Hepcidin peptide analogue
DTHFPCIKFGPRSKGWVCK

SEQ ID NO: 239 - protein, 19 aa, synthetic construct
  REGION 1..19: note = Hepcidin peptide analogue
DTHFPCIKFGPRSKGWVCK

SEQ ID NO: 240 - protein, 19 aa, synthetic construct
  REGION 1..19: note = Hepcidin peptide analogue
DTHFPCIKFGPRSKGWVCK

SEQ ID NO: 241 - protein, 17 aa, synthetic construct
  REGION 1..17: note = Hepcidin peptide analogue
DTHFPCIKFEPRSKGCK

SEQ ID NO: 242 - protein, 17 aa, synthetic construct
  REGION 1..17: note = Hepcidin peptide analogue
DTHFPCIKFKPRSKGCK

SEQ ID NO: 243 - protein, 17 aa, synthetic construct
  REGION 1..17: note = Hepcidin peptide analogue
DTHFPCIKFEPKSKGCK

SEQ ID NO: 244 - protein, 17 aa, synthetic construct
  REGION 1..17: note = Hepcidin peptide analogue
DTHFPCIKFEPRSKGCK

SEQ ID NO: 245 - protein, 17 aa, synthetic construct
  REGION 1..17: note = Hepcidin peptide analogue
DTHFPCIKFEPRSKKCK

SEQ ID NO: 246 - protein, 17 aa, synthetic construct
  REGION 1..17: note = Hepcidin peptide analogue
DTHFPCIKFEPRSKGCK

SEQ ID NO: 247 - protein, 18 aa, synthetic construct
  REGION 1..18: note = Hepcidin peptide analogue
DTHFPCIKFEPRSKGCKK

SEQ ID NO: 248 - protein, 17 aa, synthetic construct
  REGION 1..17: note = Hepcidin peptide analogue
DTHFPCIKFEPRSKGCK

SEQ ID NO: 249 - protein, 17 aa, synthetic construct
  REGION 1..17: note = Hepcidin peptide analogue
DTHFPCIKFKPRSKGCK

SEQ ID NO: 250 - protein, 17 aa, synthetic construct
  REGION 1..17: note = Hepcidin peptide analogue
DTHFPCIKFEPKSKGCK

SEQ ID NO: 251 - protein, 17 aa, synthetic construct
  REGION 1..17: note = Hepcidin peptide analogue
DTHFPCIKFEPRSKGCK

SEQ ID NO: 252 - protein, 17 aa, synthetic construct
  REGION 1..17: note = Hepcidin peptide analogue
DTHFPCIKFEPRSKKCK

SEQ ID NO: 253 - protein, 18 aa, synthetic construct
  REGION 1..18: note = Hepcidin peptide analogue
DTHFPCIKFEPRSKGCKK

SEQ ID NO: 254 - protein, 18 aa, synthetic construct
  REGION 1..18: note = Hepcidin peptide analogue
DTHFPCIKFEPRSKGCKK

SEQ ID NO: 255 - protein, 19 aa, synthetic construct
  REGION 1..19: note = Hepcidin peptide analogue
DTHFPCIKFGPRSKGWVCK

SEQ ID NO: 256 - protein, 19 aa, synthetic construct
  REGION 1..19: note = Hepcidin peptide analogue
AAHFPCIKFGPRSKGWVCK

SEQ ID NO: 257 - protein, 19 aa, synthetic construct
  REGION 1..19: note = Hepcidin peptide analogue
ATHFPCIKFGPRSKGWVCK

SEQ ID NO: 258 - protein, 19 aa, synthetic construct
  REGION 1..19: note = Hepcidin peptide analogue
DAHFPCIKFGPRSKGWVCK

SEQ ID NO: 259 - protein, 19 aa, synthetic construct
  REGION 1..19: note = Hepcidin peptide analogue
DTHAPCIKFGPRSKGWVCK

SEQ ID NO: 260 - protein, 19 aa, synthetic construct
  REGION 1..19: note = Hepcidin peptide analogue
DTHFPCIKAGPRSKGWVCK

SEQ ID NO: 261 - protein, 19 aa, synthetic construct
  REGION 1..19: note = Hepcidin peptide analogue
DTHFPCIKFEPRSKGWVCK

SEQ ID NO: 262 - protein, 19 aa, synthetic construct
  REGION 1..19: note = Hepcidin peptide analogue
DTHFPCIKFEPRSKGWECK

SEQ ID NO: 263 - protein, 18 aa, synthetic construct
  REGION 1..18: note = Hepcidin peptide analogue
DTHFPCIIFEPRSKGWEC

SEQ ID NO: 264 - protein, 19 aa, synthetic construct
  REGION 1..19: note = Hepcidin peptide analogue
DTHFPCIKFKPRSKGWECK

SEQ ID NO: 265 - protein, 19 aa, synthetic construct
  REGION 1..19: note = Hepcidin peptide analogue
DTHFPCIKFEPKSKGWECK

SEQ ID NO: 266 - protein, 19 aa, synthetic construct
  REGION 1..19: note = Hepcidin peptide analogue
DTHAPCIKFEPRSKGWECK

SEQ ID NO: 267 - protein, 17 aa, synthetic construct
  REGION 1..17: note = Hepcidin peptide analogue
  MOD_RES 1: note = Xaa is iminodiacetic acid
XTHFPCIKFEPRSKKCK

SEQ ID NO: 268 - protein, 18 aa, synthetic construct
  REGION 1..18: note = Hepcidin peptide analogue
DTHFPCIKFEPRSKGWEC

SEQ ID NO: 269 - protein, 19 aa, synthetic construct
  REGION 1..19: note = Hepcidin peptide analogue
DTHFPCIKFGPRSKGWVCK

SEQ ID NO: 270 - protein, 18 aa, synthetic construct
  REGION 1..18: note = Hepcidin peptide analogue
DTHFPCIKFKPRSKGWVC

SEQ ID NO: 271 - protein, 18 aa, synthetic construct
  REGION 1..18: note = Hepcidin peptide analogue
DTHFPCIKFKPRSKGWVC

SEQ ID NO: 272 - protein, 18 aa, synthetic construct
  REGION 1..18: note = Hepcidin peptide analogue
DTHFPCIKFKPRSKGWVC

SEQ ID NO: 273 - protein, 18 aa, synthetic construct
  REGION 1..18: note = Hepcidin peptide analogue
DTHFPCIKFKPRSKGWVC

SEQ ID NO: 274 - protein, 18 aa, synthetic construct
  REGION 1..18: note = Hepcidin peptide analogue
DTHFPCIKFKPRSKGWVC

SEQ ID NO: 275 - protein, 18 aa, synthetic construct
  REGION 1..18: note = Hepcidin peptide analogue
DTHFPCIKFKPRSKGWVC

SEQ ID NO: 276 - protein, 18 aa, synthetic construct
  REGION 1..18: note = Hepcidin peptide analogue
DTHFPCIKFKPRSKGWVC

SEQ ID NO: 277 - protein, 19 aa, synthetic construct
  REGION 1..19: note = Hepcidin peptide analogue
  MOD_RES 1: note = Xaa is iminodiacetic acid
XTHFPCIKFEPRSKGWVCK

SEQ ID NO: 278 - protein, 19 aa, synthetic construct
  REGION 1..19: note = Hepcidin peptide analogue
  MOD_RES 1: note = Xaa is iminodiacetic acid
XTHFPCIKFEPRSKGWVCK

SEQ ID NO: 279 - protein, 19 aa, synthetic construct
  REGION 1..19: note = Hepcidin peptide analogue
  MOD_RES 1: note = Xaa is iminodiacetic acid
XTHFPCIKFEPRSKGWVCK

SEQ ID NO: 280 - protein, 19 aa, synthetic construct
  REGION 1..19: note = Hepcidin peptide analogue
  MOD_RES 1: note = Xaa is iminodiacetic acid
XTHFPCIKFEPRSKGWVCK

SEQ ID NO: 281 - protein, 19 aa, synthetic construct
  REGION 1..19: note = Hepcidin peptide analogue
  MOD_RES 1: note = Xaa is iminodiacetic acid
XTHFPCIKFEPRSKGWVCK

SEQ ID NO: 282 - protein, 19 aa, synthetic construct
  REGION 1..19: note = Hepcidin peptide analogue
  MOD_RES 1: note = Xaa is iminodiacetic acid
XTHFPCIKFEPRSKGWVCK

SEQ ID NO: 283 - protein, 19 aa, synthetic construct
  REGION 1..19: note = Hepcidin peptide analogue
  MOD_RES 1: note = Xaa is iminodiacetic acid
XTHFPCIKFEPRSKGWVCK

SEQ ID NO: 284 - protein, 17 aa, synthetic construct
  REGION 1..17: note = Hepcidin peptide analogue
HFPCIKFEPRSKGWVCK

SEQ ID NO: 285 - protein, 17 aa, synthetic construct
  REGION 1..17: note = Hepcidin peptide analogue
HFPCIKFEPRSKGWVCK

SEQ ID NO: 286 - protein, 17 aa, synthetic construct
  REGION 1..17: note = Hepcidin peptide analogue
DTHFPCIKFEPHSKGCK

SEQ ID NO: 287 - protein, 16 aa, synthetic construct
  REGION 1..16: note = Hepcidin peptide analogue
DTHFPCIHFEPHSKGC

SEQ ID NO: 288 - protein, 17 aa, synthetic construct
  REGION 1..17: note = Hepcidin peptide analogue
DTHFPCIKFEPHSKGCK

SEQ ID NO: 289 - protein, 16 aa, synthetic construct
  REGION 1..16: note = Hepcidin peptide analogue
DTHFPCIKFEPREKEC

SEQ ID NO: 290 - protein, 16 aa, synthetic construct
  REGION 1..16: note = Hepcidin peptide analogue
DTAFPCIKFEPRSKEC

SEQ ID NO: 291 - protein, 12 aa, synthetic construct
  REGION 1..12: note = Hepcidin peptide analogue
DTHFPCIKFECK

SEQ ID NO: 292 - protein, 15 aa, synthetic construct
  REGION 1..15: note = Hepcidin peptide analogue
DTHFPIAIFAAGICI

SEQ ID NO: 293 - protein, 14 aa, synthetic construct
  REGION 1..14: note = Hepcidin peptide analogue
DTHFPIAIFAAICI

SEQ ID NO: 294 - protein, 13 aa, synthetic construct
  REGION 1..13: note = Hepcidin peptide analogue
DTHFPIAIFAICI

SEQ ID NO: 295 - protein, 12 aa, synthetic construct
  REGION 1..12: note = Hepcidin peptide analogue
DTHFPIAIFICI

SEQ ID NO: 296 - protein, 11 aa, synthetic construct
  REGION 1..11: note = Hepcidin peptide analogue
DTHFPIAIICI

SEQ ID NO: 297 - protein, 10 aa, synthetic construct
  REGION 1..10: note = Hepcidin peptide analogue
DTHFPIAICI

SEQ ID NO: 298 - protein, 9 aa, synthetic construct
  REGION 1..9: note = Hepcidin peptide analogue
DTHFPIICI

SEQ ID NO: 299 - protein, 9 aa, synthetic construct
  REGION 1..9: note = Hepcidin peptide analogue
DTHICIAIF

SEQ ID NO: 300 - protein, 9 aa, synthetic construct
  REGION 1..9: note = Hepcidin peptide analogue
DTHCPIAIF

SEQ ID NO: 301 - protein, 9 aa, synthetic construct
  REGION 1..9: note = Hepcidin peptide analogue
DTHFPCIIA

SEQ ID NO: 302 - protein, 9 aa, synthetic construct
  REGION 1..9: note = Hepcidin peptide analogue
DTHFPCAIF

SEQ ID NO: 303 - protein, 9 aa, synthetic construct
  REGION 1..9: note = Hepcidin peptide analogue
DTHFACIIF

SEQ ID NO: 304 - protein, 9 aa, synthetic construct
  REGION 1..9: note = Hepcidin peptide analogue
DTHFACIIF

SEQ ID NO: 305 - protein, 9 aa, synthetic construct
  REGION 1..9: note = Hepcidin peptide analogue
DTHAPCIIF

SEQ ID NO: 306 - protein, 9 aa, synthetic construct
  REGION 1..9: note = Hepcidin peptide analogue
DTAFPCIIF

SEQ ID NO: 307 - protein, 9 aa, synthetic construct
  REGION 1..9: note = Hepcidin peptide analogue
ATHFPCIIF

SEQ ID NO: 308 - protein, 9 aa, synthetic construct
  REGION 1..9: note = Hepcidin peptide analogue
  MOD_RES 1: note = Xaa is iminodiacetic acid
  MOD_RES 5: note = Xaa is beta-homoproline
XTHFXCIIF

SEQ ID NO: 309 - skipped

SEQ ID NO: 310 - protein, 9 aa, synthetic construct
  REGION 1..9: note = Hepcidin peptide analogue
DTHFPCIEF

SEQ ID NO: 311 - protein, 9 aa, synthetic construct
  REGION 1..9: note = Hepcidin peptide analogue
DTHFPCIIF

SEQ ID NO: 312 - protein, 9 aa, synthetic construct
  REGION 1..9: note = Hepcidin peptide analogue
DTHFPAIIF

SEQ ID NO: 313 - protein, 9 aa, synthetic construct
  REGION 1..9: note = Hepcidin peptide analogue
DTHFPSIIF

SEQ ID NO: 314 - protein, 9 aa, synthetic construct
  REGION 1..9: note = Hepcidin peptide analogue
DTHFPCIKF

SEQ ID NO: 315 - skipped

SEQ ID NO: 316 - protein, 8 aa, synthetic construct
  REGION 1..8: note = Hepcidin peptide analogue
DTHFPCIF

SEQ ID NO: 317-319 - skipped (3 entries)

SEQ ID NO: 320 - protein, 10 aa, synthetic construct
  REGION 1..10: note = Hepcidin peptide analogue
DTHFPCIKFF

SEQ ID NO: 321 - protein, 9 aa, synthetic construct
  REGION 1..9: note = Hepcidin peptide analogue
YTHFPCIIF

SEQ ID NO: 322 - protein, 9 aa, synthetic construct
  REGION 1..9: note = Hepcidin peptide analogue
LTHFPCIIF

SEQ ID NO: 323 - protein, 9 aa, synthetic construct
  REGION 1..9: note = Hepcidin peptide analogue
ETHFPCIIF

SEQ ID NO: 324 - protein, 9 aa, synthetic construct
  REGION 1..9: note = Hepcidin peptide analogue
DRHFPCIIF

SEQ ID NO: 325 - protein, 9 aa, synthetic construct
  REGION 1..9: note = Hepcidin peptide analogue
DTKFPCIIF

SEQ ID NO: 326 - protein, 9 aa, synthetic construct
  REGION 1..9: note = Hepcidin peptide analogue
DTHFECIIF

SEQ ID NO: 327 - protein, 9 aa, synthetic construct
  REGION 1..9: note = Hepcidin peptide analogue
DTHFPCIIK

SEQ ID NO: 328 - protein, 9 aa, synthetic construct
  REGION 1..9: note = Hepcidin peptide analogue
DTHFPCIIR

SEQ ID NO: 329 - protein, 9 aa, synthetic construct
  REGION 1..9: note = Hepcidin peptide analogue
DTHFPCIEF

SEQ ID NO: 330 - protein, 9 aa, synthetic construct
  REGION 1..9: note = Hepcidin peptide analogue
DTHFPCIVF

SEQ ID NO: 331 - protein, 9 aa, synthetic construct
  REGION 1..9: note = Hepcidin peptide analogue
DTHFPCILF

SEQ ID NO: 332 - protein, 9 aa, synthetic construct
  REGION 1..9: note = Hepcidin peptide analogue
DTHFPCILK

SEQ ID NO: 333 - protein, 9 aa, synthetic construct
  REGION 1..9: note = Hepcidin peptide analogue
DTHFPCIEK

SEQ ID NO: 334 - protein, 19 aa, synthetic construct
  REGION 1..19: note = Hepcidine peptide analogue
DTHFPICIFGARSKGWVMC

SEQ ID NO: 335 - protein, 25 aa, Homo sapiens
DTHFPICIFCCGCCHRSKCGMCCKT

SEQ ID NO: 336 - protein, 9 aa, Homo sapiens
DTHFPICIF

SEQ ID NO: 337 - protein, 9 aa, synthetic construct
  REGION 1..9: note = Retro inverse of mini-hepcidin peptide
  SITE 1..9: note = All amino acids are the D stereo isoform
FICIPFHTD

SEQ ID NO: 338 - protein, 9 aa, synthetic construct
  REGION 1..9: note = Dimerized peptide
  DISULFID 6: note = dimer linked through disulphide linkage
DTHFPCIIF

SEQ ID NO: 339 - protein, 8 aa, synthetic construct
  REGION 1..8: note = Dimerized peptide
  DISULFID 6: note = dimer linked through disulphide linkage
DTHFPCIF

SEQ ID NO: 340 - skipped

SEQ ID NO: 341 - protein, 10 aa, synthetic construct
  REGION 1..10: note = Dimerized peptide
  DISULFID 6: note = dimer linked through disulphide linkage
DTHFPCIKFF

SEQ ID NO: 342 - protein, 9 aa, synthetic construct
  REGION 1..9: note = Dimerized peptide
  DISULFID 6: note = dimer linked through disulphide linkage
LTHFPCIIF

SEQ ID NO: 343 - protein, 9 aa, synthetic construct
  REGION 1..9: note = Dimerized peptide
  DISULFID 6: note = dimer linked through disulphide linkage
ETHFPCIIF

SEQ ID NO: 344 - protein, 9 aa, synthetic construct
  REGION 1..9: note = Dimerized peptide
  DISULFID 6: note = dimer linked through disulphide linkage
DTKFPCIIF

SEQ ID NO: 345 - protein, 9 aa, synthetic construct
  REGION 1..9: note = Dimerized peptide
  DISULFID 6: note = dimer linked through disulphide linkage
DTHFPCIIK

SEQ ID NO: 346 - protein, 9 aa, synthetic construct
  REGION 1..9: note = Dimerized peptide
  DISULFID 6: note = dimer linked through disulphide linkage
DTHFPCIIR

SEQ ID NO: 347 - protein, 9 aa, synthetic construct
  REGION 1..9: note = Dimerized peptide
  DISULFID 6: note = dimer linked through disulphide linkage
DTHFPCIVF

SEQ ID NO: 348 - protein, 9 aa, synthetic construct
  REGION 1..9: note = Dimerized peptide
  DISULFID 6: note = dimer linked through disulphide linkage
DTHFPCILF

SEQ ID NO: 349 - protein, 9 aa, synthetic construct
  REGION 1..9: note = Dimerized peptide
  DISULFID 6: note = dimer linked through disulphide linkage
DTHFPCILK

SEQ ID NO: 350 - protein, 9 aa, synthetic construct
  REGION 1..9: note = Dimerized peptide
  DISULFID 6: note = dimer linked through disulphide linkage
DTHFPCIEK

SEQ ID NO: 351 - protein, 9 aa, synthetic construct
  REGION 1..9: note = Dimerized peptide
  DISULFID 6: note = dimer linked through disulphide linkage
DTHFPCIIF

SEQ ID NO: 352 - protein, 9 aa, synthetic construct
  REGION 1..9: note = Dimerized peptide
  DISULFID 6: note = dimer linked through disulphide linkage
DTHFPCIKF

SEQ ID NO: 353 - protein, 9 aa, synthetic construct
  REGION 1..9: note = Dimerized peptide
  DISULFID 6: note = dimer linked through disulphide linkage
DTHFPCIKF

SEQ ID NO: 354 - protein, 9 aa, synthetic construct
  REGION 1..9: note = Hepcidin dimerized peptide
  SITE 1: note = CROSSLINK - Diethylene glycol linkage between Asp residues of dimer
  DISULFID 6: note = Disulphide linkage between Cys residues of dimer
DTHFPCIKF

SEQ ID NO: 355 - protein, 9 aa, synthetic construct
  REGION 1..9: note = Hepcidin dimerized peptide
  DISULFID 6: note = disulphide linkage between Cys residues of dimer
DTKFPCIRF

SEQ ID NO: 356 - protein, 9 aa, synthetic construct
  REGION 1..9: note = Hepcidin dimerized peptide
  SITE 3: note = CROSSLINK - Diethylene glycol linkage between Lys residues of dimer
  DISULFID 6: note = disulphide linkage between Cys residues of dimer
DTKFPCIRF

SEQ ID NO: 357 - protein, 9 aa, synthetic construct
  REGION 1..9: note = Hepcidin dimerized peptide
  DISULFID 6: note = disulphide linkage between Cys residues of dimer
DTHFPCIKF

SEQ ID NO: 358 - protein, 9 aa, synthetic construct
  REGION 1..9: note = Hepcidin dimerized peptide
  DISULFID 6: note = disulphide linkage between Cys residues of dimer
  SITE 8: note = CROSSLINK - Diethylene glycol linkage between Lys residues of dimer
DTHFPCIKF

SEQ ID NO: 359 - protein, 9 aa, synthetic construct
  REGION 1..9: note = Hepcidin dimerized peptide
  DISULFID 6: note = disulphide linkage between Cys residues of dimer
DTHFPCIRK

SEQ ID NO: 360 - protein, 9 aa, synthetic construct
  REGION 1..9: note = Hepcidin dimerized peptide
  DISULFID 6: note = disulphide linkage between Cys residues of dimer
  VARIANT 9: note = diethylene glycol linkage between Lys residues of dimer
DTHFPCIRK

SEQ ID NO: 361 - protein, 10 aa, synthetic construct
  REGION 1..10: note = Hepcidin dimerized peptide
  DISULFID 6: note = disulphide linkage between Cys residues of dimer
DTHFPCIKFK

SEQ ID NO: 362 - protein, 10 aa, synthetic construct
  REGION 1..10: note = Hepcidin dimerized peptide
  DISULFID 6: note = disulphide linkage between Cys residues of dimer
  SITE 10: note = CROSSLINK - diethylene glycol linkage between Lys residues of dimer
DTHFPCIKFK

SEQ ID NO: 363 - protein, 9 aa, synthetic construct
  REGION 1..9: note = Hepcidin dimerized peptide
DTHFPCIKF

SEQ ID NO: 364 - protein, 9 aa, synthetic construct
  REGION 1..9: note = Hepcidin dimerized peptide
DTHFPCIKF

SEQ ID NO: 365 - protein, 10 aa, synthetic construct
  REGION 1..10: note = Hepcidin dimerized peptide
DTHFPCIKFK

SEQ ID NO: 366 - protein, 9 aa, synthetic construct
  REGION 1..9: note = Hepcidin dimerized peptide
DTHFPCIKF

SEQ ID NO: 367 - protein, 19 aa, synthetic construct
  REGION 1..19: note = Hepcidin dimerized peptide
  SITE 19: note = CROSSLINK - diethylene glycol linkage between Lys residues of dimer
DTHFPCIIFGPRSRGWVCK

SEQ ID NO: 368 - protein, 18 aa, synthetic construct
  REGION 1..18: note = Hepcidin dimerized peptide
  SITE 14: note = CROSSLINK - diethylene glycol linkage between Lys residues of dimer
DTHFPICIFGPRSKGWVC

SEQ ID NO: 369 - protein, 17 aa, synthetic construct
  REGION 1..17: note = Hepciding analogue peptide
DTHFPCIKFEPRSKECK

SEQ ID NO: 370 - protein, 17 aa, synthetic construct
  REGION 1..17: note = Hepcidin analogue peptide
DTHFPCIKFEPHSKECK

SEQ ID NO: 371 - protein, 17 aa, synthetic construct
  REGION 1..17: note = Hepcidin analogue pepide
DTHFPCIKKEPHSKECK

SEQ ID NO: 372 - protein, 17 aa, synthetic construct
  REGION 1..17: note = Hepcidin analogue peptide
DTHFPCIKFKPHSKECK

SEQ ID NO: 373 - protein, 15 aa, synthetic construct
  REGION 1..15: note = Hepcidin analogue peptide
DTHFPCIKFEPRECK

SEQ ID NO: 374 - protein, 15 aa, synthetic construct
  REGION 1..15: note = Hepcidin analogue peptide
DTHFPCIKFEPHECK

SEQ ID NO: 375 - protein, 14 aa, synthetic construct
  REGION 1..14: note = Hepcidin analogue peptide
DTHFPCIKFEPRCK

(19) United States
(12) Patent Application Publication
(10) Pub. No.: US 2025/0340608 A1
(43) Pub. Date: Nov. 6, 2025

(54) HEPCIDIN ANALOGUES AND USES THEREOF

(71) Applicant: Protagonist Therapeutics, Inc., Newark, CA (US)

(72) Inventors: Mark Leslie Smythe, Bardon (AU); Gregory Thomas Bourne, Moggill (AU); Simone Vink, Taringa (AU); Brian Troy Frederick, Ben Lomond, CA (US); Praveen Madala, Brisbane (AU); Anne Pernille Tofteng Shelton, Valby (DK); Jacob Ulrik Fog, Bagsværd (DK)

(73) Assignee: Protagonist Therapeutics, Inc., Newark, CA (US)

(21) Appl. No.: 19/069,557

(22) Filed: Mar. 4, 2025

Related U.S. Application Data
(63) Continuation of application No. 18/477,490, filed on Sep. 28, 2023, now Pat. No. 12,269,856.
Application No. 18/477,490 is a continuation of application No. 17/099,308, filed on Nov. 16, 2020, now Pat. No. 11,807,674.
Application No. 17/099,308 is a continuation of application No. 16/839,368, filed on Apr. 3, 2020, now abandoned.
Application No. 16/839,368 is a continuation of application No. 16/553,486, filed on Aug. 28, 2019, now abandoned.
Application No. 16/553,486 is a continuation of application No. 16/289,451, filed on Feb. 28, 2019, now Pat. No. 10,501,515.
Application No. 16/289,451 is a continuation of application No. 16/037,982, filed on Jul. 17, 2018, now Pat. No. 10,442,846.
Application No. 16/037,982 is a continuation of application No. 15/828,214, filed on Nov. 30, 2017, now Pat. No. 10,030,061.
Application No. 15/828,214 is a continuation of application No. 15/720,333, filed on Sep. 29, 2017, now abandoned.
Application No. 15/720,333 is a continuation of application No. 14/775,469, filed on Sep. 11, 2015, now Pat. No. 9,822,157.
(60) Provisional application No. 61/800,048, filed on Mar. 15, 2013.
(60) Provisional application No. 61/800,284, filed on Mar. 15, 2013.

(51) Int. Cl. C07K 14/575; A61K 38/00

(52) U.S. Cl. CPC C07K 14/575; A61K 38/00

(57) ABSTRACT

The present invention relates, inter alia, to certain hepcidin peptide analogues, including peptides and dimers thereof, and to the use of the peptides and peptide dimers in the treatment and/or prevention of a variety of diseases, conditions or disorders, including treatment and/or prevention of iron overload diseases, which include hereditary hemochromatosis and iron-loading anemias, and other conditions and disorders described herein.

### CROSS REFERENCE TO RELATED APPLICATIONS

This application is a Continuation of U.S. application Ser. No. 17/099,308, filed Nov. 16, 2020; which is a Continuation of U.S. application Ser. No. 16/839,368, filed Apr. 3, 2020, now abandoned; which is a Continuation of U.S. application Ser. No. 16/553,486, filed Aug. 28, 2019, now abandoned; which is a Continuation of U.S. application Ser. No. 16/289,451, filed Feb. 28, 2019, now U.S. Pat. No. 10,501,515, issued Dec. 10, 2019; which is a Continuation of U.S. application Ser. No. 16/037,982, filed Jul. 17, 2018, now U.S. Pat. No. 10,442,846, issued Oct. 15, 2019; which is a Continuation of U.S. application Ser. No. 15/828,214, filed Nov. 30, 2017, now U.S. Pat. No. 10,030,061, issued Jul. 24, 2018; which is a Continuation of U.S. application Ser. No. 15/720,333, filed Sep. 29, 2017, now abandoned; which is a Continuation of U.S. application Ser. No. 14/775,469, filed Sep. 11, 2015, now U.S. Pat. No. 9,822,157, issued Nov. 21, 2017, which is a U.S. National Phase Application of International Patent Application No. PCT/US2014/030352, filed Mar. 17, 2014, now expired; which claims the benefit under 35 U.S.C. § 119 (e) of U.S. Provisional Application No. 61/800,048, filed on Mar. 15, 2013, and U.S. Provisional Application No. 61/800,284, filed on Mar. 15, 2013, each of which is incorporated by reference herein in its entirety.

### STATEMENT REGARDING SEQUENCE LISTING

The Sequence Listing XML associated with this application is provided in XML file format and is hereby incorporated by reference into the specification. The name of the XML file containing the Sequence Listing XML is PRTH_001_09US_ST26.xml. The XML file is 485,338 bytes, and created on Sep. 28, 2023, and is being submitted electronically via USPTO Patent Center.

### FIELD OF THE INVENTION

The present invention relates, inter alia, to certain hepcidin peptide analogues, including peptides and dimers thereof, as well as compositions comprising the peptides and peptide dimers, and to the use of the peptides and peptide dimers in the treatment and/or prevention of a variety of diseases, conditions or disorders, including treatment and/or prevention of iron overload diseases including hereditary hemochromatosis, iron-loading anemias, and other conditions and disorders described herein.

### BACKGROUND

Hepcidin (also referred to as LEAP-1), a peptide hormone produced by the liver, is a regulator of iron homeostasis in humans and other mammals. Hepcidin acts by binding to its receptor, the iron export channel ferroportin, causing its internalization and degradation. Human hepcidin is a 25-amino acid peptide (Hep25). See Krause et al. (2000) FEBS Lett 480:147-150, and Park et al. (2001) J Biol Chem 276:7806-7810. The structure of the bioactive 25-amino acid form of hepcidin is a simple hairpin with 8 cysteines that form 4 disulfide bonds as described by Jordan et al. J Biol Chem 284:24155-67. The N terminal region is required for iron-regulatory function, and deletion of 5 N-terminal amino acid residues results in a loss of iron-regulatory function. See Nemeth et al. (2006) Blood 107:328-33.

Abnormal hepcidin activity is associated with iron overload diseases, including hereditary hemochromatosis (HH) and iron-loading anemias. Hereditary hemochromatosis is a genetic iron overload disease that is mainly caused by hepcidin deficiency, or in some cases by hepcidin resistance. This allows excessive absorption of iron from the diet and development of iron overload. Clinical manifestations of HH may include liver disease (e.g., hepatic cirrhosis and hepatocellular carcinoma), diabetes, and heart failure. Currently, the only treatment for HH is regular phlebotomy, which is very burdensome for the patients. Iron-loading anemias are hereditary anemias with ineffective erythropoiesis such as β-thalassemia, which are accompanied by severe iron overload. Complications from iron overload are the main cause of morbidity and mortality for these patients. Hepcidin deficiency is the main cause of iron overload in non-transfused patients, and contributes to iron overload in transfused patients. The current treatment for iron overload in these patients is iron chelation which is very burdensome, sometimes ineffective, and accompanied by frequent side effects.

Hepcidin has a number of limitations which restrict its use as a drug, including a difficult synthesis process due in part to aggregation and precipitation of the protein during folding, which in turn leads to high cost of goods. What are needed in the art are compounds having hepcidin activity and also possessing other beneficial physical properties such as improved solubility, stability, and/or potency, so that hepcidin-like biologies might be produced affordably, and used to treat hepcidin-related diseases and disorders such as, e.g., those described herein.

The present invention addresses such needs, providing novel peptide analogues, and dimers thereof, having hepcidin activity and also having other beneficial properties making the peptides of the present invention suitable alternatives to hepcidin.

### BRIEF SUMMARY OF THE INVENTION

The present invention generally relates to peptides exhibiting hepcidin activity and methods of using the same.

In some embodiments, the invention provides peptides, which may be isolated and/or purified, comprising, consisting essentially of, or consisting of, the following structural formula I:

(I)
(SEQ ID NO: 12)
R¹-X-Y-R²

or a pharmaceutically acceptable salt or solvate thereof, wherein
R¹ is hydrogen, an C1-C6 alkyl, C6-C12 aryl, C6-C12 aryl C1-C6 alkyl, C1-C20 alkanoyl (e.g. methyl, acetyl, formyl, benzoyl or trifluoroacetyl, isovaleric acid, isobutyric acid, octanoic acid, lauric acid and hexadecanoic acid), γ-Glu-hexadecanoic acid) or pGlu, appended to the N-terminus, and including PEGylated versions (e.g. PEO3 to PEGI1), alone or as spacers of any of the foregoing;
R² is —NH₂ or —OH;
X is a peptide sequence having the formula (Ia)

(Ia)
(SEQ ID NO: 1)
X1-X2-X3-X4-X5-X6-X7-X8-X9-X10

wherein
X1 is Asp, Glu, Ala, Gly, Thr, Ida, pGlu, bhAsp, D-Asp, Tyr, Leu or absent;
X2 is Thr, Ala, Aib, D-Thr, Arg or absent;
X3 is His, Lys, Ala, or D-His;
X4 is Phe, Ala, Dpa, bhPhe, of D-Phe;
X5 is Pro, Glu, Ser, Gly, Arg, Lys, Val, Ala, D-Pro, bbPro, Sarc, Abu or absent;
X6 is IIe, Cys, Arg, Leu, Lys, His, Gla, D-lle, D-Arg, D-Cys, Val, Ser or Ala;
X7 is Cys, IIe, Ala, Leu, Val, Ser, Phe, Dapa, D-Ile or D-Cys;
X8 is IIe, Lys, Arg, Ala, Gln, Phe, Glu, Asp, Tyr, Ser, Leu, Val, D-IIe, D-Lys, D-Arg, or Dapa;
X9 is Phe, Ala, IIe, Tyr, Lys, Arg, bhPhe or D-Phe; and
X10 is Lys, Phe or absent;
Y is absent or Y is a peptide having the formula (IIa)

(IIa)
(SEQ ID NO: 5)
Y1-Y2-Y3-Y4-Y5-Y6-Y7-Y8-Y9-Y10-Y11-Y12-Y13-Y14-Y15

wherein
Y1 is Gly, Cys, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp, Ala, His, Tyr or absent;
Y5 is Lys, Met, Arg. Ala or absent;
Y6 is Gly, Ser, Lys, IIe, Arg, Ala, Pro, Val or absent;
Y7 is Trp, Lys, Gly, Ala IIe, Val or absent;
Y8 is Val, Thr, Gly, Cys, Met, Tyr, Ala, Glu, Lys, Asp, Arg or absent;
Y9 is Cys, Tyr or absent;
Y10 is Met, Lys, Arg, Tyr or absent;
Y11 is Arg, Met, Cys, Lys or absent;
Y12 is Arg Lys, Ala or absent;
Y13 is Arg, Cys, Lys, Val or absent;
Y14 is Arg, Lys, Pro, Cys, Thr or absent; and
Y15 is Thr, Arg or absent;
wherein if Y is absent from the peptide of formula (I), X7 is Ile; and
wherein said compound of formula (I) is optionally PEGylated on R¹, X, or Y.

In some embodiments, the compound of formula (I) comprises two or more cysteine residues, wherein at least two of said cysteine residues are linked via a disulfide bond.

In some embodiments, the invention provides peptides, which may be isolated and/or purified, comprising, consisting essentially of, or consisting of the following structural formula I′:

(I′)
(SEQ ID NO: 21)
R¹′-X′-Y′-R²′

or a pharmaceutically acceptable salt or solvate thereof,
wherein
R¹″ is hydrogen, an C₁-C₆ alkyl, C₆-C₁₂ aryl, C₆-C₁₂ aryl C₁-C₆ alkyl, C₁-C₂₀ alkanoyl (e.g. methyl, acetyl, formyl, benzoyl or trifluoroacetyl, isovaleric acid, isobutyric acid, octanoic acid, lauric acid and hexadecanoic acid), γ-Glu-hexadecanoic acid) or pGlu, appended to the N. terminus, and including PEGylated versions (e.g. PEG3 to PEGI1), alone or as spacers of any of the foregoing;
R²′ is —NH: or —OH;
   X′ is a peptide sequence having the formula Ia′

(Ia′)
(SEQ ID NO: 13)
X1-X2-X3-X4-X5-X6-X7-X8-X9-X10

wherein
X1 is Asp, Glu, Ala, Gly, Thr, Ida, pGlu, bhAsp, D-Asp, Tyr, Leu or absent;
X2 is Thr, Ala, Aib, D-Thr, Arg or absent;
X3 is His, Ala, D-His or Lys;
X4 is Phe, Ala, Dpa, bhPhe or D-Phe;
X5 is Pro, Glu, Ser, Gly, Arg, Lys, Val, Ala, D-Pro, bhPro, Sarc, Abu or absent;
X6 is IIe, Cys, Arg, Leu, Lys, His, Gla, D-IIe, D-Arg, D-Cys, Val, Ser or Ala;
X7 is Cys, IIe, Ala, Leu, Val, Ser, Phe, Dapa, D-Ile or D-Cys,
X8 is IIe, Lys, Arg, Ala, Gln, Phe, Glu, Asp, Tyr, Ser, Leu, Val, D-lle, D-Lys, D-Arg, or Dapa;
X9 is Phe, Ala, De, Tyr, Lys, Arg, bhPhe or D-Phe; and
X10 is Lys. Phe or absent;
and provided that if Y′ is absent, X7 is Ile;
   Y′ is a peptide having the formula IIa′

(IIa′)
(SEQ ID NO: 16)
Y1-Y2-Y3-Y4-Y5-Y6-Y7-Y8-Y9-Y10-Y11-Y12-Y13-Y14-Y15

wherein
Y1 is Gly, Cys, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp, Ala, His, Tyr or absent;
Y5 is Lys, Met, Arg, Ala or absent;
Y6 is Gly, Ser, Lys, IIe, Ala, Pro. Val or absent;
Y7 is Trp, Lys, Gly, Ala, IIe, Val or absent;
Y8 is Val, Thr, Gly, Cys, Met, Tyr, Ala, Glu, Lys, Asp, Arg or absent;
Y9 is Cys, Tyr or absent;
Y10 is Met. Lys, Arg, Tyr or absent;
Y11 is Arg Met, Cys, Lys or absent;
Y12 is Arg. Lys, Ala or absent.
Y13 is Arg. Cys, Lys, Val or absent;
Y14 is Arg, Lys, Pro, Cys, Thr or absent; and
Y15 is Thr, Arg or absent;
wherein said compound of formula I′ is optionally PEGylated on R¹′, X′, or Y′; and
wherein when said compound of formula I′ comprises two or more cysteine residues, at least two of said cysteine residues being linked via a disulfide bond.

In some embodiments, the compound of formula I′ comprises an R¹′ moiety that is hydrogen, isovaleric acid, isobutyric acid, or acetyl.

In some embodiments, the compound of formula I′ comprises an X′ peptide of formula Ia′ as described herein, wherein
X1 is Asp, Ala, Ida, pGlu, bhAsp, Leu, D-Asp or absent;
X2 is Thr, Ala, or D-Thr;
X3 is His, Lys, D-His or Lys;
X4 is Phe, Ala, Dpa or D-Phe;
X5 is Pro, Gly, Arg, Lys, Ala, D-Pro or bhPro;
X6 is IIe, Cys, Arg, Lys, D-Ile or D-Cys;
X7 is Cys, De, Leu, Val, Phe, D-Ile or D-Cys;
X8 is IIe, Arg, Phe, Gln, Lys, Glu, Val, Leu or D-Ile;
X9 is Phe or bhPhe; and
X10 is Lys, Phe or absent.

In some embodiments, the compound of formula I′ comprises an X′ peptide of formula Ib′:

(Ib′)
X1-Thr-His-X4-X5-X6-X7-X8-Phe-X10

wherein
X1 is Asp, Ida, pGlu, bhAsp or absent,
X4 is Phe or Dpa;
X5 is Pro or bhPro;
X6 is IIe, Cys or Arg;
X7 is Cys, IIe, Leu or Val;
X8 is Ile Lys, Glu, Phe, Gin or Arg; and
X10 is Lys or absent.

In some embodiments, the compound of formula I′ comprises an X′ peptide of formula Ic′:

(Ic′)
X1-Thr-His-X4-X5-Cys-Ile-X8-Phe-X10

wherein
X1 is Asp, Ida, pGlu, bhAsp or absent;
X4 is Phe or Dpa;
X5 is Pro or bhPro;
X8 is Ile Lys, Glu, Phe, Gin or Arg; and
X10 is Lys or absent.

In some embodiments, the compound of formula I′ comprises a Y′ peptide of formula

(IIb′)
Y1-Y2-Y3-Y4-Y5-Y6-Y7-Y8-Cys-Y10

wherein
Y1 is Gly, Ala, Lys, Pro or D-Pro;
Y2 is Pro, Ala or Gly;
Y3 is Arg, Ala, Lys or Trp;
Y4 is Ser, Gly or Ala;
Y5 is Lys, Met, Arg or Ala;
Y6 is Gly, Arg or Ala;
Y7 is Trp or Ala;
Y8 is Val, Thr, Ala or Glu, and
Y 10 is Met Lys or absent.

In some embodiments, the compound of formula I′ comprises a Y′ peptide of formula IIc′.

(IIc′)
Y1-Y2-Y3-Ser-Lys-Gly-Trp-Y8-Cys-Y10

wherein
Y1 is Gly, Pro or D-Pro,
Y2 is Pro or Gly;
Y3 is Arg or Lys,
Y8 is Val or Thr; and
Y10 is Met, Lys or absent.

In some embodiments, the compound of formula I′ comprises a Y′ peptide of formula IId′:

(IId′)
Cys-Y3-Y4-Arg-Y6-Y7-Y8-Cys-Y10-Y11-Y12-Y13-Y14-Y15

wherein
Y1 is Val or Ala or absent;
Y3 is Gly, Pro or absent;
Y4 is His, Trp or Tyr; Y6 is Ser, Gly or Pro;
Y7 is IIe, Gly or Lys;
Y8 is Gly, Met or absent;
Y10 is Tyr or Cys;
Y11 is Arg, Lys, Met or Ala;
Y12 is Arg or Ala;
Y13 is Cys or Val or absent;
Y14 is Cys, Lys, Pro, Arg. Thr or absent; and
Y15 is Arg, Thr or absent.

In some embodiments, the compound of formula I′ comprises a Y′ peptide of formula IIe′:

(IIe′)
Val-Cys-Y3-His-Arg-Y6-Y7-Y8-Cys-Tyr-Arg-Y12-Y13-
Y14-Y15

wherein
Y3 is Gly or absent;
Y6 is Ser or Pro;
Y7 is Ile or Lys;
Y8 is Gly or absent;
Y12 is Arg or Ala;
Y13 is Cys or Val or absent;
Y14 is Cys, Arg, Thr or absent; and
Y15 is Arg or absent

In some embodiments, the invention provides peptides, which may be isolated and/or purified, comprising, consisting essentially of, or consisting of the following structural formula I″:

(I″)
(SEQ ID NO: 27)
R^{1″}-X″-Y″-R^{2″}

or a pharmaceutically acceptable salt or solvate thereof, wherein
R¹″ is hydrogen, an C₁-C₆ alkyl, C₆-C₁₂ aryl, C₆-C₁₂ aryl C₁-C₆ alkyl, C₁-C₂₀ alkanoyl (e.g. methyl, acetyl, formyl, benzoyl or trifluoroacetyl, isovaleric acid, isobutyric acid, octanoic acid, lauric acid and hexadecanoic acid), γ-Glu-hexadecanoic acid) or pGlu, appended to the N-terminus, and including PEGylated versions (e.g. PEG3 to PEGI1), alone or as spacers of any of the foregoing;
R²″ is —NH₂ or —OH;
X″ is a peptide sequence having the formula Ia″

(Ia″)
(SEQ ID NO: 22)
X1-X2-X3-X4-X5-X6-X7-X8-X9-X10

wherein
X1 is Asp, Glu, Ala, Gly, Thr, Ida, pGlu, bhAsp, D-Asp. Tyr, Leu or absent;
X2 is Thr, Ala, Aib, D-Thr, Arg or absent;
X3 is His, Ala, D-His or Lys;
X4 is Phe, Ala, Dpa, bhPhe or D-Phe;
X5 is Pro, Glu, Ser, Gly, Arg, Lys, Val, Ala, D-Pro, bhPro, Sarc, Abu or absent;
X6 is IIe, Cys, Arg, Leu, Lys, His, Gla, D-IIe, D-Arg, D-Cys, Val, Ser or Ala;
X7 is Cys, IIe, Ala, Leu, Val, Ser, Phe, Dapa, D-Ile or D-Cys;
X8 is IIe, Lys, Arg, Ala, Gln, Phe, Glu, Asp, Tyr, Ser, Lea, Val, D-IIe, D-Lys, D-Arg, or Dapa;
X9 is Phe, Ala, IIe, Tyr, Lys, Arg, bhPhe or D-Phe; and
X10 is Lys, Phe or absent;
and provided that if Y″ is absent, X7 is Ile.

In some embodiments, the compound of formula I′″ is PEGylated on R″

In some embodiments, the compound of formula I″ comprises two or more cysteine residues, at least two of said cysteine residues being linked via a disulfide bond.

In some embodiments, the compound of formula I″ comprises an R¹″ that is hydrogen, isovaleric acid, iso-butyric acid or acetyl.

In some embodiments, the compound of formula I″ comprises an X″ peptide according to formula Ia″, disclosed herein,
wherein
X1 is Asp, Ala, Ida, pGlu, bhAsp, Leu, D-Asp or absent;
X2 is Thr, Ala, or D-Thr;
X3 is His, Lys, D-His or Lys;
X4 is Phe, Ala, or Dpa;
X5 is Pro, Gly, Arg, Lys, Ala, D-Pro or bhPro;
X6 is IIe, Cys, Arg, Lys, D-Ile or D-Cys;
X7 is Cys, IIe, Leu, Val, Phe, D-Ile or D-Cys;
X8 is IIe, Arg, Phe, Gln, Lys, Glu, Val, Leu or D-Ile;
X9 is Phe or bhPhe; and
X10 is Lys or absent.

In some embodiments, the compound of formula I′ comprises an X″ peptide of formula

(Ib″)
X1-Thr-His-X4-X5-X6-X7-X8-Phe-X10

wherein
X1 is Asp, Ida, pGlu, bhAsp or absent;
X4 is Phe or Dpa;
X5 is Pro or bhPro;
X6 is IIe, Cys or Arg;
X7 is Cys, IIe, Leu or Val;
X8 is IIe, Lys, Glu, Phe, Gin or Arg; and
X10 is Lys Phe or absent.

In some embodiments, the compound of formula I″ comprises an X″ peptide of formula Ic″:

(Ic″)
X1-Thr-His-X4-X5-Cys-Ile-X8-Phe-X10

wherein
X1 is Asp, Ida, pGlu, bhAsp or absent,
X4 is Phe or Dpa;
X5 is Pro or bhPro;
X8 is IIe, Lys, Glu, Phe, Gin or Arg; and
X10 is Lys or absent.

In some embodiments, the compound of formula I″ comprises a Y″ peptide of formula IIa″:

(IIa″)
(SEQ ID NO: 25)
Y1-Y2-Y3-Y4-Y5-Y6-Y7-Y8-Cys-Y10

wherein
Y1 is Gly, Ala, Lys, Pro or D-Pro;
Y2 is Pro, Ala or Gly;
Y3 is Arg, Ala, Lys or Trp;
Y4 is Ser, Gly or Ala;
Y5 is Lys, Met, Arg or Ala;
Y6 is Gly, Arg or Ala;
Y7 is Trp, Ala or absent;
Y8 is Val, Thr, Lys, Ala, Glu or absent; and
Y10 is Met, Lys or absent.

In some embodiments, the compound of formula I″ comprises a Y″ peptide of formula IIb″:

(IIb″)
Y1-Y2-Y3-Ser-Lys-Gly-Trp-Y8-Cys-Y10

wherein
Y1 is Gly, Pro or D-Pro;
Y2 is Pro, Gly;
Y3 is Arg, Lys;
Y8 is Val or Thr, and
Y10 is Met, Lys or absent.

In related embodiments, the present invention includes dimers, e.g., homodimers, of any of the peptides of the present invention.

In some embodiments, the peptides or dimers of the present invention exhibit hepcidin activity. In some embodiments, the peptides or dimers bind ferroportin, e.g., human ferroportin.

In some embodiments, the present invention provides methods of binding a ferroportin or inducing ferroportin internalization and degradation which comprise contacting the ferroportin with at least one peptide, dimer or composition as disclosed herein.

In some embodiments, the present invention provides compositions and medicaments comprising at least one peptide or dimer as disclosed herein.

In some embodiments, the present invention provides a method of manufacturing medicaments comprising at least one peptide or dimer as disclosed herein for the treatment of diseases of iron metabolism, such as iron overload diseases.

Also provided are methods of treating a disease of iron metabolism in a subject, such as a mammalian subject, e.g., a human subject, comprising administering at least one peptide, dimer or composition as disclosed herein to the subject. In some embodiments, the peptide or dimer is administered in a therapeutically effective amount. In some embodiments, the disease of iron metabolism is an iron overload disease

In some embodiments, the present invention provides a method of manufacturing a peptide or peptide dimer of the present invention synthetically. In some embodiments, the present invention provides a method of manufacturing a peptide or peptide dimer of the present invention recombinantly.

In some embodiments, the present invention provides a pharmaceutical composition comprising a peptide analogue (e.g., a peptide or dimer of the present invention), or pharmaceutically acceptable salt or solvate thereof, as described herein, in combination with one or more peptide analogue (e.g., a peptide or dimer of the present invention) or pharmaceutically acceptable salt or solvate thereof, as described herein together with a pharmaceutically acceptable carrier, excipient or vehicle.

In some embodiments, the invention provides a process for manufacturing a compound or a pharmaceutical composition as disclosed herein.

In some embodiments, the invention provides a device comprising at least one peptide analogue (e.g., a peptide or dimer of the present invention), or pharmaceutically acceptable salt or solvate thereof for delivery of the peptide analogue to a subject.

In some embodiments, the present invention provides kits comprising at least one peptide, dimer, or composition as disclosed herein packaged together with a reagent, a device, instructional material, or a combination thereof.

In some embodiments, the present invention provides complexes which comprise at least one peptide or dimer as disclosed herein bound to a ferroportin, e.g., a human ferroportin, or an antibody, such as an antibody which specifically binds a peptide as disclosed herein, Hep25, or a combination thereof.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### BRIEF DESCRIPTION OF THE DRAWINGS

FIG. 1 shows the results of an in vitro activity assay measuring the induction of degradation of the human ferroportin protein. Presented are dose response curves for Compound No. 1 as compared to Hepcidin and the Mini-Hepcidin control.

FIG. 2 shows time dependent changes in serum iron following animal exposure to vehicle, Compound No. 2 and reference compound RI Mini-Hepcidin. The responses are normalized to the initial (t=0) levels.

FIG. 3 shows relative decrease of serum iron relative to vehicle control measured with Compound No. 2 as well as the reference compound RI-Mini-Hepcidin at timepoints 0, 30, 60. 120, 240 and 360 minutes. 100% represents the measured average level of serum iron in the vehicle treated animals.

FIG. 4 shows the in vivo serum iron reducing abilities of selected peptides of the present invention and Hepcidin.

FIG. 5 shows a dose response of the in vivo serum iron reducing abilities of selected peptides of the present invention and Hepcidin.

FIG. 6 shows the PK/PD effects for the in vivo serum iron reducing abilities of selected peptides of the present invention and Hepcidin. For Hepcidin and the 300 nmol/kg treatment with compound #181, only one timepoint was taken at t=120 min. The Hepcidin response is not clearly visible on this graph, as it overlapped with the Cmpd #181 1000 nmol/kg plot at the t−120 min point. The single data point for compound #181 300 nmol/kg is located directly above the Hepcidin point.

FIG. 7 shows selected examples of linkers that were used to dimerize the peptides.

### DETAILED DESCRIPTION OF THE INVENTION

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

All publications, patents and published patent applications referred to in this application are specifically incorporated by reference herein. In case of conflict, the present specification, including its specific definitions, will control.

Each embodiment of the invention described herein may be taken alone or in combination with one or more other embodiments of the invention.

### Definitions

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats).

The term formula (I), is used herein interchangeably with the term formula I (i.e., without the parentheses). The term formula (I′), is used herein interchangeably with the term formula I′ (i.e., without the parentheses). The term formula (I″), is used herein interchangeably with the term formula I″ (i.e., without the parentheses).

The recitations “sequence identity”, “percent identity”, “percent homology”, or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, IIe, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GOG software package, using an NWSgapdna.CMP matrix and a gap weight of 40, 50, 60, 70, or 80 and a length weight of 1, 2, 3, 4, 5, or 6. Another exemplary set of parameters includes a Blossunt 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4:11-17) which has been incorporated into the ALIGN program (version 2.0), using a PAM120 weight residue table, a gap length penalty of 12 and a gap penalty of 4.

The peptide sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215:403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (Nucleic Acids Res. 25:3389-3402, 1997). When utilizing BLAST and Gapped BLAST programs, the default parameters of the respective programs (e.g., XBLAST and NBLAST) can be used.

As used herein, the term “pharmaceutically acceptable salt” is intended to indicate a salt which is not harmful to a patient or subject to which the salt in question is administered. It may suitably be a salt chosen, e.g., among acid addition salts and basic salts. Examples of acid addition salts include chloride salts, citrate salts and acetate salts. Examples of basic salts include salts where the cation is selected among alkali metal cations, such as sodium or potassium ions, alkaline earth metal cations, such as calcium or magnesium ions, as well as substituted ammonium ions, such as ions of the type N(R1)(R2XR3)(R4)+, where R1, R2, R3 and R4 independently will typically designate hydrogen, optionally substituted C₁-6-alkyl or optionally substituted C2-6-alkenyl. Examples of relevant C1-6-alkyl groups include methyl, ethyl, 1-propyl and 2-propyl groups. Examples of C2-6-alkenyl groups of possible relevance include ethenyl, 1-propenyl and 2-propenyl. Other examples of pharmaceutically acceptable salts are described in “Remington's Pharmaceutical Sciences”, 17th edition, Alfonso R. Gennaro (Ed.), Mark Publishing Company, Easton, PA, USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66:2 (1977). Also, for a review on suitable salts, see Handbook of Pharmaceutical Salts: Properties, Selection, and Use by Stahl and Wermuth (Wiley-VCH, 2002). Other suitable base salts are formed from bases which form non-toxic salts. Representative examples include the aluminum, arginine, benzathine, calcium, choline, diethylamine, diolamine, glycine, lysine, magnesium, meglumine, olamine, potassium, sodium, tromethamine, and zinc salts. Hemisalts of acids and bases may also be formed, e.g., hemisulphate and hemicalcium salts. Compositions to be used in the invention suitable for parenteral administration may comprise sterile aqueous solutions and/or suspensions of the pharmaceutically active ingredients preferably made isotonic with the blood of the recipient, generally using sodium chloride, glycerin, glucose, mannitol, sorbitol, and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl)benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4˜ chlorobenzenesulfonic acid, 2-naphthalenesulfonic acid, 4-toluenesulfonic acid, camphorsulfonic acid, etc.), 4-methylbicyclo(2.2.2)-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

The term “solvate” in the context of the present invention refers to a complex of defined stoichiometry formed between a solute (in casu, a peptide analogue or pharmaceutically acceptable salt thereof according to the invention) and a solvent The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

The term “agonist” as employed in the context of the invention refers to a substance (ligand) that causes internalization of the ferroportin protein.

As used herein, a “disease of iron metabolism” includes diseases where aberrant iron metabolism directly causes the disease, or where iron blood levels are dysregulated causing disease, or where iron dysregulation is a consequence of another disease, or where diseases can be treated by modulating iron levels, and the like. More specifically, a disease of iron metabolism according to this disclosure includes iron overload diseases, iron deficiency disorders, disorders of iron biodistribution, other disorders of iron metabolism and other disorders potentially related to iron metabolism, etc. Diseases of iron metabolism include hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, sideroblastic anemia, porphyria, porphyria cutanea tarda, African iron overload, hyperferritinemia, ceruloplasmin deficiency, atransferrinemia, congenital dyserythropoietic anemia, anemia of chronic disease, anemia of inflammation, anemia of infection, hypochromic microcytic anemia, iron-deficiency anemia, iron-refractory iron deficiency anemia, anemia of chronic kidney disease, erythropoietin resistance, iron deficiency of obesity, other anemias, benign or malignant tumors that overproduce hepcidin or induce its overproduction, conditions with hepcidin excess, Friedreich ataxia, gracile syndrome, Hallervorden-Spatz disease, Wilson's disease, pulmonary hemosiderosis, hepatocellular carcinoma, cancer, hepatitis, cirrhosis of liver, pica, chronic renal failure, insulin resistance, diabetes, atherosclerosis, neurodegenerative disorders, multiple sclerosis, Parkinson's disease, Huntington's disease, and Alzheimer's disease.

In some embodiments, the disease and disorders are related to iron overload diseases such as iron hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia.

In some embodiments, the peptides of the invention are used to treat diseases and disorders that are not typically identified as being iron related. For example, hepcidin is highly expressed in the murine pancreas suggesting that diabetes (Type I or Type II), insulin resistance, glucose intolerance and other disorders may be ameliorated by treating underlying iron metabolism disorders. See Ilyin, G. et al. (2003) FEBS Lett. 542 22-26, which is herein incorporated by reference. As such, peptides of the invention may be used to treat these diseases and conditions. Those skilled in the art are readily able to determine whether a given disease can be treated with a peptide according to the present invention using methods known in the art, including the assays of WO 2004092405, which is herein incorporated by reference, and assays which monitor hepcidin, hemojuvelin, or iron levels and expression, which are known in the art such as those described in U.S. Pat. No. 7,534,764, which is herein incorporated by reference.

In certain embodiments of the present invention, the diseases of iron metabolism are iron overload diseases, which include hereditary hemochromatosis, iron-loading anemias, alcoholic liver diseases and chronic hepatitis C.

As used herein, the terms “protein”, “polypeptide” and “peptide” are used interchangeably to refer to two or more amino acids linked together. Except where indicated otherwise, e.g., for the abbreviations for the uncommon or unnatural amino acids set forth herein, the three-letter and one-letter abbreviations, as used in the art, are used herein to represent amino acid residues. Except when preceded with “D-”, the amino acid is an L-amino acid. Groups or strings of amino acid abbreviations are used to represent peptides. Except when specifically indicated, peptides are indicated with the N-terminus on the left and the sequence is written from the N-terminus to the C-terminus.

The term “peptide analogue” in the context of the present invention refers to a molecule in which a first peptide moiety is attached (i.e. coupled or linked), either directly or via a linking (i.e. bridging or spacing) chemical moiety, to a second peptide moiety, by means of covalent chemical bonding. In certain embodiments, a peptide analogue is a peptide described herein comprising an X peptide sequence and a Y peptide sequence. In certain embodiments, a peptide analogue is a peptide described herein comprising an X′ peptide sequence and a Y′ peptide sequence. In certain embodiments, a peptide analogue is a peptide described herein comprising an X″ peptide sequence and a Y″ peptide sequence. In certain embodiments, a peptide analogue is a peptide described herein comprising an X peptide sequence and/or a Y peptide sequence conjugated to an additional chemical moiety. In certain embodiments, a peptide analogue is a peptide described herein comprising an X′ peptide sequence and/or a Y′ peptide sequence conjugated to an additional chemical moiety. In certain embodiments, a peptide analogue is a peptide described herein comprising an X″ peptide sequence and/or a Y″ peptide sequence conjugated to an additional chemical moiety. The peptides of the present invention described herein are peptide analogues. Peptide analogues also include any of the peptide dimers described herein.

Peptides and peptide dimers of the present invention may also be referred to herein as compounds or peptide analogues.

The term “conservative substitution” as used herein denotes that one or more amino acids are replaced by another, biologically similar residue. Examples include substitution of amino acid residues with similar characteristics, e.g., small amino acids, acidic amino acids, polar amino acids, basic amino acids, hydrophobic amino acids and aromatic amino acids. See, for example, the table below. In some embodiments of the invention, one or more Met residues are substituted with norleucine (Nle) which is a bioisostere for Met, but which, as opposed to Met, is not readily oxidized. Another example of a conservative substitution with a residue normally not found in endogenous, mammalian peptides and proteins is the conservative substitution of Arg or Lys with, for example, ornithine, canavanine, aminoethylcysteine or another basic amino acid. In some embodiments, one or more cysteines of a peptide analogue of the invention may be substituted with another residue, such as a serine. For further information concerning phenotypically silent substitutions in peptides and proteins, see, for example, Bowie et. al. Science 247, 1306-1310, 1990. In the scheme below, conservative substitutions of amino acids are grouped by physicochemical properties. I: neutral, hydrophilic, II: acids and amides, III: basic, IV: hydrophobic, V: aromatic, bulky amino acids.

| I | II | III | IV | V |
|---|---|---|---|---|
| A | N | H | M | F |
| S | D | R | L | Y |
| T | E | K | I | W |
| P | Q | | V | |
| G | | | C | |

In the scheme below, conservative substitutions of amino acids are grouped by physicochemical properties. VI: neutral or hydrophobic, VII: acidic, VIII: basic, IX: polar, X: aromatic.

| VI | VII | VIII | IX | X |
|---|---|---|---|---|
| A | E | H | M | F |
| L | D | R | S | Y |
| I | | K | T | W |
| P | | | C | |
| G | | | N | |
| V | | | Q | |

In certain embodiments, the present invention provides peptides which are useful in the study and treatment of diseases of iron metabolism.

Throughout the present specification, unless naturally occurring amino acids are referred to by their full name (e.g. alanine, arginine, etc.), they are designated by their conventional three-letter or single-letter abbreviations (e.g. Ala or A for alanine, Arg or R for arginine, etc.). In the case of less common or non-naturally occurring amino acids, unless they are referred to by their full name (e.g. sarcosine, ornithine, etc.), frequently employed three- or four-character codes are employed for residues thereof, including, Sar or Sarc (sarcosine, i.e. N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), β-Pro (pyrrolidine-3-carboxylic acid), and 8Ado (8-amino-3,6-dioxaoctanoic acid), Abu (4-amino butyric acid), bhPro (B-homoproline), bhPhe (β-homophenylalanine) and Dpa (β,β diphenylalanine), and Ida (Iminodiacetic acid).

As is clear to the skilled artisan, the peptide sequences disclosed herein are shown proceeding from left to right, with the left end of the sequence being the N-terminus of the peptide and the right end of the sequence being the C-terminus of the peptide. Among sequences disclosed herein are sequences incorporating a “Hy˜” moiety at the amino terminus (N-terminus) of the sequence, and either an “—OH” moiety or an “—NH₂” moiety at the carboxy terminus (C-terminus) of the sequence. In such cases, and unless otherwise indicated, a “Hy-” moiety at the N-terminus of the sequence in question indicates a hydrogen atom [e.g., R¹, R¹′, or R¹″=hydrogen (Hy-) in formula I, I′, or I″, respectively, corresponding to the presence of a free primary or secondary amino group at the N-terminus], while an “—OH” or an “—NH₁” moiety at the C-terminus of the sequence indicates a hydroxy group [e.g., R², R²′, or R²″: ═OH in formula I, I′, or I″, respectively, corresponding to the presence of a carboxy (COOH) group at the C-terminus] or an amino group [e.g., R², R²′, or R²″═NH₂ in formula I, I′, or I″, respectively, corresponding to the presence of an amido (CONH₂) group at the C-terminus], respectively. In each sequence of the invention, a C-terminal “—OH” moiety may be substituted for a C-terminal “—NH₂” moiety, and vice-versa. Furthermore, R¹, R¹′, or R¹″ can in all sequences be substituted with isovaleric acids or equivalent. In some embodiments, wherein a peptide of the present invention is conjugated to an acidic compound such as, e.g., isovaleric acid, isobutyric acid, valeric acid, and the like, the presence of such a conjugation is referenced in the acid form. So, for example, but not to be limited in any way, instead of indicating a conjugation of isovaleric acid to a peptide DTHFPCIKFCK (SEQ ID NO:215) by referencing isovaleroyl (e.g., isovaleroyl-DTHEPCIKFCK[SEQ ID NO:215]), in some embodiments, the present application references such a conjugation as isovaleric acid-DTHFPCIKFCK (SEQ ID NO:215). Unless otherwise indicated, reference is made to the L-isomeric forms of the amino acids in question. Where appropriate, the D-isomeric form of an amino acid is indicated in the conventional manner by the prefix “D” before the conventional three-letter code (e.g., DAsp or D-Asp; DPhe or D-Phe).

In some embodiments, the invention provides peptides, which may be isolated and/or purified, comprising, consisting essentially of, or consisting of, the following structural formula:

(I)
(SEQ ID NO: 12)
R¹-X-Y-R²

or a pharmaceutically acceptable salt or solvate thereof, wherein
R¹ is hydrogen, an C1-C6 alkyl, C6-C12 aryl, C6-C12 aryl C1-C6 alkyl, C1-C20 alkanoyl (e.g., methyl, acetyl, formyl, benzoyl or trifluoroacetyl, isovaleric acid, isobutyric acid, octanoic acid, lauric acid and hexadecanoic acid), γ-Glu-hexadecanoic acid) or pGlu, appended to the N-terminus, and including PEGylated versions (e.g., PEG3 to PEG11), alone or as spacers of any of the foregoing:
R² is —NH₂ or —OH;
X is a peptide sequence having the formula (Ia)

(Ia)
(SEQ ID NO: 1)
X1-X2-X3-X4-X5-X6-X7-X8-X9-X10

wherein
X1 is Asp, Glu, Ala, Gly, Thr, Ida, pGlu, bhAsp, D-Asp, Tyr, Leu or absent;
X2 is Thr, Ala, Aib, D-Thr, Arg or absent;
X3 is His, Lys, Ala, or D-His;
X4 is Phe, Ala, Dpa, bhPhe, or D-Phe;
X5 is Pro, Glu, Ser, Gly, Arg, Lys, Val, Ala, D-Pro, bhPro, Sarc, Abu or absent,
X6 is IIe, Cys, Arg, Leu, Lys, His, Gla, D-IIe, D-Arg, D-Cys, Val, Ser or Ala;
X7 is Cys, IIe, Ala, Leu, Val, Ser, Phe, Dapa, D-Ile or D-Cys;
X8 is IIe, Lys, Arg. Ala, Gln, Phe, Glu, Asp. Tyr, Ser, Leu, Val, D-lle, D-Lys, D-Arg, or Dapa;
X9 is Phe, Ala, IIe, Tyr, Lys, Arg, bhPhe or D-Phe; and
X10 is Lys. Phe or absent;
Y is absent or Y is a peptide having the formula (IIa)

(IIa)
(SEQ ID NO: 5)
Y1-Y2-Y3-Y4-Y5-Y6-Y7-Y8-Y9-Y10-Y11-Y12-Y13-Y14-Y15

wherein
Y1 is Gly, Cys, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp. Ala, His, Tyr or absent;
Y5 is Lys, Met, Arg, Ala or absent;
Y6 is Gly, Ser, Lys, IIe, Arg, Ala, Pro, Val or absent;
Y7 is Trp. Lys, Gly, Ala IIe, Val or absent;
Y8 is Val, Thr, Gly, Cys, Met, Tyr, Ala, Glu, Lys, Asp, Arg or absent;
Y9 is Cys, Tyr or absent;
Y10 is Met, Lys, Arg, Tyr or absent;
Y11 is Arg. Met. Cys, Lys or absent;
Y12 is Arg. Lys, Ala or absent;
Y13 is Arg. Cys, Lys, Val or absent;
Y14 is Arg, Lys, Pro, Cys, Thr or absent; and
Y15 is Thr, Arg or absent;
wherein if Y is absent from the peptide of formula (I), X7 is Ile; and
wherein said compound of formula (I) is optionally PEGylated on R¹, X, or Y.

In some embodiments, the compound or peptide of formula (I) comprises two or more cysteine residues, wherein at least two of said cysteine residues are linked via a disulfide bond.

In some embodiments, X is a peptide sequence according to formula (Ia), described herein,
wherein
X1 is Asp. Glu, Ala, Gly, Thr, Ida, pGlu, bhAsp, D-Asp, Tyr, Leu or absent;
X2 is Thr, Ala, Aib, D-Thr, Arg or absent;
X3 is His, Lys, Ala, or D-His;
X4 is Phe, Ala, Dpa, or bhPhe;
X5 is Pro, Glu, Ser, Gly, Arg, Lys, Val, Ala, D-Pro, bhPro, Sarc, Abu or absent;
X6 is IIe, Cys, Arg, Leu, Lys, His, Glu, D-IIe, D-Arg, D-Cys, Val, Ser or Ala;
X7 is Cys, IIe, Ala, Leu, Val, Ser, Phe, Dapa, D-Ile or D-Cys;
X8 is IIe, Lys, Arg, Ala, Gln, Phe, Glu, Asp, Tyr, Ser, Leu, Val, D-IIe, D-Lys, D-Arg, or Dapa;
X9 is Phe, Ala, IIe, Tyr, Lys, Arg, bhPhe or D-Phe; and
X10 is Lys, Phe or absent.

In some embodiments, X is a peptide sequence according to formula (Ia), described herein, wherein
X1 is Asp, Ala, Ida, pGlu, bhAsp, Leu, D-Asp or absent;
X2 is Thr, Ala, or D-Thr;
X3 is His, Lys, or D-His;
X4 is Phe, Ala, or Dpa;
X5 is Pro, Gly, Arg, Lys, Ala, D-Pro or bhPro;
X6 is IIe, Cys, Arg, Lys, D-Ile or D-Cys;
X7 is Cys, IIe, Leu, Val, Phe, D-Ile or D-Cys;
X8 is IIe, Arg, Phe, Gln, Lys, Glu, Val, Leu or D-Ile;
X9 is Phe or bhPhe; and
X10 is Lys Phe or absent

In some embodiments, X is a peptide sequence having the formula (Ib)

(Ib)
(SEQ ID NO: 2)
X1-Thr-His-X4-X5-X6-X7-X8-Phe-X10

wherein
X1 is Asp, Ida, pGlu, bhAsp or absent;
X4 is Phe or Dpa;
X5 is Pro or bhPro;
X6 is IIe, Cys or Arg,
X7 is Cys, IIe, Leu or Val,
X8 is IIe, Lys, Glu, Phe, Gln or Arg; and
X10 is Lys Phe or absent;

In some embodiments, X is a peptide sequence according to formula (Ib), as described herein, wherein
X1 is Asp, Glu, Ida, pGlu, bhAsp or absent;
X4 is Phe or Dpa;
X5 is Pro or bhPro;
X6 is IIe, Cys or Arg;
X7 is Cys, IIe, Leu or Val;
X8 is IIe, Lys, Glu, Phe, Gin or Arg; and
X10 is Lys or absent.

In some embodiments, X is a peptide sequence having the formula (Ic)

(Ic)
(SEQ ID NO: 3)
X1-Thr-His-X4-X5-Cys-Ile-X8-Phe-X10

wherein
X1 is Asp, Glu, Ida, pGlu, bhAsp or absent;
X4 is: Phe or Dpa;
X5 is Pro or bhPro;
X8 is Ile Lys, Glu, Phe, Gin or Arg; and
X10 is Lys or absent.

In some embodiments, X is a peptide sequence having the formula (Id)

(Id)
(SEQ ID NO: 4)
X1-Thr-His-Phe-X5-Cys-Ile-X8-Phe-X10

wherein
X1 is Asp, Glu, or Ida;
X4 is: Phe;
X5 is Pro or bhPro;
X8 is IIe, Lys or Phe; and
X10 is absent.

In some embodiments, Y is a peptide sequence having the formula IIb

(IIb)
(SEQ ID NO: 6)
Y1-Y2-Y3-Y4-Y5-Y6-Y7-Y8-Cys-Y10

wherein
Y1 is Gly, Ala, Lys, Pro or D-Pro;
Y2 is Pro, Ala or Gly;
Y3 is Arg, Ala, Lys or Trp;
Y4 is Ser, Gly or Ala,
Y5 is Lys, Met. Arg or Ala;
Y6 is Gly, Arg or Ala;
Y7 is Trp, Ala or absent;
Y8 is Val, Thr, Lys, Ala, Glu or absent; and
Y10 is Met, Lys or absent.

In some embodiments, Y is a peptide sequence according to formula (IIb), as described herein,
wherein
Y1 is Gly, Ala, Lys, Pro or D-Pro;
Y2 is Pro, Ala or Gly;
Y3 is Arg, Ala, Lys or Trp;
Y4 is Ser, Gly or Ala;
Y5 is Lys, Met, Arg or Ala;
Y6 is Gly, Arg or Ala;
Y7 is Trp or Ala;
Y8 is Val, Thr, Ala, or Glu; and
Y10 is Met, Lys or absent.

In some embodiments, Y is a peptide sequence having the formula (IIc)

(IIc)
(SEQ ID NO: 7)
Y1-Y2-Y3-Ser-Lys-Gly-Trp-Y8-Cys-Y10

wherein
Y1 is Gly, Pro or D-Pro,
Y2 is Pro or Gly;
Y3 is Arg or Lys,
Y8 is Val or Thr; and
Y10 is Met, Lys or absent.

In some embodiments, Y is a peptide sequence having the formula (IId)

(IId)
(SEQ ID NO: 8)
Y1-Cys-Y3-Y4-Arg-Y6-Y7-Y8-Cys-Y10-Y11-Y12-Y13-Y14-
Y15

wherein
Y1 is Val, Ala or absent;
Y3 is Gly, Pro or absent;
Y4 is His, Trp or Tyr,
Y6 is Ser, Gly or Pro;
Y7 is IIe, Gly or Lys;
Y8 is Gly, Met or absent;
Y10 is Tyr or Cys;
Y11 is Arg. Lys, Met or Ala;
Y12 is Arg or Ala;
Y13 is Cys or Val or absent;
Y14 is Cys, Lys, Pro, Arg, Thr or absent, and
Y15 is Arg, Thr or absent.

In some embodiments, Y is a peptide sequence having the formula (IIe)

(IIe)
(SEQ ID NO: 9)
Val-Cys-Y3-His-Arg-Y6-Y7-Y8-Cys-Tyr-Arg-Y12-Y13-
Y14-Y15

wherein
Y3 is Gly or absent;
Y6 is Ser or Pro;
Y7 is Ile or Lys,
Y8 is Gly or absent;
Y12 is Arg or Ala;
Y13 is Cys, Val or absent;
Y14 is Cys, Arg, Thr or absent; and
Y15 is Arg or absent.

In some embodiments, Y is a peptide sequence having the formula (IIf)

(IIf)
(SEQ ID NO: 10)
Y1-Pro-Y3-Ser-Y5-Y6-Y7-Y8-Cys-Y10

wherein
Y1 is Gly, Glu, Val, or Lys;
Y3 is Arg or Lys:
Y5 is Arg or Lys:
Y6 is Gly, Ser, Lys, Ile or Arg;
Y7 is Trp or absent;
Y8 is Val, Thr, Asp, Glu or absent; and
Y10 is Lys or absent.

In some embodiments, Y is a peptide sequence having the formula (IIg)

(IIg)
(SEQ ID NO: 11)
Y1-Pro-Y3-Ser-Y5-Y6-Y7-Y8-Cys-Y10

wherein
Y1 is Glu or Lys;
Y3 is Arg or Lys;
Y5 is Arg or Lys:
Y6 is Gly, Ser, Lys, Ile or Arg;
Y7 is Trp or absent;
Y8 is Val or absent; and
Y10 is Lys or absent.

In some embodiments, the peptide of formula (I) comprises at least three, at least four, at least five, at least six, at least seven, at least eight, at least nine, at least ten, at least eleven, at least twelve, at least thirteen, at least fourteen or at least fifteen Y residues in Y

In some embodiments, Y1 to Y3 are present and Y4 to Y15 are absent.

In some embodiments, Y1 to Y11 are present and Y12 to Y15 are absent.

In some embodiments, Y1 to Y10 are present and Y11 to Y15 are absent.

In some embodiments, Y8 and Y15 are absent.

In some embodiments, Y3 and Y15 are absent

In some embodiments, Y3, Y14 and Y15 are absent.

In some embodiment Y5 is absent.

In some embodiments Y1, YS, Y7, Y12, Y13, Y14 and Y15 are absent.

In some embodiments Y1, Y5, and Y7 are absent. In some embodiments, Y8 is absent. In some embodiments, Y3 is absent. In some embodiments Y1, Y5, Y7, and Y11-Y15 are absent. In some embodiments, Y8 and Y11-Y15 are absent. In some embodiments, Y3 and Y11-Y15 are absent.

In some embodiments, the present invention provides a compound of formula (I), wherein the compound comprises any one of the X/Y peptide sequence formula combinations presented in Table 1 below.

TABLE 1
Illustrative combinations of X and Y
peptides of a compound of Formula (I)
Formula I combinations

| | X Peptide | Y Peptide |
|---|---|---|
| Combination | Sequence | Sequence |
| Number | Formula | Formula |
| 1 | Ia | IIa |
| 2 | Ia | IIb |
| 3 | Ia | IIc |
| 4 | Ia | IId |
| 5 | Ia | IIe |
| 6 | Ia | IIf |
| 7 | Ia | IIg |
| 8 | Ib | IIa |
| 9 | Ib | IIb |
| 10 | Ib | IIc |
| 11 | Ib | IId |
| 12 | Ib | IIe |
| 13 | Ib | IIf |
| 14 | Ib | IIg |
| 15 | Ic | IIa |
| 16 | Ic | IIb |
| 17 | Ic | IIc |
| 18 | Ic | IId |
| 19 | Ic | IIe |
| 20 | Ic | IIf |
| 21 | Ic | IIg |
| 22 | Id | IIa |
| 23 | Id | IIb |
| 24 | Id | IIc |
| 25 | Id | IId |
| 26 | Id | IIe |
| 27 | Id | IIf |
| 28 | Id | IIg |

In some embodiments, the invention provides peptides, which may be isolated and/or purified, comprising, consisting essentially of, or consisting of, the following structural formula:

(I′)
(SEQ ID NO: 21)
R¹′-X′-Y′-R²′

or a pharmaceutically acceptable salt or solvate thereof, wherein R¹′ is hydrogen, an C₁-C₆ alkyl, C₆-C₁₂ aryl, C₆-C₁₂ aryl C₁-C₆ alkyl, C₁-C₂₀ alkanoyl (e.g., methyl, acetyl, formyl, benzoyl or trifluoroacetyl, isovaleric acid, isobutyric acid, octanoic acid, lauric acid and hexadecanoic acid), γ-Glu-hexadecanoic acid) or pGlu, appended to the N-terminus, and including PEGylated versions (e.g., PEG3 to PEG11), alone or as spacers of any of the foregoing;
R²′ is —NH: or —OH;
X′ is a peptide sequence having the formula (Ia′)

(Ia′)
(SEQ ID NO: 13)
X1-X2-X3-X4-X5-X6-X7-X8-X9-X10

wherein
X1 is Asp, Glu, Ala, Gly, Thr, Ida, pGlu, bhAsp, D-Asp, Tyr, Leu or absent;
X2 is Thr, Ala, Aib, D-Thr, Arg or absent;
X3 is His, Lys, Ala, or D-His;
X4 is Phe, Ala, Dpa, bhPhe or D-Phe;
X5 is Pro, Glu, Ser, Gly, Arg, Lys, Val, Ala, D-Pro, bhPro, Sarc, Abu or absent;
X6 is IIe, Cys, Arg, Leu, Lys, His, Glu, D-lle, D-Arg, D-Cys, Val, Ser or Ala;
X7 is Cys, IIe, Ala, Leu, Val, Ser, Phe, Dapa, D-Ile or D-Cys;
X8 is IIe, Lys, Arg, Ala, Gin, Phe, Glu, Asp, Tyr, Ser, Leu, Val, D-IIe, D-Lys, D-Arg or Dapa;
X9 is Phe, Ala, IIe, Tyr, Lys, Arg, bhPhe or D-Phe, and
X10 is Lys, Phe or absent;
and provided that if Y′ is absent, X7 is Ile; and
Y′ is a peptide having the formula (IIa′)

(IIa′)
(SEQ ID NO: 16)
Y1-Y2-Y3-Y4-Y5-Y6-Y7-Y8-Y9-Y10-Y11-Y12-Y13-Y14-Y15

wherein
Y1 is Gly, Cys, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp, Ala, His, Tyr or absent;
Y5 is Lys, Met, Arg. Ala or absent;
Y6 is Gly, Ser, Lys, IIe, Arg. Ala, Pro, Val or absent;
Y7 is Trp, Lys, Gly, Ala IIe, Val or absent;
Y8 is Val, Thr, Gly, Cys, Met, Tyr, Ala, Glu, Lys, Asp, Arg or absent;
Y9 is Cys, Tyr or absent;
Y10 is Met, Lys, Arg, Tyr or absent;
Y11 is Arg Met, Cys, Lys or absent;
Y12 is Arg. Lys, Ala or absent;
Y13 is Arg. Cys, Lys, Val or absent;
Y14 is Arg. Lys, Pro, Cys, Thr or absent; and
Y15 is Thr, Arg or absent;
wherein said compound of formula (I) is optionally PEGylated on R¹′, X′, or Y′; and
wherein when said compound of formula (I′) comprises two or more cysteine residues, at least two of said cysteine residues being linked via a disulfide bond.

In some embodiments. R¹′ is hydrogen, isovaleric acid, isobutyric acid or acetyl.

In some embodiments of the peptide compound of formula (I′), X′ is a peptide sequence according to formula (la′), wherein
X1 is Asp, Ala, Ida, pGlu, bhAsp, Leu, D-Asp or absent;
X2 is Thr, Ala, or D-Thr;
X3 is His, Lys, D-His or Lys;
X4 is Phe, Ala, Dpa or D-Phe;
X5 is Pro, Gly, Arg, Lys, Ala, D-Pro or bhPro;
X6 is IIe, Cys, Arg, Lys, D-Ile or D-Cys;
X7 is Cys, IIe, Leu, Val, Phe, D-Ile or D-Cys;
X8 is IIe, Arg, Phe, Gln, Lys, Glu, Val, Leu or D-Ile;
X9 is Phe or bhPhe; and
X10 is Lys, Phe or absent.

In some embodiments of the peptide compound of formula I′, X′ is a peptide sequence having the formula (Ib′)

(Ib′)
(SEQ ID NO: 14)
X1-Thr-His-X4-X5-X6-X7-X8-Phe-X10

wherein
X1 is Asp, Ida, pGlu, bh Asp or absent;
X4 is Phe or Dpa;
X5 is Pro or bhPro;
X6 is IIe, Cys or Arg;
X7 is Cys, IIe, Leu or Val;
X8 is Ile Lys, Glu, Phe, Gin or Arg; and
X10 is Lys or absent.

In some embodiments of the peptide compound of formula I′, X′ is a peptide sequence having the formula (Ic′)

(Ic′)
(SEQ ID NO: 15)
X1-Thr-His-X4-X5-Cys-Ile-X8-Phe-X10

wherein
X1 is Asp, Ida, pGlu, bhAsp or absent;
X4 is: Phe or Dpa;
X5 is Pro or bhPro;
X8 is Ile Lys, Glu, Phe, Gin or Arg; and
X10 is Lys or absent;

In some embodiments of the peptide compound of formula I′, X′ is a peptide sequence having the formula (Id′)

(Id′)
(SEQ ID NO: 4)
X1-Thr-His-Phe-X5-Cys-Ile-X8-Phe-X10

wherein
X1 is Asp, Glu, or Ida;
X4 is Phe;
X5 is Pro or bhPro;
X8 is IIe, Lys, or Phe; and
X10 is absent;

In some embodiments of the peptide compound of formula I′, Y′ is a peptide sequence having the formula (IIb′)

(IIb′)
(SEQ ID NO: 17)
Y1-Y2-Y3-Y4-Y5-Y6-Y7-Y8-Cys-Y10

wherein
Y1 is Gly, Ala, Lys, Pro or D-Pro;
Y2 is Pro, Ala or Gly;
Y3 is Arg, Ala, Lys or Trp;
Y4 is Ser, Gly or Ala;
Y5 is Lys, Met, Arg or Ala;
Y6 is Gly, Arg or Ala;
Y7 is Trp or Ala;
Y8 is Val, Thr, Ala or Glu; and

Y10 is Met, Lys or absent.

In some embodiments of the peptide compound of formula I′, Y′ is a peptide sequence having the formula (IIc′)

(IIc′)
(SEQ ID NO: 18)
Y1-Y2-Y3-Ser-Lys-Gly-Trp-Y8-Cys-Y10

wherein
Y1 is Gly, Pro or D-Pro;
Y2 is Pro or Gly;
Y3 is Arg or Lys,
Y8 is Val or Thr; and
Y10 is Met, Lys or absent.

In some embodiments of the peptide compound of formula I′, Y′ is a peptide sequence having the formula (IId′)

(IId′)
(SEQ ID NO: 19)
Y1-Cys-Y3-Y4-Arg-Y6-Y7-Y8-Cys-Y10-Y11-Y12-Y13-Y14-
Y15

wherein
Y1 is Val or Ala or absent;
Y3 is Gly, Pro or absent;
Y4 is His, Trp or Tyr;
Y6 is Ser, Gly or Pro,
Y7 is IIe, Gly or Lys;
Y8 is Gly, Met or absent;
Y10 is Tyr or Cys;
Y11 is Arg Lys, Met or Ala;
Y12 is Arg or Ala;
Y13 is Cys or Val or absent;
Y14 is Cys, Lys, Pro, Arg. Thr or absent; and
Y15 is Arg. Thr or absent.

In some embodiments of the peptide compound of formula I′, Y′ is a peptide sequence having the formula (IIe′)

(IIe′)
(SEQ ID NO: 20)
Val-Cys-Y3-His-Arg-Y6-Y7-Y8-Cys-Tyr-Arg-Y12-Y13-
Y14-Y15

wherein
Y3 is Gly or absent;
Y6 is Ser or Pro;
Y7 is Ile or Lys;
Y8 is Gly or absent;
Y12 is Arg or Ala;
Y13 is Cys, Val or absent,
Y14 is Cys, Arg. Thr or absent; and
Y15 is Arg or absent.

In some embodiments of the peptide compound of formula I′, Y′ is a peptide sequence having the formula (If)

(IIf′)
(SEQ ID NO: 10)
Y1-Pro-Y3-Ser-Y5-Y6-Y7-Y8-Cys-Y10

wherein
Y1 is Gly, Glu, Val, or Lys;
Y3 is Arg or Lys;
Y5 is Arg or Lys;
Y6 is Gly, Ser, Lys, Ile or Arg;
Y7 is Trp or absent;
Y8 is Val, Thr, Asp, Glu or absent; and
Y10 is Lys or absent.

In some embodiments of the peptide compound of formula I′, Y′ is a peptide sequence having the formula (IIg*)

(IIg′)
(SEQ ID NO: 11)
Y1-Pro-Y3-Ser-Y5-Y6-Y7-Y8-Cys-Y10

wherein
Y1 is Glu or Lys;
Y3 is Arg or Lys;
Y5 is Arg or Lys;
Y6 is Gly, Ser, Lys, Ile or Arg;
Y7 is Trp or absent;
Y8 is Val or absent; and
Y10 is Lys or absent.

In some embodiments, the peptide of formula I′ comprises at least three, at least four, at least five, at least six, at least seven, at least eight, at least nine, at least ten, at least eleven, at least twelve, at least thirteen, at least fourteen or at least fifteen Y residues in Y′,

In some embodiments, Y1 to Y3 are present and Y4 to Y15 are absent.

In some embodiments, Y1 to Y11 are present and Y12 to Y15 are absent.

In some embodiments. Y1 to Y10 are present and Y11 to Y15 are absent.

In some embodiments, Y8 and Y15 are absent.

In some embodiments, Y3 and Y15 are absent. In some embodiments, Y3, Y14 and Y15 are absent

In some embodiment Y5 is absent.

In some embodiments Y1, Y5, Y7, Y12, Y13, Y14 and Y15 are absent.

In some embodiments, the present invention provides a compound of formula (I′), wherein the compound comprises any one of the X′/Y′ peptide sequence formula combinations presented in Table 2 below.

TABLE 2
Illustrative combinations of X′ and Y′
peptides of a compound of Formula (I′)
Formula I′ combinations

| | X′ Peptide | Y′ Peptide |
|---|---|---|
| Combination | Sequence | Sequence |
| Number | Formula | Formula |
| 1 | Ia′ | IIa′ |
| 2 | Ia′ | IIb′ |
| 3 | Ia′ | IIc′ |
| 4 | Ia′ | IId′ |
| 5 | Ia′ | IIe′ |
| 6 | Ia′ | IIf′ |
| 7 | Ia′ | IIg′ |
| 8 | Ib′ | IIa′ |
| 9 | Ib′ | IIb′ |
| 10 | Ib′ | IIc′ |
| 11 | Ib′ | IId′ |
| 12 | Ib′ | IIe′ |
| 13 | Ib′ | IIf′ |
| 14 | Ib′ | IIg′ |
| 15 | Ic′ | IIa′ |
| 16 | Ic′ | IIb′ |
| 17 | Ic′ | IIe′ |
| 18 | Ic′ | IId′ |
| 19 | Ic′ | IIe′ |
| 20 | Ic′ | IIf′ |
| 21 | Ic′ | IIg′ |
| 22 | Id′ | IIa′ |
| 23 | Id′ | IIb′ |
| 24 | Id′ | IIc′ |
| 25 | Id′ | IId′ |
| 26 | Id′ | IIe′ |
| 27 | Id′ | IIf′ |
| 28 | Id′ | IIg′ |

In some embodiments, the invention provides peptides, which may be isolated and/or purified, comprising, consisting essentially of, or consisting of, the following structural formula.

(I″)
(SEQ ID NO: 27)
R¹″-X″-Y″-R²″

or a pharmaceutically acceptable salt or solvate thereof, wherein
R¹″ is hydrogen, an C₁-C₆ alkyl, C₆-C₁₂ aryl, C₆-C₁₂ aryl C₁-C₆ alkyl, C₁-C₂₀ alkanoyl (e.g., methyl, acetyl, formyl, benzoyl or trifluoroacetyl, isovaleric acid, isobutyric acid, octanoic acid, lauric acid and hexadecanoic acid), γ-Glu-hexadecanoic acid) or pGlu, appended to the N-terminus, and including PEGylated versions (e.g., PEG3 to PEG11), alone or as spacers of any of the foregoing;
R²″ is —NH: or —OH,
X″ is a peptide sequence having the formula (Ia″)

(Ia″)
(SEQ ID NO: 22)
X1-X2-X3-X4-X5-X6-X7-X8-X9-X10

wherein
X1 is Asp, Glu, Ala, Gly, Thr, Ida, pGlu, bhAsp, D-Asp. Tyr, Leu or absent;
X2 is Thr, Ala, Aib, D-Thr, Arg or absent;
X3 is His, Lys, Ala, D-His or Lys;
X4 is Phe, Ala, Dpa, bhPhe or D-Phe;
X5 is Pro, Glu, Ser, Gly, Arg, Lys, Val, Ala, D-Pro, bhPro, Sarc, Abu or absent;
X6 is IIe, Cys, Arg, Leu, Lys, His, Glu, D-IIe, D-Arg. D-Cys, Val, Ser or Ala;
X7 is Cys, IIe, Ala, Leu, Val, Ser, Phe, Dapa, D-IIe or D-Cys;
X8 is Ille, Lys, Arg, Ala, Gln, Phe, Glu, Asp, Tyr, Ser, Leu, Val, D-IIe, D-Lys, D-Arg, or Dapa;
X9 is Phe, Ala, IIe, Tyr, Lys, Arg, bhPhe or D-Phe; and
X10 is Lys, Phe or absent;
and provided that if Y″ is absent, X7 is Ile;
wherein said compound of formula I′ is optionally PEGylated on R¹″, X″, or Y″; and
wherein when said compound of formula I″ comprises two or more cysteine residues, at least two of said cysteine residues being linked via a disulfide bond.

In some embodiments, Y″ is absent.

In some embodiments, R¹″ is hydrogen, isovaleric acid, isobutyric acid or acetyl. In some embodiments of the compound of formula (I″), X″ is a peptide sequence according to formula (Ia″), wherein
X1 is Asp, Ala, Ida, pGlu, bhAsp, Leu, D-Asp or absent;
X2 is Thr, Ala, or D-Thr;
X3 is His, Lys, or D-His;
X4 is Phe, Ala, or Dpa;
X5 is Pro, Gly, Arg, Lys, Ala, D-Pro or bhPro;
X6 is IIe, Cys, Arg, Lys, D-Ile or D-Cys;
X7 is Cys, IIe, Leu, Val, Phe, D-Ile or D-Cys;
X8 is IIe, Arg, Phe, Gln, Lys, Glu, Val, Leu or D-Ile;
X9 is Phe or bhPhe; and
X10 is Lys or absent.

In some embodiments of the compound of formula (I″), X″ is a peptide sequence having the formula (Ib″)

(Ib″)
(SEQ ID NO: 23)
X1-Thr-His-X4-X5-X6-X7-X8-Phe-X10

wherein
X1 is Asp, Ida, pGlu, bhAsp or absent;
X4 is Phe or Dpa;
X5 is Pro or bhPro;
X6 is IIe, Cys or Arg,
X7 is Cys, IIe, Leu or Val;
X8 is IIe, Lys, Glu, Phe, Gln or Arg; and
X10 is Lys, Phe or absent.

In some embodiments of the compound of formula (I″), X″ is a peptide sequence having the formula (Ic″)

(Ic″)
(SEQ ID NO: 24)
X1-Thr-His-X4-X5-Cys-Ile-X8-Phe-X10

wherein
X1 is Asp, Ida, pGlu, bhAsp or absent;
X4 is Phe or Dpa:
X5 is Pro or bhPro,
X8 is IIe, Lys, Glu, Phe, Gln or Arg; and
X10 is Lys or absent.

In some embodiments of the compound of formula (I″), X″ is a peptide sequence having the formula (Id″)

(Id″)
(SEQ ID NO: 4)
X1-Thr-His-Phe-X5-Cys-Ile-X8-Phe-X10

wherein
X1 is Asp, Glu or Ida;
X4 is Phe;
X5 is Pro or bhPro;
X8 is IIe, Lys, or Phe; and
X10 is absent.

In some embodiments of the compound of formula (I″), Y″ is a peptide having the formula (IIa″)

(IIa″)
(SEQ ID NO: 25)
Y1-Y2-Y3-Y4-Y5-Y6-Y7-Y8-Cys-Y10

wherein
Y1 is Gly, Ala, Lys, Pro or D-Pro;
Y2 is Pro, Ala or Gly;
Y3 is Arg, Ala, Lys or Trp;
Y4 is Ser, Gly or Ala;
Y5 is Lys, Met, Arg or Ala;
Y6 is Gly, Arg or Ala;
Y7 is Trp Ala or absent;
Y8 is Val, Thr, Lys, Ala, Glu or absent; and
Y10 is Met, Lys or absent.

In some embodiments of the compound of formula (I″), Y″ is a peptide sequence according to formula (IIa″)(SEQ ID NO:25)
wherein
Y1 is Gly, Glu, Val, or Lys
Y2 is Pro
Y3 is Arg or Lys;
Y4 is Ser
Y5 is Arg or Lys,
Y6 is Gly, Ser, Lys, Ile or Arg
Y7 is Trp or absent
Y8 is Val, Thr, Asp, Glu or absent;
Y10 is Lys or absent

In some embodiments of the compound of formula (I″), Y″ is a peptide sequence according to formula (IIa″)(SEQ ID NO:25)
wherein
Y1 is Glo or Lys
Y2 is Pro
Y3 is Arg or Lys,
Y4 is Ser
Y5 is Arg or Lys;
Y6 is Gly, Ser, Lys, Ile or Arg;
Y7 is Trp or absent;
Y8 is Val or absent;
Y10 is Lys or absent

In some embodiments of the compound of formula (I″), Y″ is a peptide sequence according to formula (IIa″)(SEQ ID NO:25)
wherein
Y1 is Gly, Pro or D-Pro.
Y2 is Pro or Gly;
Y3 is Arg or Lys:
Y4 is Ser;
Y5 is Lys:
Y6 is Gly;
Y7 is Trp;
Y8 is Val or Thr; and
Y10 is Met Lys or absent.

In some embodiments of the compound of formula (I″), Y″ is a peptide sequence having the formula (IIb″)

(IIb″)
(SEQ ID NO: 26)
Y1-Y2-Y3-Ser-Lys-Gly-Trp-Y8-Cys-Y10

wherein
Y1 is Gly, Pro or D-Pro;
Y2 is Pro or Gly;
Y3 is Arg or Lys;
Y8 is Val or Thr, and
Y10 is Met, Lys or absent.

In some embodiments, the present invention provides a compound of formula (I), wherein the compound comprises any one of the X″/Y″ peptide sequence formula combinations presented in Table 3 below.

TABLE 3
Illustrative combinations of X″ and Y″
peptides of a compound of Formula (I″)
Formula I″ combinations

| | X″ Peptide | Y″ Peptide |
|---|---|---|
| Combination | Sequence | Sequence |
| Number | Formula | Formula |
| 1 | Ia″ | IIa″ |
| 2 | Ia″ | IIb″ |
| 3 | Ib″ | IIa″ |
| 4 | Ib″ | IIb″ |
| 5 | Ic″ | IIa″ |
| 6 | Ic″ | IIb″ |
| 7 | Id″ | IIa″ |
| 8 | Id″ | IIb″ |

In some embodiments the peptide of formula (I″) comprises at least three, at least four, at least five, at least six, at least seven, at least eight, at least nine, or at least ten Y residues in Y″>In some embodiments, Y I to Y3 are present and Y4 to Y10 are absent. In some embodiments Y5 is absent. In some embodiments Y1, Y5, and Y7 are absent. In some embodiments, Y8 is absent. In some embodiments, Y3 is absent.

In some embodiments, the present invention provides a compound of formula (I), (I′), or (I″), as described herein, each respectively comprising an X, X′, or X″ peptide sequence, according to the present disclosure, wherein X7 is Leu. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I) comprises (a) Ia′, Ib′, Ic′, or Id′ and, optionally, (b) IIa′, IIb′, IIe′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X7 is Val. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I″) comprises (a) Ia′, Ib′, Ie′, or Id′ and, optionally, (b) IIa′, IIb′, IIc″, IId′, IIe′, IIf′, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I″) comprises (a) Ia′, Ib′, Ic′, or Id′ and, optionally, (b) IIa″, IIb′, IIc′, IId′, IIe′, IIf′, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X7 is Cys. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ie′, or Id′ and, optionally, (b) IIa′, IIb′, IIc′, IId′, IIe′, IIf′, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Ile. In particular embodiments, formula (I) comprises (a) Ia, 1b, Ic, or Id and, optionally, (b) IIa, IIb, IIc, IId, IIe, IIf′, or IIg, as described herein. In particular embodiments, formula (I) comprises (a) Ia′, Ib′, Ie′, or Id′ and, optionally, (b) IIa′, IIb′, IIe′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X7 is Ile. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I″) comprises (a) Ia′, Ib′, Ie′, or Id′ and, optionally, (b) IIa′, IIb′, IIc″, IId′, IIe′, IIf′, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (13*), as described herein, each respectively comprising an X, X′, or X″ peptide sequence, according to the present disclosure, wherein X8 is Ile. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I″) comprises (a) Ia′, Ib′, Ic′, or Id′ and, optionally, (b) Ia′, IIb′, IIc′, IId′, IIe′, IIf′, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X8 is Lys. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ie′, or Id′ and, optionally, (b) IIa′, IIb′, IIc′, IId′, IIe′, IIf′, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys and X7 is Ile. In particular embodiments, formula (I) comprises (a) Ia, Ib, Ic, or Id and, optionally, (b) IIa, IIb, IIe, IId, IIe, IIf′, or IIg, as described herein. In particular embodiments, formula (I) comprises (a) Ia′, Ib′, Ic′, or Id′ and, optionally, (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys and X8 is IIe. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ie′, or Id′ and, optionally, (b) IIa′, IIb′, IIc′, IId′, IIe′, IIf′, or IIg′, as described herein. In particular embodiments, formula (I′) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys, X7 is Ille, and X8 is Ile In particular embodiments, formula (I) comprises (a) Ia, Ib, Ic, or Id and, optionally, (b) IIa, IIb, IIc, IId, IIe, IIf, or IIg, as described herein. In particular embodiments, formula (I) comprises (a) Ia, 1b*, Ic′, or Id′ and, optionally. (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Ile and X7 is Cys. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ic′, or Id′ and, optionally, (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X7 is Cys and X8 is Ile. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ich, or Id′ and, optionally, (b) IIa′, IIb′, IIc′, IId′, IIe, IIf″, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is IIe, X7 is Cys, and X8 is Ile. In particular embodiments, formula (I) comprises (a) Ia, Ib, Ic, or Id and, optionally, (b) IIa, IIb, IIc, IId, IIe, IIf, or IIg, as described herein. In particular embodiments, formula (I′) comprises (a) Ia′, Ib′, Ic′, or Id′ and, optionally, (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys and X8 is Lys. In particular embodiments, formula (I) comprises (a) Ia, Ib, Ic, or Id and, optionally, (b) IIa, IIb, IIc, IId, IIe, IIf, or IIg, as described herein. In particular embodiments, formula (I) comprises (a) Ia′, Ib′, Ic′, or Id′ and, optionally, (b) IIa′, IIb′, IIc′, IId′, IIe′, IIf′, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys, X7 is IIe, and X8 is Lys, In particular embodiments, formula (I) comprises (a) Ia, Ib, Ic, or Id and, optionally, (b) IIa, IIb, IIc, IId, IIe, IIf, or IIg, as described herein. In particular embodiments, formula (I) comprises (a) Ia′, Ib″, Ic′, or Id′ and, optionally, (b) IIa′, IIb′, IIc′, IId′, IIe′, IIf′, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys and C7 is Leu. In particular embodiments, formula (I) comprises (a) Ia, Ib, Ic, or Id and, optionally, (b) IIa, IIb, IIc, IId, IIe, IIf, or IIg, as described herein. In particular embodiments, formula (I′) comprises (a) Ia′, Ib′, Ic′, or Id′ and, optionally, (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys and C7 is Val. In particular embodiments, formula (I) comprises (a) Ia, Ib, Ic, or Id and, optionally, (b) IIa, IIb, IIc, IId, IIe, IIf, or IIg, as described herein. In particular embodiments, formula (I′) comprises (a) Ia′, Ib′, Ic′, or Id′ and, optionally, (b) IIa′, IIb′, IIc′, IId′, IIe′, IIf″, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X7 is Ile and X8 is Lys. In particular embodiments, formula (I) comprises (a) Ia, Ib, Ic, or Id and, optionally, (b) IIa, IIb, IIc, IId, IIe, IIf′, or IIg, as described herein. In particular embodiments, formula (P) comprises (a) Ia′, Ib′, Ie′, or Id′ and, optionally, (b) IIa′, IIb′, IIc′, IId′, IIe′, IIf″, or IIg′, as described herein. In particular embodiments, formula (I″) comprises (i) Ia″, Ib″, Ie″, or Id″ and, optionally. (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X7 is Leu and X8 is Lys. In particular embodiments, formula (I) comprises (a) Ia, Ib, Ic, or Id and, optionally, (b) IIa, IIb, IIc, IId, IIe, IIf, or IIg, as described herein. In particular embodiments, formula (I′) comprises (a) Ia′, Ib′,

Ic′, or Id′ and, optionally, (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X7 is Val and X8 is Lys. In particular embodiments, formula (I) comprises (a) Ia, Ib, Ic, or Id and, optionally, (b) IIa, IIb, IIc, IId, IIe, IIf, or IIg, as described herein. In particular embodiments, formula (I′) comprises (a) Ia′, Ib′, Ie′, or Id′ and, optionally, (b) IIa′, IIb′, IIc′, IId′, IIe′, IIf′, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys, X7 is Leu and X8 is Lys. In particular embodiments, formula (I) comprises (a) Ia, Ib, Ic, or Id and, optionally, (b) IIa, IIb, IIc, IId, IIe, IIf, or IIg, as described herein. In particular embodiments, formula (I′) comprises (a) Ia′, Ib*, Ic′, or Id′ and, optionally, (b) IIa′, IIb′, IIc′, IId′, IIe′, IIf′, or IIg*, as described herein. In particular embodiments, formula (I″) comprises (i) Ia″, Ib″, Ic″, or Id″ and, optionally, (if) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys, X7 is Val, and X8 is Lys. In particular embodiments, formula (I) comprises (a) Ia, Ib, Ic, or Id and, optionally, (b) IIa, IIb, IIc, IId, IIe, IIf, or IIg, as described herein. In particular embodiments, formula (I′) comprises (a) Ia′, Ib″, Ic′, or Id′ and, optionally, (b) Ia′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys, X7 is IIe, Leu, or Val. In particular embodiments, formula (I) comprises (a) Ia, Ib, Ic, or Id and, optionally, (b) IIa, IIb, IIc, IId, IIe, IIf, or IIg, as described herein. In particular embodiments, formula (I′) comprises (a) Ia′, Ib′, Ic′, or Id′ and, optionally, (b) Ia′, IIb′, IIc′, IId′, IIe′, IIf′, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys, X7 is IIe, Leu, or Val, and X8 is Lys. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ic′, or Id′ and, optionally, (b) IIa′, IIb′, IIc′, IId′, IIe′, IIf′, or IIg′, as described herein. In particular embodiments, formula (P″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X1 is ASP or IDA, X6 is Cys, X7 is IIe, Leu, or Val, and X8 is Lys. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ic′, or Id′ and, optionally, (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X1 is Asp or IDA, X6 is Cys, X7 is IIe, Len, or Val, and X8 is Lys. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, IIc′, or Id′ and, optionally, (b) IIa′, IIb′, IIc′, IId′, IIe′, IIf′, or IIg′, as described herein. In particular embodiments, formula (I″) comprises (i) Ia″, Ib″, Ic″ or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X2 is Thr, X6 is Cys, X7 is IIe, Leu, or Val, and X8 is Lys. In particular embodiments, formula (I) comprises (a) Ia, Ib, Ic, or Id and, optionally, (b) Ila, IIb, IIc, IId, le, IIf′, or IIg, as described herein. In particular embodiments, formula (I″) comprises (a) Ia′, Ib′, Ic′, or Id′ and, optionally, (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X3 is His, X6 is Cys, X7 is IIe, Leu, or Val, and X8 is Lys. In particular embodiments, formula (I) comprises (a) Ia, Ib, Ic, or Id and, optionally, (b) Ila, IIb, IIc, IId, le, IIf′, or IIg, as described herein. In particular embodiments, formula (I′) comprises (a) Ia′, Ib′, Ic′, or Id′ and, optionally, (b) IIa′, IIb′, IIc′, IId′. IIe′, IIf′, or IIg′, as described herein. In particular embodiments, formula (I″) comprises (i) Ia″, Ib″, Ic″, Id″, and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X4 is Phe, X6 is Cys, X7 is IIe, Leu, or Val, and X8 is Lys. In particular embodiments, formula (I) comprises (a) Ia, Ib, Ic, or Id and, optionally, (b) IIa, IIb, IIe, IId, IIe, IIf′, or IIg, as described herein. In particular embodiments, formula (I) comprises (a) Ia′, Ib′, Ic′, or Id′ and, optionally, (b) IIa′, IIb′, IIe′, IId′, IIe′, III″, or IIg′, as described herein. In particular embodiments, formula (I″) comprises (i) Ia″, Ib″, Ic″, or Id″ and, optionally, (li) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X5 is Pro, X6 is Cys, X7 is IIe, Leu, or Val, and X8 is Lys. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ic′, or Id′ and, optionally, (b) IIa, IIb′, IIc′, IId′, IIe′, If′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys, X7 is IIe, Leu, or Val, X8 is Lys, and X9 is Phe. In particular embodiments, formula (I) comprises (a) Ia, Ib, Ie, or Id and, optionally, (b) Ila, IIb, IIc, IId, IIe, IIf′, or IIg, as described herein. In particular embodiments, formula (I) comprises (a) Ia′, Ib′, Ie′, or Id′ and, optionally, (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X2 is Thr, X6 is Cys, X7 is Ile and X8 is Lys. In particular embodiments, formula (I) comprises (a) Ia, Ib, le, or Id and, optionally, (b) IIa, IIb, IIe, IId, IIe, IIf′, or IIg, as described herein. In particular embodiments, formula (I′) comprises (a) Ia′, Ib′, Ic′, or Id′ and, optionally, (b) IIa′, IIb′, IIc′, IId′, IIe′, IIf′, or IIg′, as described herein. In particular embodiments, formula (P′) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X3 is His, X6 is Cys, X7 is IIe, and X8 is Lys. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ic′, or Id′ and, optionally, (b) IIa′, IIb′, IIc′, IId′, IIe′, IIf″, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I), or (I″), as described herein, each respectively comprising an X, X′, or X″ peptide sequence, according to the present disclosure, wherein X4 is Phe, X6 is Cys, X7 IIe, and X8 is Lys. In particular embodiments, formula (I) comprises (a) Ia, Ib, Ic, or Id and, optionally, (b) IIa, IIb, IIc. IId, IIe, IIf′, or IIg, as described herein. In particular embodiments, formula (I′) comprises (a) Ia′, Ib′, Ic′, or Id′ and, optionally, (b) IIa″, IIb′, IIc′, IId′, IIe′, IIf′, or IIg′, as described herein. In particular embodiments, formula (P″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″peptide sequence, according to the present disclosure, wherein X5 is Pro, X6 is Cys, X7 IIe, and X8 is Lys, In particular embodiments, formula (I) comprises (a) Is, Ib, Ic, or Id and, optionally, (b) IIa, IIb, IIc, IId, IIe, IIf′, or IIg, as described herein. In particular embodiments, formula (I′) comprises (a) Ia′, Ib′, Ic′, or Id′ and, optionally, (b) IIa′, IIb′, IIc′, IId′, IIe′, IIf′, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X6 is Cys, X7 is IIe, X8 is Lys, and X9 is Phe. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ich, or Id′ and, optionally, (b) IIa′, IIb′, IIc′, IId′, IIe′, If, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X5 is Pro, X6 is Cys, X7 is IIe, X8 is Lys, and X9 is Phe. In particular embodiments, formula (I) comprises (a) Ia, Ib, Ie, or Id and, optionally, (b) IIa, IIb, IIc, IId, IIe, IIf′, or IIg, as described herein. In particular embodiments, formula (I′) comprises (a) Ia″, Ib″, IIc′, or Id′ and, optionally, (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (D), (I′), or (I″), as described herein, each respectively comprising an X, X′, or X″ peptide sequence, according to the present disclosure, wherein X4 is Phe, X5 is Pro, X6 is Cys, X7 is IIe, X8 is Lys, and X9 is Phe. In particular embodiments, formula (I) comprises (a) Ia, Ib, le, or Id and, optionally, (b) IIa, IIb, IIc, IId, IIe, IIf′, or IIg, as described herein. In particular embodiments, formula (I′) comprises (a) Ia′, Ib′, IIc′, or Id′ and, optionally, (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X3 is His. X4 is Phe, X5 is Pro, X6 is Cys, X7 is IIe, X8 is Lys, and X9 is Phe. In particular embodiments, formula (I) comprises (a) Ia, Ib, Ic, or Id and, optionally. (b) IIa, IIb, IIc, IId, IIe, IIf′, or IIg, as described herein. In particular embodiments, formula (I″) comprises (a) Ia′, Ib′, Ic′, or Id′ and, optionally, (b) IIa′, IIb′, IIe′, IId′, IIe′, IIf″, or IIg′, as described herein. In particular embodiments, formula (I″) comprises (i) Ia″, Ib″, Ic″, or Id″ and, optionally, (ii) IIa″ or IIb″, as described herein.

In some embodiments, the present invention provides a compound of formula (D), (I′), or (I″), as described herein, each respectively comprising an X, X′, or X″ peptide sequence, according to the present disclosure, wherein X2 is Thr, X3 is His, X4 is Phe, X5 is Pro, X6 is Cys, X7 is IIe, X8 is Lys, and X9 is Phe. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ic′, or Id′ and, optionally, (b) IIa′, IIb′, IIc′, IId′, IIe′, IIf′, or IIg′, as described herein. In particular embodiments, formula (I)) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X1 is Asp or IDA, X2 is Thr, X3 is His, X4 is Phe, X5 is Pro, Xo is Cys, X7 is IIe, X8 is Lys, and X9 is Phe. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ie′, or Id′ and, optionally, (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X1 is Asp or IDA, X2 is Thr, X3 is His, X4 is Phe, X5 is Pro, X6 is Cys, X7 is Ille, Leu, or Val, X8 is Lys, and X9 is Phe. In particular embodiments, formula (I) comprises (a) Ia, Ib, Ic, or Id and, optionally, (b) IIa, IIb, IIc, IId, IIe, IIf, or IIg, as described herein. In particular embodiments, formula (I′) comprises (a) Ia′, Ib′, Ic′, or Id′ and, optionally, (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X1 is Asp, X2 is Thr, X3 is His, X4 is Phe, X5 is Pro X6 is Cys, X7 is IIe, X8 is Lys, and X9 is Phe. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ie′, or Id′ and, optionally, (b) IIa′, IIb′, IIc′, IId′, IIe′, IIf′, or IIg′, as described herein. In particular embodiments, formula (I″) comprises (i) Ia″, Ib″, Ie″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X1 is IDA, X2 is Thr, X3 is His, X4 is Phe, X5 is Pro, X6 is Cys, X7 is IIe, X8 is Lys, and X9 is Phe. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ic′, or Id′ and, optionally, (b) IIa′, IIb′, IIe′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, wherein the compound comprises an R′ that is isovaleric acid. In some embodiments, the present invention provides a compound of formula (I), (I′), or (I″), as described herein, each respectively comprising an X, X′, or X″ peptide sequence, according to the present disclosure, wherein X1 is Asp or IDA, X2 is Thr, X3 is His, X4 is Phe, X5 is Pro, X6 is Cys, X7 is IIe, Leu, or Val, X8 is Lys, and X9 is Phe; wherein said peptide further comprises an R′ that is isovaleric acid. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ic′, or Id′ and, optionally, (b) IIa″, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X1 is Asp, X2 is Thr, X3 is His, X4 is Phe, X5 is Pro, X6 is Cys, X7 is IIe, Lea, or Val, and X8 is Lys; wherein said peptide further comprises an R′ that is isovaleric acid. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I′) comprises (a) Ia′, Ib′, Ie′, or Id′ and, optionally, (b) IIa′, IIb′, IIc′, IId′, IIe′, IIf″, or IIg′, as described herein. In particular embodiments, formula (I″) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X1 is Asp, X2 is Thr, X3 is His, X4 is Phe, X5 is Pro, X6 is Cys, X7 is IIe, Leu, or Val, X8 is Lys, and X9 is Phe; wherein said peptide further comprises an R group that is isovaleric acid. In particular embodiments, formula (I) comprises (a) Ia, Ib, Ic, or Id and, optionally, (b) IIa, IIb, IIc, IId, IIe, IIf′, or IIg, as described herein. In particular embodiments, formula (I) comprises (a) Ia′, Ib′, Ic′, or Id′ and, optionally, (b) IIa′, IIb′, IIc′, IId′, IIe′, IIf′, or IIg′, as described herein. In particular embodiments, formula (I′) comprises (i) Ia″, Ib″, Ic″, or Id″ and, optionally, (ii) IIa″ or IIb′, as described herein.

In some embodiments, the present invention provides a compound of formula (I), (I′), or (I″), as described herein, each respectively comprising an X, X′, or X″ peptide sequence, according to the present disclosure, wherein X1 is Asp, X2 is Thr, X3 is His, X4 is Phe, X5 is Pro, X6 is Cys, X7 is IIe, X8 is Lys, and X9 is Phe; wherein said peptide further comprises an R group that is isovaleric acid. In particular embodiments, formula (I) comprises (a) Ia, 1b, le, or Id and, optionally, (b) IIa, IIb, IIc, IId, IIe, IIf′, or IIg, as described herein. In particular embodiments, formula (I′) comprises (a) Ia′, Ib′, Ic′, or Id′ and, optionally, (b) IIa′, IIb′, IIc′, IId′, IIe′, IIf″, or IIg′, as described herein. In particular embodiments, formula (I″) comprises (i) Ia″, Ib″, Ic”, or Id″ and, optionally, (ii) IIa″ or IIb′, as described herein.

In some embodiments, the present invention provides a compound of formula (I), (I′), or (I″), as described herein, wherein the compound comprises a peptide sequence that is 85% or higher (e.g. 85%, 90%, 91%, 92%, 93%, 94%, 95%, 96%, 97%, 98%, 99%, or 99.5%) homologous to an amino acid sequence set forth in any one of Tables 5-15. In particular embodiments, formula (I) comprises (a) Ia, Ib. Ic, or Id and, optionally, (b) IIa, IIb, IIc, IId, IIe, If, or IIg, as described herein. In particular embodiments, formula (I) comprises (a) Ia′, Ib′. Ic′, or Id′ and, optionally. (b) IIa′, IIb′, IIc′, IId′, IIe′, IIf′, or IIg′, as described herein. In particular embodiments, formula (I″) comprises (i) Ia″, Ib″, Ic″, or Id″ and, optionally, (ii) IIa″ or IIb′, as described herein.

In certain embodiments, a peptide or a peptide dimer of the present invention comprises any one of the compounds shown in any one of Tables 5-15.

In certain embodiments, a peptide or a peptide dimer of the present invention comprises any one of the amino acid sequences provided as SEQ ID NOS: 1-334 and 338-375, or as shown in any one of Tables 5-15

In certain embodiments, a peptide or a peptide dimer of the present invention comprises an amino acid sequence set forth in any one of Tables 5-15.

In certain embodiments, a peptide or a peptide dimer of the present invention has a structure shown in any one of Tables 5-15, e.g., Tables 7 or 12-15. In one certain embodiment, a peptide or a peptide dimer of the present invention comprises an amino acid sequence set forth in any one of Tables 5-15, e.g., Tables 7 or 12-15. In some embodiments, a peptide of the present invention comprises an amino acid sequence having at least about 85% identical or at least about 90%, 95%, 97%, 98%, 99% identical to any amino acid sequence set forth in any one of Tables 5-15, e.g., Tables 7 or 12-15, or any one of SEQ ID NOS: 1-334 and 338-375. In one certain embodiment, a peptide or a peptide dimer of the present invention comprises an amino acid sequence having at least about 85% identical or at least about 90%, 95%, 97%, 98%, 99% identical to any amino acid sequence set forth in Table 7 or any one of Tables 5-15

It is understood that in the context of the invention, a peptide or peptide dimer comprising a peptide sequence shown in one of the accompanying Tables or sequence listing may have certain minor alterations to one or more amino acid residues of the peptide sequence, as compared to the native amino acid, yet still be considered to comprises the peptide sequence shown in the Tables or sequence listing. For example, one or more side chains of one or more amino acid residues present in the peptide or peptide dimer may be slightly altered due to the attachment of a linker or dimerization via cysteine residues, or an N-terminal or C-terminal amino acid may be amidated.

In some embodiments, a peptide or a peptide dimer of the present invention exhibits hepcidin activity. In some embodiments, a peptide or a peptide dimer of the present invention exhibits at least about 50%, 60%, 70%, 80%, 90%, 95%, 97%, 98%, 99%, greater than 99%, greater than 100%, greater than 110%, greater than 120%, greater than 150%, greater than 200% greater than 500%, or greater than 1000% of the activity of a reference hepcidin, (e.g., any one of the hepcidin reference compounds provided in Table 4). In some embodiments, the activity is an in vitro or an in vivo activity as described herein.

In some embodiments, a peptide or a peptide dimer of the present invention exhibits at least about 50%, 60%, 70%, 80%, 90%, 95%, 97%, 98%, 99%, or greater than 99% of the in vitro activity for inducing the degradation of human ferroportin protein as that of a reference hepcidin, (e.g., any one of the hepcidin reference compounds provided in Table 4), wherein the activity is measured according to the methods described herein (e.g., according to Example 2).

In some embodiments, a peptide or a peptide dimer of the present invention exhibits at least about 50%, 60%, 70%, 80%, 90%, 95%, 97%, 98 99%, or greater than 99% of the in vivo activity for inducing the reduction of free plasma iron in an individual as does a reference hepcidin, (e.g., any one of the hepcidin reference compounds provided in Table 4), wherein the activity is measured according to the methods described herein (e.g., according to Example 8).

In some embodiments, a peptide or a peptide dimer of the present invention exhibits increased hepcidin activity as compared to a hepcidin reference peptide, (e.g., any one of the hepcidin reference compounds provided in Table 4). In certain embodiments, a peptide or a peptide dimer of the present invention exhibits 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, 500%, 700%, or 1000% greater activity than a reference hepcidin, (e.g., any one of the hepcidin reference compounds provided in Table 4). In some embodiments, a peptide or a peptide dimer of the present invention exhibits at least about 50%, 60%, 70%, 80%, 90%, 95%, 97%, 98%, 99%, or greater than 99% of the activity exhibited by a hepcidin reference compound. In some embodiments, the activity is an in vitro or an in vivo activity, e.g., an in vivo or an in vitro activity described herein. In certain embodiments, a peptide or a peptide dimer of the present invention exhibits 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, 500%, 700%, or 1000% greater activity than a reference hepcidin, (e.g., any one of the hepcidin reference compounds provided in Table 4), wherein the activity is an in vitro activity for inducing the degradation of ferroportin, e.g., as measured according to Example 2; or wherein the activity is an in vivo activity for reducing free plasma iron, e.g., as measured according to Example 8.

In some embodiments, a peptide or a peptide dimer of the present invention binds ferroportin, e.g., human ferroportin. In some embodiments, a peptide or a peptide dimer of the present invention exhibits at least about 50%, 60%, 70%, 80%, 90%, 95%, 97%, 98%, 99%, or greater than 99% of the ferroportin binding ability that is exhibited by a reference hepcidin (e.g., any one of the hepcidin reference compounds provided in Table 4). In some embodiments, a peptide or a peptide dimer of the present invention has a lower IC₅₀ (i.e., higher binding affinity) for binding to ferroportin, (e.g., human ferroportin) compared to a reference hepcidin, (e.g., any one of the hepcidin reference compounds provided in Table 4). In some embodiments, the peptide of the present invention has an IC₅₀ in a ferroportin competitive binding assay which is at least about 10%, 20%, 30%, 40%, 50%, 60%, 70%, 80%, 90%, 100%, 200%, 300%, 400%, 500%, 700%, or 1000% lower than a reference hepcidin, (e.g., any one of the hepcidin reference compounds provided in Table 4).

In some embodiments, the present invention provides a compound of formula I, P′, or I′, as described herein, wherein the peptide exhibits increased stability (e.g., as measured by half-life, rate of protein degradation) as compared to a reference hepcidin, (e.g., any one of the hepcidin reference compounds provided in Table 4). In some embodiments, the present invention provides a dimer of such a compound, and in certain embodiments the dimer is a homodimer. In certain embodiments, the stability of a peptide or a peptide dimer of the present invention is increased at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, or 500% greater than a reference hepcidin, (e.g., any one of the hepcidin reference compounds provided in Table 4). In some embodiments, the stability is a stability that is described herein. In some embodiments, the stability is a plasma stability, e.g., as optionally measured according to the method described in Example 7.

In particular embodiments, the present invention provides a compound of formula I, I′, or I″, as described herein, wherein the peptide exhibits a longer half-life than a reference hepcidin, (e.g., any one of the hepcidin reference compounds provided in Table 4). In some embodiments, the present invention provides a dimer of such a compound, and in certain embodiments the dimer is a homodimer. In particular embodiments, a peptide or a peptide dimer of the present invention has a half-life under a given set of conditions (e.g., temperature, pH) of at least about 5 minutes, at least about 10 minutes, at least about 20 minutes, at least about 30 minutes, at least about 45 minutes, at least about 1 hour, at least about 2 hour, at least about 3 hours, at least about 4 hours, at least about 5 hours, at least about 6 hours, at least about 12 hours, at least about 18 hours, at least about 1 day, at least about 2 days, at least about 4 days, at least about 7 days, at least about 10 days, at least about two weeks, at least about three weeks, at least about 1 month, at least about 2 months, at least about 3 months, or more, or any intervening half-life or range in between, about 5 minutes, about 10 minutes, about 20 minutes, about 30 minutes, about 45 minutes, about 1 hour, about 2 hour, about 3 hours, about 4 hours, about 5 hours, about 6 hours, about 12 hours, about 18 hours, about 1 day, about 2 days, about 4 days, about 7 days, about 10 days, about two weeks, about three weeks, about 1 month, about 2 months, about 3 months, or more, or any intervening half-life or range in between. In some embodiments, the half life of a peptide or & peptide dimer of the present invention is extended due to its conjugation to one or more lipophilic substituent, e.g., any of the lipophilic substituents disclosed herein. In some embodiments, the half life of a peptide or a peptide dimer of the present invention is extended due to its conjugation to one or more polymeric moieties, e.g., any of the polymeric moieties disclosed herein. In certain embodiments, the temperature is about 25° C., about 4° C., or about 37° C., and the pH is a physiological pH, or a pH about 7.4.

In some embodiments, the half-life is measured in vitro using any suitable method known in the art, e.g., in some embodiments, the stability of a peptide or a peptide dimer of the present invention is determined by incubating the peptide or the peptide dimer with pre-warmed human serum (Sigma) at 37° C. Samples are taken at various time points, typically up to 24 hours, and the stability of the sample is analyzed by separating the peptide or peptide dimer from the serum proteins and then analyzing for the presence of the peptide or peptide dimer of interest using LC-MS.

In some embodiments, the stability of the peptide is measured in vivo using any suitable method known in the art, e.g., in some embodiments, the stability of a peptide or a peptide dimer is determined in vivo by administering the peptide or peptide dimer to a subject such as a human or any mammal (e.g., mouse) and then samples are taken from the subject via blood draw at various time points, typically up to 24 hours. Samples are then analyzed as described above in regard to the in vitro method of measuring half-life. In some embodiments, in vivo stability of a peptide or a peptide dimer of the present invention is determined via the method disclosed in Example 7.

In some embodiments, the present invention provides a compound of formula I, I′, or I″, as described herein, or a dimer thereof, wherein the peptide or the dimer exhibits improved solubility or improved aggregation characteristics as compared to a reference hepcidin, (e.g., any one of the hepcidin reference compounds provided in Table 4). Solubility may be determined via any suitable method known in the art. In some embodiments, suitable methods known in the art for determining solubility include incubating peptides in various buffers (Acetate pH4.0, Acetate pH 5.0, Phos/Citrate pH 5.0, Phos Citrate pH6.0, Phos pH 6.0, Phos pH 7.0, Phos pH7.5, Strong PBS pH 7.5, Tris pH7.5, Tris pH 8.0, Glycine pH 9.0, Water, Acetic acid (pH 5.0 and other known in the art) and testing for aggregation or solubility using standard techniques. These include, but are not limited to, visual precipitation, dynamic light scattering, Circular Dichroism and fluorescent dyes to measure surface hydrophobicity, and detect aggregation or fibrillation, for example. In some embodiments, improved solubility means the peptide is more soluble in a given liquid than is a reference hepcidin (e.g., any one of the hepcidin reference compounds provided in Table 4).

In some embodiments, the present invention provides a compound of formula I, I′, or I″, as described herein, or a dimer thereof, wherein the peptide or the dimer exhibits less degradation (i.e., more degradation stability), e.g., greater than or about 10% less, greater than or about 20% less, greater than or about 30% less, greater than or about 40 less, or greater than or about 50% less than a reference hepcidin (e.g., any one of the hepcidin reference compounds provided in Table 4). In some embodiments, degradation stability is determined via any suitable method known in the art. In some embodiments, suitable methods known in the art for determining degradation stability include the method described in Hawe et al J Pharm Sci, VOL. 101, NO. 3, 2012, p 895-913, incorporated herein in its entirety. Such methods are in some embodiments used to select potent sequences with enhanced shelf lives.

In some embodiments, the present invention provides compositions and medicaments comprising at least one peptide or peptide dimer as disclosed herein. In some embodiments, the present invention provides a method of manufacturing medicaments comprising at least one peptide or peptide dimer as disclosed herein for the treatment of diseases of iron metabolism, such as iron overload diseases. In some embodiments, the present invention provides a method of manufacturing medicaments comprising at least one peptide or peptide dimer as disclosed herein for the treatment of diabetes (Type I or Type II), insulin resistance, or glucose intolerance. Also provided are methods of treating a disease of iron metabolism in a subject, such as a mammalian subject, and preferably a human subject, comprising administering at least one peptide, peptide dimer, or composition as disclosed herein to the subject. In some embodiments, the peptide, peptide dimer, or the composition is administered in a therapeutically effective amount. Also provided are methods of treating diabetes (Type I or Type II), insulin resistance, or glucose intolerance in a subject, such as a mammalian subject, and preferably a human subject, comprising administering at least one peptide, peptide dimer, or composition as disclosed herein to the subject. In some embodiments, the peptide, peptide dimer, or composition is administered in a therapeutically effective amount.

In some embodiments, the peptide, or peptide dimer of this invention is synthetically manufactured. In other embodiments, the peptide or peptide dimer of this invention is recombinantly manufactured.

In some embodiments, the invention provides a process for manufacturing a compound, peptide, peptide analogue, peptide dimer, or pharmaceutical composition as disclosed herein.

In some embodiments, the invention provides a device comprising at least one peptide, peptide analogue, or peptide dimer of the present invention, or pharmaceutically acceptable salt or solvate thereof for delivery of the peptide analogue or the peptide dimer to a subject.

In some embodiments, the present invention provides methods of binding a ferroportin or inducing ferroportin internalization and degradation which comprises contacting the ferroportin with at least one peptide or peptide analogue, peptide dimer, or composition as disclosed herein.

In some embodiments, the present invention provides kits comprising at least one peptide, peptide analogue, peptide dimer, or composition as disclosed herein packaged together with a reagent, a device, instructional material, or a combination thereof.

In some embodiments, the present invention provides complexes which comprise at least one peptide or peptide dimer as disclosed herein bound to a ferroportin, preferably a human ferroportin, or an antibody, such as an antibody which specifically binds a peptide or a peptide dimer as disclosed herein, Hep25, or a combination thereof.

In some embodiments, the compound has a measurement (e.g., an EC50) of less than 500 nM within the Fpn internalization assay. As a skilled person will realize, the function of the peptide is dependent on the tertiary structure of the peptide and the binding surface presented. It is then possible to make minor changes of the sequence that do not affect the fold or are not on the binding surface and maintain function. In other embodiments, the compound of the invention is a peptide or peptidomimetic compound, or a dimer thereof having 85% or higher (e.g., 85%, 90%, 91%, 92%, 93%, 94%, 95%, 96%, 97%, 98%, 99%, or 99,5%) identity or homology to an amino acid sequence of any compound of formula I, I′, or I″ that exhibits an activity, or lessens a symptom of a disease or indication for which hepcidin is involved.

In some embodiments, the peptide, peptide analogue, or dimer thereof of the invention may comprise functional fragments of variants thereof that have at most 10, 9, 8, 7, 6, 5, 4, 3, 2, or 1 amino acid substitutions compared to one or more of the specific sequences recited below.

In addition to the methods disclosed herein in Example 1, the peptides and the peptide dimers of the present invention may be produced using methods known in the art including chemical synthesis, biosynthesis or in vitro synthesis using recombinant DNA methods, and solid phase synthesis. See e.g. Kelly & Winkler (1990) Genetic Engineering Principles and Methods, vol. 12, J. K. Setlow ed., Plenum Press, NY, pp. 1-19; Merrifield (1964) J Amer Chem Soc 85:2149; Houghten (1985) PNAS USA 82:5131-5135; and Stewart & Young (1984) Solid Phase Peptide Synthesis, 2ed. Pierce, Rockford, IL, which are herein incorporated by reference. The peptides of the present invention may be purified using protein purification techniques known in the art such as reverse phase high-performance liquid chromatography (HPLC), ion-exchange of immunoaffinity chromatography, filtration or size exclusion, or electrophoresis. See Olsnes, S. and A. Pihl (1973) Biochem. 12 (16): 3121-3126; and Scopes (1982) Protein Purification, Springer-Verlag, NY, which are herein incorporated by reference. Alternatively, the peptides of the present invention may be made by recombinant DNA techniques known in the art. Thus, polynucleotides that encode the polypeptides of the present invention are contemplated herein. In preferred embodiments, the polynucleotides are isolated. As used herein “isolated polynucleotides” refers to polynucleotides that are in an environment different from that in which the polynucleotide naturally occurs.

In certain embodiments, peptides of the present invention bind ferroportin, preferably human ferroportin. Preferred peptides of the present invention specifically bind human ferroportin. As used herein, “specifically binds” refers to a specific binding agent's preferential interaction with a given ligand over other agents in a sample. For example, a specific binding agent that specifically binds a given ligand, binds the given ligand, under suitable conditions, in an amount or a degree that is observable over that of any nonspecific interaction with other components in the sample. Suitable conditions are those that allow interaction between a given specific binding agent and a given ligand. These conditions include pH, temperature, concentration, solvent, time of incubation, and the like, and may differ among given specific binding agent and ligand pairs, but may be readily determined by those skilled in the art.

The peptides of the present invention that mimic the hepcidin activity of Hep25, the bioactive human 25-amino acid form, are herein referred to as “mini-hepcidins”. As used herein, in certain embodiments, a compound having “hepcidin activity” means that the compound has the ability to lower plasma iron concentrations in subjects (e.g. mice or humans), when administered thereto (e.g. parenterally injected or orally administered), in a dose-dependent and time-dependent manner. See e.g. as demonstrated in Rivera et al. (2005), Blood 106:2196-9. In some embodiments, the peptides of the present invention lower the plasma iron concentration in a subject by at least about 1.2, 1.5, 2, 3, 4, 5, 6, 7, 8, 9, or 10-fold, or at least about 5%, 10%, 20%, 25%, 30%, 40%, 50%, 60%, 70%, 80%, 90%, or about 99%.

In some embodiments, the peptides of the present invention have in vitro activity as assayed by the ability to cause the internalization and degradation of ferroportin in a ferroportin-expressing cell line as taught in Nemeth et al. (2006) Blood 107:328-33. In vitro activity may be measured by the dose-dependent loss of fluorescence of cells engineered to display ferroportin fused to green fluorescent protein as in Nemeth et al. (2006) Blood 107:328-33. Aliquots of cells are incubated for 24 hours with graded concentrations of a reference preparation of Hep25 or a mini-hepcidin. As provided herein, the EC50 values are provided as the concentration of a given compound (e.g. peptide) that elicits 50% of the maximal loss of fluorescence generated by the reference Hep25 preparation. EC50 of Hep25 preparations in this assay range from 5 to 15 nM and preferred mini-hepcidins have EC50 values in in vitro activity assays of about 1,000 nM or less. In certain embodiments, a peptide of the present invention has an EC50 in an in vitro activity assay (e.g., as described in Nemeth et al. (2006) Blood 107:328-33 or Example 2 herein) of less than about any one of 0.01, 0.05, 0 1, 0.2, 0.3, 0 4, 0.5, 0.6, 0. 7, 0.8, 0 9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, a peptide analogue or biotherapeutic composition has an EC₅₀ value of about 1 nM or less.

Other methods known in the art for calculating the hepcidin activity and in vitro activity of peptides according to the present invention may be used. For example, the in vitro activity of compounds may be measured by their ability to internalize cellular ferroportin, which is determined by immunohistochemistry or flow cytometry using antibodies which recognizes extracellular epitopes of ferroportin. Alternatively, the in vitro activity of compounds may be measured by their dose-dependent ability to inhibit the efflux of iron from ferroportin-expressing cells that are preloaded with radioisotopes or stable isotopes of iron, as in Nemeth et al. (2006) Blood 107:328-33.

### Conjugation

The skilled person will be well aware of suitable techniques for preparing the compounds employed in the context of the invention. For examples of suitable chemistry, see, e.g., WO98/08871, WO00/55184, WO00/55119. Madsen et al (J. Med. Chem. 2007, 50, 6126-32), and Knudsen et al. 2000 (J. Med Chem. 43, 1664-1669).

The side chains of one or more amino acid residues (e.g. Lys residues) in a compound of the invention may be further conjugated (i.e. covalently attached) to a lipophilic substituent. The lipophilic substituent may be covalently bonded to an atom in the amino acid side chain, or alternatively may be conjugated to the amino acid side chain via one or more spacers. The amino acid(s) in question may be part of the peptide moiety X, or a part of the peptide moiety Y.

Without wishing to be bound by any particular theory, itis believed that the lipophilic substituent binds to albumin in the blood stream, thereby shielding the peptide analogue of the invention from enzymatic degradation, and thus enhancing its half-life. The spacer, when present, may provide spacing between the peptide analogue and the lipophilic substituent.

In certain embodiments, the lipophilic substituent may comprise a hydrocarbon chain having from 4 to 30 C atoms, for example at least 8 or 12 C atoms, and preferably 24 C atoms or fewer, or 20 C atoms or fewer. The hydrocarbon chain may be linear or branched and may be saturated or unsaturated. In certain embodiments, the hydrocarbon chain is substituted with a moiety which forms part of the attachment to the amino acid side chain or the spacer, for example an acyl group, a sulfonyl group, an N atom, an O atom or an S atom. In some embodiments, the hydrocarbon chain is substituted with an acyl group, and accordingly the hydrocarbon chain may form part of an alkanoyl group, for example palmitoyl, caproyl, lauroyl, myristoyl or stearoyl.

A lipophilic substituent may be conjugated to any amino acid side chain in a compound of the invention. In certain embodiment, the amino acid side chain includes a carboxy, hydroxyl, thiol, amide or amine group, for forming an ester, a sulphonyl ester, a thioester, an amide or a sulphonamide with the spacer or lipophilic substituent. For example, the lipophilic substituent may be conjugated to Asn, Asp, Glu, Gln, His, Lys, Arg, Ser, Thr, Tyr, Trp, Cys or Dbu, Dpr or Orn. In certain embodiments, the lipophilic substituent is conjugated to Lys. An amino acid shown as Lys in any of the formulae provided herein may be replaced by, e.g., Dbu, Dpr or Orn where a lipophilic substituent is added

In further embodiments of the present invention, alternatively or additionally, the side-chains of one or more amino acid residues in the compound of the invention may be conjugated to a polymeric moiety, for example, in order to increase solubility and/or half-life in vivo (e.g. in plasma) and/or bioavailability. Such modifications are also known to reduce clearance (e.g. renal clearance) of therapeutic proteins and peptides.

As used herein, “Polyethylene glycol” or “PEG” is a polyether compound of general formula H—(O—CH2—CH2)ₙ—OH. PEG; are also known as polyethylene oxides (PEOs) or polyoxyethylenes (POEs), depending on their molecular weight PEO, PEE, or POG, as used herein, refers to an oligomer or polymer of ethylene oxide. The three names are chemically synonymous, but PEG has tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. Throughout this disclosure, the 3 names are used indistinguishably. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g. viscosity) due to chain length effects, their chemical properties are nearly identical. The polymeric moiety is preferably water-soluble (amphiphilic or hydrophilic), non-toxic, and pharmaceutically inert. Suitable polymeric moieties include polyethylene glycols (PEG), homo- or co-polymers of PEG, a monomethyl-substituted polymer of PEG (mPEG), or polyoxyethylene glycerol (POG). See, for example, Int. J. Hematology 68:1 (1998); Bioconjugate Chem. 6:150 (1995); and Crit. Rev. Therap. Drug Carrier Sys. 9:249 (1992). Also encompassed are peptides that are prepared for purpose of half life extension, for example, mono-activated, alkoxy-terminated polyalkylene oxides (POA's) such as mono-methoxy-terminated polyethyelene glycols (mPEG's); bis activated polyethylene oxides (glycols) or other PEG derivatives are also contemplated. Suitable polymers will vary substantially by weights ranging from about 70 to about 40,000 or from about 200 to about 40,000 are usually selected for the purposes of the present invention. Molecular weights from 200 to 2,000 are preferred and 200 to 500 are particularly preferred. There are different forms of PEG are also available, depending on the initiator used for the polymerization process—the most common initiator is a monofunctional methyl ether PEG, or methoxypoly(ethylene glycol), abbreviated mPEG.

As used herein, lower-molecular weight PEGs are also available as pure oligomers, referred to as monodisperse, uniform, or discrete. These are used in certain embodiments of the present invention.

PEGs are also available with different geometries: Branched PEGs have three to ten PEG chains emanating from a central core group; Star PEGs have 10 to 100 PEG chains emanating from a central core group; Comb PEGs have multiple PEG chains normally grafted onto a polymer backbone. PEGs can also be linear. The numbers that are often included in the names of PEGs indicate their average molecular weights (e.g. a PEG with n=9 would have an average molecular weight of approximately 400 daltons, and would be labeled PEG 400.

As used herein, “PEGylation” is the act of covalently coupling a PEG structure to the peptide of the invention, which is then referred to as a “PEGylated peptide”. In some embodiments, the X moiety of formula I, the Y moiety of formula I, the R′ moiety of formula I, the R′ moiety of formula I, or any combination thereof, is PEGylated. In some embodiments, the X′ moiety of formula I′, the Y′ moiety of formula I′, the RI moiety of formula I′, the R²′moiety of formula I′, or any combination thereof, is PEGylated. In some embodiments, the X″ moiety of formula I″, the Y″ moiety of formula I″, the R″>moiety of formula I″, the R²″>moiety of formula 1″, or any combination thereof, is PEGylated. In some embodiments, one or more side chains of an amino acid in the peptide of formula I, formula I′, or formula P″ is PEGylated. In certain embodiments, the PEG of the PEGylated side chain is a PEG with a molecular weight from about 200 to about 40,000. In some embodiments, a spacer of a peptide of formula I, formula I′, or formula I″ is PEGylated. In certain embodiments, the PEG of a PEGylated spacer is PEG3, PEG4, PEG5, PEG6, PEG7, PEG8, PEG9, PEG10, or PEG11. In certain embodiments, the PEG of a PEGylated spacer is PEG3 or PEGS. In certain embodiments, the PEG of a PEGylated spacer is PEG3 or PEG8.

Other suitable polymeric moieties include poly-amino acids such as poly-lysine, poly-aspartic acid and poly-glutamic acid (see for example Gombotz, et al. (1995), Bioconjugate Chem., vol. 6:332-351; Hudecz, et al. (1992), Bioconjugate Chem., vol. 3, 49-57 and Tsukada, et al. (1984), J. Natl. Cancer Inst., vol. 73: 721-729. The polymeric moiety may be straight-chain or branched. In some embodiments, it has a molecular weight of 500-40,000 Da, for example 500-10,000 Da, 1000-5000 Da, 10,000-20,000 Da, or 20,000-40,000 Da.

In some embodiments, a compound of the invention may comprise two or more such polymeric moieties, in which case the total molecular weight of all such moieties will generally fall within the ranges provided above.

In some embodiments, the polymeric moiety may be coupled (by covalent linkage) to an amino, carboxyl or thiol group of an amino acid side chain. Preferred examples are the thiol group of Cys residues and the epsilon amino group of Lys residues, and the carboxyl groups of′ Asp and Glu residues may also be involved.

The skilled worker will be well aware of suitable techniques which can be used to perform the coupling reaction. For example, a PEG moiety bearing a methoxy group can be coupled to a Cys thiol group by a maleimido linkage using reagents commercially available from Nektar Therapeutics AL. See also WO 2008/101017, and the references cited above, for details of suitable chemistry. A maleimide-functionalised PEG may also be conjugated to the side-chain sulfhydryl group of a Cys residue.

As used herein, disulfide bond oxidation can occur within a single step or is a two step process. As used herein, for a single oxidation step the trityl protecting group is often employed during assembly, allowing deprotection during cleavage, followed by solution oxidation. When a second disulfide bond is required one has the option of native or selective oxidation. For selective oxidation requiring orthogonal protecting groups Acm and Trityl is used as the protecting groups for cysteine. Cleavage results in the removal of one protecting pair of cysteine allowing oxidation of this pair. The second oxidative deprotection step of the cysteine protected Acm group is then performed. For native oxidation the trityl protecting group is used for all cysteines, allowing for natural folding of the peptide.

A skilled worker will be well aware of suitable techniques which can be used to perform the oxidation step.

### Peptide Dimers

The term “dimer,” as in a peptide dimer, refers to compounds in which two peptide chains are linked, either identical (homo-dimer) or non-identical (hetero-dimer) through a linking moiety. A cysteine dimer is then two peptides chains linked through the amino acid cysteine disulfide bond.

In some embodiments, the peptides of the present invention may be active in a dimer conformation or a hetero-dimer conformation, in particular when free cysteine residues are present in the peptide. In certain embodiments, this occurs either as a synthesized dimer or, in particular, when a free cysteine monomer peptide is present and under oxidizing conditions, dimerizes. In some embodiments, the dimer is a homodimer. In other embodiments, the dimer is a heterodimer.

In certain embodiments, a peptide analogue of the present invention is a peptide dimer comprising a peptide of the invention. In particular embodiments, the peptide dimers comprise a peptide of formula I, a peptide of formula I′, or a peptide of formula I″. In particular embodiments, the peptide dimers comprise two peptides of formula I, two peptides of formula I′, or two peptides of formula I″. In certain embodiments, the peptide dimers are homodimers. In particular embodiments wherein the peptide dimer comprises a peptide of formula I, X has the formula Ia, Ib, Ic, or Id. In particular embodiments wherein the peptide dimer comprises a peptide of formula I, Y has the formula IIa, IIb, IIc, IId, IIe, IIf′, or IIg. In particular embodiments wherein the peptide dimer comprises a peptide of formula P′, X′ has the formula Ia′, Ib′, Ic′, or Id′. In particular embodiments wherein the peptide dimer comprises a peptide of formula I′, Y′ has the formula IIa′, IIb′, IIe′, IId′, IIe′, IIf′, or IIg′. In particular embodiments wherein the peptide dimer comprises a peptide of formula I′, X″ has the formula Ia″, Ib″, Ic “; or Id”. In particular embodiments wherein the peptide dimer comprises a peptide of formula I″, Y″ has the formula IIa″ or IIb″.

In some embodiments, the dimer is between two X groups of formula I, two X′ groups of formula I′, or two X″ groups of formula I″, e.g., the two peptides of the dimer are linked through two X groups of formula I, two X′ groups of formula I′, or two X″ groups of formula I″. In some embodiments, the dimer comprises two X groups of formula I, two X′ groups of formula I′, or two X″ groups of formula I″. In some embodiments, the two X groups, X″ groups, or X″ groups in the dimers comprise the same amino acid residues. In some embodiments, the two X groups, X′ groups, or X″ groups in the dimers comprise different amino acid residues (i.e., each amino acid in each of the two X, X′ or X″ groups is independently selected). In some embodiments, the dimer is between two Y groups of formula I, two Y groups of formula I′, or two Y″ groups of formula I″, e.g., the two peptides of the dimer are linked through two Y groups of formula I, two Y′ groups of formula I′, or two Y″ groups of formula I″. In some embodiments, the dimer comprises two Y groups of formula I, two Y groups of formula P′, or two Y″ groups of formula I″. In some embodiments, the two Y groups, Y′ groups, or Y″ groups in the dimer comprise the same amino acid residues. In some embodiments, the two Y groups, Y′ groups or Y″ groups in the dimer comprise different amino acid residues (i.e., each amino acid in each of the Y, Y′ or Y″ groups is independently selected). In some embodiments, a dimer is between an X group of formula I and a Y group of formula I (e.g., the two peptides of the dimer are linked through an X group of formula I and a Y group of formula I), an X′ group of formula I′ and a Y′ group of formula I (e.g., the two peptides of the dimer are linked through an X′ group of formula I′ and a Y′ group of formula l′), or an X″ group of formula I″ and a Y″ group of formula I″ (e.g., the two peptides of the dimer are linked through an X″ group of formula I′ and a Y″ group of formula I″).

In particular embodiments, a peptide dimer of the present invention comprises a peptide comprising: a peptide sequence set forth in any one of Tables 5-15 or SEQ ID NOs: 1-334 and 338-375; or a peptide sequence having at least 85%, at least 90%, at least 91%, at least 92%, at least 93%, at least 94%, at least 95%, at least 96%, at least 97%, at least 98%, or at least 99% identity to a peptide sequence set forth in any one of Tables 5-15 or SEQ ID NOs: 1-334 and 338-375. In particular embodiments, a peptide dimer of the present invention is a homodimer comprising two peptides, each comprising: a peptide sequence set forth in any one of Tables 5-15 or SEQ ID NOs: 1-334 and 338-375; or a peptide sequence having at least 85%, at least 90%, at least 91%, at least 92%, at least 93%, at least 94%, at least 95%, at least 96%, at least 97%, at least 98%, or at least 99% identity to a peptide sequence set forth in any one of Tables 5-15 or SEQ ID NOs: 1-334 and 338-375. In particular embodiments, a peptide dimer of the present invention comprises a compound set forth in any one of Tables 5-15. In particular embodiments, a peptide dimer of the present invention is a homodimer comprising two peptides, each comprising a compound set forth in any one of Tables 5-15.

In certain embodiments, the peptide dimers comprise two peptides dimerized via a disulfide linkage between a cysteine residue present in one of the peptides and a cysteine residue present in the second peptide, i.e., an intermolecular disulfide bond between these cysteine residues.

In certain embodiments, the peptide dimers comprise two peptides dimerized by covalent attachment of each peptide to a common linking moeity, i.e., a linker. A variety of linkers suitable for dimerizing two peptides are known in the art and commercially available, including. e.g., diethylene glycol (DIG), iminodiacetic acid (IDA), β-Ala-IDA, PEG13, and PEG25, In particular embodiments, peptide dimers include any of the linking moieties shown below or have any of the structures shown below. In particular embodiments, peptide dimers are dimerized via both a linking moiety and a disulphide bond between a cysteine residue in one peptide and a cysteine residue in the other peptide of the dimer.

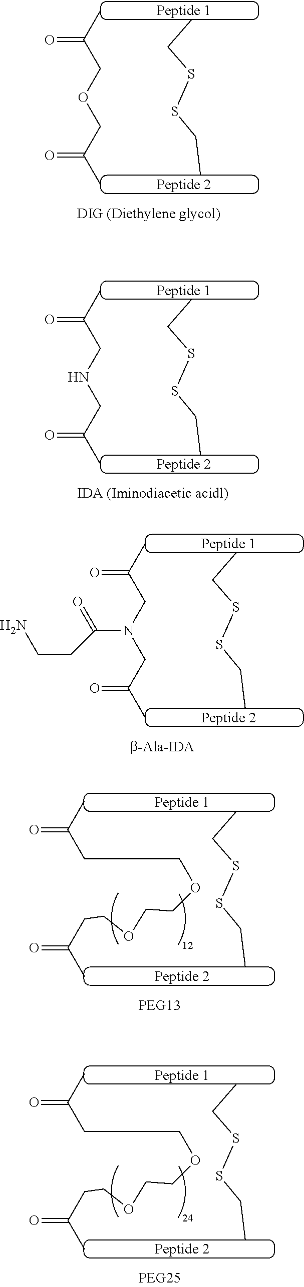

In certain embodiments, the linking moiety comprises the formula: —NH—R₂₀—NH—, wherein R₂₀ is a lower (C₁₋₂₀)alkyl. In certain embodiments, the linking moiety comprises the formula: —CO—(CH₂)n-(X—(CH₂)m)o-X—(CH₂)pCO-, wherein n is 1-3, m is 1-3, p is 1-3, o is 0-24, and X is O or NH. In one embodiment, n, m and p are each 2, o is 1-25, X is O.

In certain embodiments, the linking moiety comprises the formula: —NH—(CH₂)α-[0-(CH₂)β]γO_{δ}—(CH₂)Y—, wherein α, β and ε are each integers whose values are independently selected from 1 to 6, δ is 0 or 1, γ is an integer selected from 0 to 10, and γ is selected from NH or CO, provided that β is 2 when γ is greater than 1.

In various embodiments, the linker is attached to the N-terminal amino acid of one or both peptides of the dimer, the linker is attached to the C-terminal amino acid of one or both peptides of the dimer, or the linker is attached to an internal amino acid of one or both peptides of the dimer. In one embodiment, the linker is attached to lysine residues in each of the peptides of the dimer. In particular embodiments, the linker is not attached to the N-terminal amino acid of one or both peptides of the dimer.

In particular embodiments, one or both peptides present in a dimer comprise an amino acid residue that is conjugated (i.e., covalently attached) to a lipophilic substituent, including any of those described herein. In certain embodiments, one or both peptides present in a dimer comprise an amino acid residue that is conjugated to a polymeric moiety, including any of those described herein. In certain embodiments, one or both of the peptides present in the peptide dimers is conjugated to an acidic compound, e.g., isovaleric acid, isobutyric acid, valeric acid, or the like.

In particular embodiments, a linking moiety present in a dimer is conjugated (i.e., covalently attached) to a lipophilic substituent, including any of those described herein. In certain embodiments, a linking moiety present in a dimer is conjugated to a polymeric moiety, including any of those described herein. In certain embodiments, a linking moiety present in a peptide dimer is conjugated to an acidic compound, e.g., isovaleric acid, isobutyric acid, valeric acid, or the like.

### Pharmaceutical Compositions

It is to be understood that the inclusion of a peptide analogue or a dimer thereof of the invention (i.e., one or more peptide analogues of the invention or one or more peptide dimers of the present invention) in a pharmaceutical composition also encompasses inclusion of a pharmaceutically acceptable salt or solvate of a peptide analogue or a peptide dimer of the invention.

The invention also provides a pharmaceutical composition comprising a peptide analogue, or a pharmaceutically acceptable salt or solvate thereof, according to the invention. In particular embodiments, the invention provides a pharmaceutical composition comprising a peptide dimer, or a pharmaceutically acceptable salt or solvate thereof, according to the invention. In particular embodiments, the pharmaceutical compositions further comprise one or more pharmaceutically acceptable carrier, excipient, or vehicle

The invention also provides a pharmaceutical composition comprising a peptide analogue, or a pharmaceutically acceptable salt or solvate thereof, for treating a variety of conditions, diseases, or disorders as disclosed herein elsewhere (see, e.g., therapeutic uses, supra). In particular embodiments, the invention provides a pharmaceutical composition comprising a peptide dimer, or a pharmaceutically acceptable salt or solvate thereof, for treating a variety of conditions, diseases, or disorders as disclosed herein elsewhere (see, e.g., therapeutic uses, supra).

The peptide analogues, including the peptide dimers, of the present invention may be formulated as pharmaceutical compositions which are suited for administration with or without storage, and which typically comprise a therapeutically effective amount of at least one peptide analogue of the invention, together with a pharmaceutically acceptable carrier, excipient or vehicle.

The term “pharmaceutically acceptable carrier” includes any of the standard pharmaceutical carriers. Pharmaceutically acceptable carriers for therapeutic use are well known in the pharmaceutical art and are described, for example, in “Remington's Pharmaceutical Sciences”, 17th edition, Alfonso R. Gennaro (Ed.), Mark Publishing Company, Easton, PA, USA, 1985. For example, sterile saline and phosphate-buffered saline at slightly acidic or physiological pH may be used. Suitable pH-buffering agents may, e.g., be phosphate, citrate, acetate, tris(hydroxymethyl) aminomethane (TRIS), N-tris(hydroxymethyl)methyl-3˜ aminopropanesulfonic acid (TAPS), ammonium bicarbonate, diethanolamine, histidine, arginine, lysine or acetate (e.g. as sodium acetate), or mixtures thereof. The term further encompasses any carrier agents listed in the US Pharmacopeia for use in animals, including humans.

A pharmaceutical composition of the invention may be in unit dosage form. In such form, the composition is divided into unit doses containing appropriate quantities of the active component or components. The unit dosage form may be presented as a packaged preparation, the package containing discrete quantities of the preparation, for example, packaged tablets, capsules or powders in vials or ampoules. The unit dosage form may also be, e.g., a capsule, cachet or tablet in itself, or it may be an appropriate number of any of these packaged forms. A unit dosage form may also be provided in single-dose injectable form, for example in the form of a pen device containing a liquid-phase (typically aqueous) composition. Compositions may be formulated for any suitable route and means of administration. Pharmaceutically acceptable carriers or diluents include those used in formulations suitable for e.g. oral, intravitreal, rectal, vaginal, nasal, topical, enteral or parenteral (including subcutaneous (SC), intramuscular (IM), intravenous (IV), intradermal and transdermal) administration or administration by inhalation. The formulations may conveniently be presented in unit dosage form and may be prepared by any of the methods well known in the art of pharmaceutical formulation.

Subcutaneous or transdermal modes of administration may be particularly suitable for the peptide analogues of the invention.

Further embodiments of the invention relate to devices, dosage forms and packages used to deliver the pharmaceutical formulations of the present invention. Thus, at least one peptide analogue or specified portion or variant in either the stable or preserved formulations or solutions described herein, can be administered to a patient in accordance with the present invention via a variety of delivery methods, including SC or IM injection; transdermal, pulmonary, transmucosal, implant, osmotic pump, cartridge, micro pump, or other means appreciated by the skilled artisan as well-known in the art.

Still further embodiments of the invention relate to oral formulations and oral administration. Formulations for oral administration may rely on the co-administration of adjuvants (e.g. resorcinols and/or nonionic surfactants such as polyoxyethylene oleyl ether and n-hexadecylpolyethylene ether) to artificially increase the permeability of the intestinal walls, and/or the co-administration of enzymatic inhibitors (e.g. pancreatic trypsin inhibitors, diisopropylfluorophosphate (DFF) or trasylol) to inhibit enzymatic degradation. The active constituent compound of a solid-type dosage form for oral administration can be mixed with at least one additive, such as sucrose, lactose, cellulose, mannitol, trehalose, raffinose, maltitol, dextran, starches, agar, alginates, chitins, chitosans, pectins, gum tragacanth, gum arabic, gelatin, collagen, casein, albumin, synthetic or semisynthetic polymer, or glyceride. These dosage forms can also contain other type(s) of additives, e.g., inactive diluting agent, lubricant such as magnesium stearate, paraben, preserving agent such as sorbic acid, ascorbic acid, alpha-tocopherol, antioxidants such as cysteine, disintegrators, binders, thickeners, buffering agents, pH adjusting agents, sweetening agents, flavoring agents or perfuming agents.

### Dosages

A typical dosage of a peptide analogue, e.g., a peptide or a dimer of the invention, as employed in the context of the present invention may be in the range from about 0.0001 to about 100 mg/kg body weight per day, such as from about 0.0005 to about 50 mg/kg body weight per day, such as from about 0.001 to about 10 mg/kg body weight per day, e.g. from about 0.01 to about 1 mg/kg body weight per day, administered in one or more doses, such as from one to three doses. As already indicated to some extent above, the exact dosage employed will depend, inter alia, on: the nature and severity of the disease or disorder to be treated; the sex, age, body weight and general condition of the subject to be treated; possible other, concomitant, disease or disorder that is undergoing or is to undergo treatment; as well as other factors that will be known to a medical practitioner of skill in the art.

A peptide analogue, e.g., a peptide or a dimer, of the invention may be administered continuously (e.g. by intravenous administration or another continuous drug administration method), or may be administered to a subject at intervals, typically at regular time intervals, depending on the desired dosage and the pharmaceutical composition selected by the skilled practitioner for the particular subject. Regular administration dosing intervals include, e.g., once daily, twice daily, once every two, three, four, five or six days, once or twice weekly, once or twice monthly, and the like.

Such regular peptide analogue, peptide, or dimer administration regimens of the invention may, in certain circumstances such as, e.g., during chronic long-term administration, be advantageously interrupted for a period of time so that the medicated subject reduces the level of or stops taking the medication, often referred to as taking a “drug holiday.” Drug holidays are useful for, e.g., maintaining or regaining sensitivity to a drug especially during long-term chronic treatment, or to reduce unwanted side-effects of long-term chronic treatment of the subject with the drug. The timing of a drug holiday depends on the timing of the regular dosing regimen and the purpose for taking the drug holiday (e.g., to regain drug sensitivity and/or to reduce unwanted side effects of continuous, long-term administration). In some embodiments, the drug holiday may be a reduction in the dosage of the drug (e.g. to below the therapeutically effective amount for a certain interval of time). In other embodiments, administration of the drug is stopped for a certain interval of time before administration is started again using the same or a different dosing regimen (e.g. at a lower or higher dose and/or frequency of administration). A drug holiday of the invention may thus be selected from a wide range of time-periods and dosage regimens. An exemplary drug holiday is two or more days, one or more weeks, or one or more months, up to about 24 months of drug holiday. So, for example, a regular daily dosing regimen with a peptide, a peptide analogue, or a dimer of the invention may, for example, be interrupted by a drug holiday of a week, or two weeks, or four weeks, after which time the preceding, regular dosage regimen (e.g. a daily or a weekly dosing regimen) is resumed. A variety of other drug holiday regimens are envisioned to be useful for administering the peptides, the dimers, and the peptide analogues of the invention.

Thus, the peptide analogue, peptide, or dimer may be delivered via an administration regime which comprises two or more administration phases separated by respective drug holiday phases.

During each administration phase, the peptide analogue, peptide, or dimer is administered to the recipient subject in a therapeutically effective amount according to a pre-determined administration pattern. The administration pattern may comprise continuous administration of the drug to the recipient subject over the duration of the administration phase. Alternatively, the administration pattern may comprise administration of a plurality of doses of the peptide analogue to the recipient subject, wherein said doses are spaced by dosing intervals.

A dosing pattern may comprise at least two doses per administration phase, at least five doses per administration phase, at least 10 doses per administration phase, at least 20 doses per administration phase, at least 30 doses per administration phase, or more.

Said dosing intervals may be regular dosing intervals, which may be as set out above, including once daily, twice daily, once every two, three, four, five or six days, once or twice weekly, once or twice monthly, or a regular and even less frequent dosing interval, depending on the particular dosage formulation, bioavailability, and pharmacokinetic profile of the peptide analogue the peptide, or the peptide dimer of the present invention.

An administration phase may have a duration of at least two days, at least a week, at least 2 weeks, at least 4 weeks, at least a month, at least 2 months, at least 3 months, at least 6 months, or more.

Where an administration pattern comprises a plurality of doses, the duration of the following drug holiday phase is longer than the dosing interval used in that administration pattern. Where the dosing interval is irregular, the duration of the drug holiday phase may be greater than the mean interval between doses over the course of the administration phase. Alternatively the duration of the drug holiday may be longer than the longest interval between consecutive doses during the administration phase.

The duration of the drug holiday phase may be at least twice that of the relevant dosing interval (or mean thereof), at least 3 times, at least 4 times, at least 5 times, at least 10 times, or at least 20 times that of the relevant dosing interval or mean thereof.

Within these constraints, a drug holiday phase may have a duration of at least two days, at least a week, at least 2 weeks, at least 4 weeks, at least a month, at least 2 months, at least 3 months, at least 6 months, or more, depending on the administration pattern during the previous administration phase.

An administration regime comprises at least 2 administration phases. Consecutive administration phases are separated by respective drug holiday phases. Thus the administration regime may comprise at least 3, at least 4, at least 5, at least 10, at least 15, at least 20, at least 25, or at least 30 administration phases, or more, each separated by respective drug holiday phases.

Consecutive administration phases may utilise the same administration pattern, although this may not always be desirable or necessary. However, if other drugs or active agents are administered in combination with a peptide analogue, a peptide or a peptide dimer of the invention, then typically the same combination of drugs or active agents is given in consecutive administration phases. In certain embodiments, the recipient subject is human.

### Devices and Kits

In some embodiments, the invention relates to a device comprising one or more peptides, peptide analogues, peptide dimersor pharmaceutically acceptable salts or solvates thereof of the invention, for delivery of the compound of the present invention to a subject. Thus, one or more peptide analogues, peptides, dimers, or pharmaceutically acceptable salts or solvates thereof can be administered to a patient in accordance with the present invention via a variety of delivery methods including intravenous, subcutaneous, intramuscular, or intraperitoneal injection; oral administration, transdermally, by pulmonary or transmucosal administration, by implant of osmotic pump, by cartridge or micro pump, or by other means appreciated by the skilled artisan, as well-known in the art.

In some embodiments, the invention relates to a kit comprising one or more peptide analogues or pharmaceutically acceptable salts or solvates thereof of the invention. In some embodiments, the invention relates to a kit comprising one or more peptide dimer of the present invention, or pharmaceutically acceptable salts or solvates thereof. In other embodiments, the kit comprises one or more pharmaceutical compositions comprising one or more peptide analogues or pharmaceutically acceptable salts or solvates thereof. In certain embodiments, the kit further comprises packaging or instructions for use. In other embodiments, the kit comprises one or more pharmaceutical compositions comprising one or more peptide dimer of the present invention, or pharmaceutically acceptable salts or solvates thereof. In certain embodiments, the kit further comprises packaging or instructions for use.

### Combination Therapy

As noted above, it will be understood that reference in the following to a peptide analogue of the invention (e.g., the compounds listed in any one of Tables 5-15, for example compounds 1, 2, 3, 4, 5, 6, 7, 8, 9, 10, 11, 12, 13, 14, 15, 16, 17, 18, 19. 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, $1, 52, 53. 54, 55, 56, 57, 58, 59, 60, 65, 66, 67, 68. 69, 70, 71, 73, 74, 75, 76, 78, 79, 80, 81, 82, 83, 84, 85, 86, 87, 88, 89, 90, 91, 92, 93, 94, 95, 96, 97, 98, 99, 100, 101, 102, 103, 105, 106, 107, 108, 109, 110, 111, 112, 113, 114, 115, 117, 118, 119, 120, 121, 122, 123, 124, 125, 126, 127, 128, 129, 130, 131, 132, 133, 134, 135, 136, 137, 139, 140, 141, 142, 143, 144, 145, 146, 147, 148, 149, 1SO, 151, 152, 153, 154, 155, 156, 1$7, 158, 159, 160, 161, 162, 163, 164, 165, 166, 167, 168, 169, 170, 171, 172, 173, 176, 177, 178, 179, 180, 181. 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219. 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 293, 297, 298, 299, 300, 301, 302, 303, 304, 305, 306, 307, 308, 309, 310, 355, 356, 357, 358, 359, 360, 361 or dimers thereof, e.g., any one of the peptide dimers disclosed in Tables 12-15, for example compounds 311-353 also extends to & pharmaceutically acceptable salt or solvate thereof, as well as to a composition comprising more than one different peptide, peptide analogue, or peptide dimer of the invention.

In certain embodiments, a peptide analogue or a peptide dimer of the invention may have some benefit if administered in combination with an iron chelator, such as Deferoxamine and Deferasirox (Exjade™)

### EXAMPLES

The following examples demonstrate certain specific embodiments of the present invention. The following examples were carried out using standard techniques that are well known and routine to those of skill in the art, except where otherwise described in detail. It is to be understood that these examples are for illustrative purposes only and do not purport to be wholly definitive as to conditions or scope of the invention. As such, they should not be construed in any way as limiting the scope of the present invention.

### Abbreviations

DCM: dichloromethane
DMF: N,N-dimethylformamide
NMP: N-methylpyrolidone
HBTU: O-(Benzotriazol-1-yl)-N,N,N′,N′-tetramethyluronium hexafluorophosphate
HATU: 2-(7-aza-IH-benzotriazole-1-yl)-1,1,3,3 tetramethyluronium hexafluorophosphate
DCC: Dicyclohexylcarbodiimide
NHS: N-hydoxysuccinimide
DIPEA: diisopropylethylamine
EtOH: ethanol
Et2O: diethyl ether
Hy: hydrogen
TFA: trifluoroacetic acid
TIS: triisopropylsilane
ACN: acetonitrile
HPLC: high performance liquid chromatography
ESI-MS: electron spray ionization mass spectrometry
PBS: phosphate-buffered saline
Boc: t-butoxy carbonyl
Fmoc: Fluorenylmethyloxycarbonyl
Acm: acetamidomethyl
IVA: Isovaleric acid (or Isovaleryl)
K( ): In the peptide sequences provided herein, wherein a compound or chemical group is presented in parentheses directly after a Lysine residue, it is to be understood that the compound or chemical group in the parentheses is a side chain conjugated to the Lysine residue. So, e.g., but not to be limited in any way, K (PEGB) indicates that a PEG8 moiety is conjugated to a side chain of this Lysine. For a few non-limiting examples of such a conjugated Lysines, please see, e.g., compounds 54 and 90.
Palm: Indicates conjugation of a palmitic acid (palmitoyl).

As used herein “C( )” refers to a cysteine residue involved in a particular disulfide bridge. For example, in Hepcidin, there are four disulfide bridges: the first between the two C(1) residues; the second between the two C(2) residues; the third between the two C(3) residues; and the fourth between the two C(4) residues. Accordingly, in some embodiments, the sequence for Hepcidin is written as follows:
Hy-DTHEPIC(1)IFC(2)C(3)GC(2)C(4)HRSKC(3)GMC(4)C(1)KT-OH(SEQ ID NO:335); and the sequence for other peptides may also optionally be written in the same manner.

The following examples are provided to illustrate certain embodiments of the invention and are not intended to limit the scope of the invention.

### Example 1

### Synthesis of Compounds

Unless otherwise specified, reagents and solvents employed in the following were available commercially in standard laboratory reagent or analytical grade, and were used without further purification.

### Procedure for Solid-Phase Synthesis of Peptides

Illustrative compounds of the invention (e.g., Compound No. 2) were chemically synthesized using optimized 9-fluorenylmethoxy carbonyl (Fmoc) solid phase peptide synthesis protocols. For C-terminal amides, rink-amide resin was used, although wang and trityl resins were also used to produce C-terminal acids. The side chain protecting groups were as follows: Glu, Thr and Tyr: O-tButyl; Trp and Lys: t-Boc (t-butyloxycarbonyl); Arg: N-gamma-2,2,4,6,7-pentamethyldihydrobenzofuran-5-sulfonyl; His, Gin, Asn, Cys: Trityl. For selective disulfide bridge formation, Acm (acetamidomethyl) was also used as a Cys protecting group. For coupling, a four to ten-fold excess of a solution containing Fmoc amino acid, HBTU and DIPEA (1:1:1.1) in DMF was added to swelled resin [HBTU: O)-(Benzotriazol-1-yl)-N,N,N′,N′-tetramethyluronium hexafluorophosphate; DIPEA: diisopropylethylamine; DMF: dimethylformamide]. HATU (O-(7-azabenzotriazol-1-yl)-1,1,3,3,-tetramethyluronium hexafluorophosphate) was used instead of HBTU to improve coupling efficiency in difficult regions. Fmoc protecting group removal was achieved by treatment with a DMF, piperidine (2:1) solution.

### Procedure for Cleavage of Peptides Off Resin

Side chain deprotection and cleavage of the peptides of the invention (e.g., Compound No. 2) was achieved by stirring dry resin in a solution containing trifluoroacetic acid, water, ethanedithiol and tri-isopropylsilane (90:5:2.5:2.5) for 2 to 4 hours. Following TFA removal, peptide was precipitated using ice-cold diethyl ether. The solution was centrifuged and the ether was decanted, followed by a second diethyl ether wash. The peptide was dissolved in an acetonitrile, water solution (1:1) containing 0.1% TFA (trifluoroacetic acid) and the resulting solution was filtered. The linear peptide quality was assessed using electrospray ionisation mass spectrometry (ESI-MS).

### Procedure for Purification of Peptides

Purification of the peptides of the invention (e.g., Compound No. 2) was achieved using reverse-phase high performance liquid chromatography (RP-HPLC). Analysis was performed using a C18 column (3 μm, 50×2 mm) with a flow rate of 1 ml./min. Purification of the linear peptides was achieved using preparative RP-HPLC with a C18 column (5 μm, 250×21.2 mm) with a flow rate of 20 mL/min. Separation was achieved using linear gradients of buffer Bin A (Buffer A: Aqueous 0.05% TFA; Buffer B: 0.043% TFA, 90% acetonitrile in water).

### Procedure for Oxidation of Peptides

Method A (Single disulfide oxidation). Oxidation of the unprotected peptides of the invention (e.g., Compound No. 2) was achieved by adding drop-wise iodine in MeOH (1 mg per 1 mL) to the peptide in a solution (ACN: ILO, 7:3, 0.5% TFA). After stirring for 2 min, ascorbic acid portion wise was added until the solution was clear and the sample was immediately loaded onto the HPLC for purification.

Method B (Selective oxidation of two disulfides). When more than one disulfide was present (e.g., Compound 30), selective oxidation was often performed. Oxidation of the free cysteines was achieved at pH 7.6 NH₄CO₃ solution at 1 mg/10 mL of peptide. After 24 h stirring and prior to purification the solution was acidified to pH 3 with TFA followed by lyophilization. The resulting single oxidized peptides (with ACM protected cysteines) were then oxidized/selective deprotection using iodine solution. The peptide (1 mg per 2 mL) was dissolved in MeOH/H: 0, 80:20 iodine dissolved in the reaction solvent was added to the reaction (final concentration: 5 mg/mL) at room temperature. The solution was stirred for 7 minutes before ascorbic acid was added portion wise until the solution is clear. The solution was then loaded directly onto the HPLC.

Method C(Native oxidation). When more than one disulfide was present and when not performing selective oxidations, native oxidation was performed (e.g., this method was used for Compound 19). Native oxidation was achieved with 100 mM NH4CO3 (pH7.4) solution in the presence of oxidized and reduced glutathione (peptide/GSH/GSSG, 1:100:10 molar ratio) of (peptide: GSSG: GSH, 1:10, 100). After 24 h stirring and prior to RP-HPLC purification the solution was acidified to pH 3 with TFA followed by lyophilization.

Procedure of Cysteine oxidation to produce dimers. Oxidation of the unprotected peptides of the invention (e.g., Compound No. 1) was achieved by adding drop-wise iodine in MeOH (1 mg per 1 ml) to the peptide in a solution (ACN. H2O, 7:3, 0.5% TFA). After stirring for 2 min, ascorbic acid portion wise was added until the solution was clear and the sample was immediately loaded onto the HPLC for purification.

Procedure for Dimerization. Glyxoylic acid, IDA, or Fmoc-B-Ala-IDA was pre-activated as the N-hydoxysuccinimide ester by treating the acid (1 equiv) with 2.2 eq of both N-hydoxysuccinimide (NHS) and dicyclohexyl carbodiimide (DCC) in NMP (N-methyl pyrolidonej at a 0.1 M final concentration. For the PEG13 and PEG25 linkers, these chemical entities were purchased pre-formed as the activated succinimide ester. The activated ester ˜0.4 eq was added slowly to the peptide in NMP (1 mg/mL) portionwise. The solution was left stirring for 10 min before 2-3 additional aliquots of the linker ˜0.05 eq were slowly added. The solution was left stirring for a further 3 h before the solvent was removed under vaccuo and the residue was purified by reverse phase HPLC. An additional step of stirring the peptide in 20% piperidine in DMF (2×10 min) before an additional reverse phase HPLC purification was performed.

One of skill in the art will appreciate that standard methods of peptide synthesis may be used to generate the compounds of the invention.

### Example 2

### Activity Assays Methodology

The designed peptides were tested in vitro for induction of degradation of the human ferroportin protein.

The cDNA encoding the human ferroportin (SLC40A1) was cloned from a cDNA clone from Origene (NM_014585). The DNA encoding the ferroportin was amplified by PCR using primers also encoding terminal restriction sites for subcloning, but without the termination codon. The ferroportin receptor was subcloned into a mammalian GFP expression vector containing a neomycin (G418) resistance marker in such that the reading frame of the ferroportin was fused in frame with the GFP protein. The fidelity of the DNA encoding the protein was confirmed by DNA sequencing. HEK293 cells were used for transfection of the ferroportin-GFP receptor expression plasmid. The cells were grown according to standard protocol in growth medium and transfected with the plasmids using Lipofectamine (manufacturer's protocol, Invitrogen). The cells stably expressing ferroportin-GFP were selected using G418 in the growth medium (in that only cells that have taken up and incorporated the cDNA expression plasmid survive) and sorted several times on a Cytomation MoFlo™ cell sorter to obtain the GFP-positive cells (488 nm/530 nm). The cells were propagated and frozen in aliquots.

To determine compound activity on the human ferroportin, the cells were incubated in 96 well plates in standard media, without phenol red. Compound was added to desired final concentration for at least 18 hours in the incubator. Following incubation, the remaining GFP-fluorescence was determined either by whole cell GFP fluorescence (Envision plate reader, 485/$35 filter pair), or by Beckman Coulter Quanta™ flow cytometer (express as Geometric mean of fluorescence intensity at 485 nm/525 nm). Compound was added to desired final concentration for at least 18 hours but no more than 24 hours in the incubator.

Reference compounds included native Hepcidin, Mini-Hepcidin, and RI-Mini-Hepcidin, which is an analog of mini-hepcidin. The “RI” in RI-Mini-Hepcidin refers to Retro Inverse. A retro inverse peptide is a peptide with a reversed sequence in all D amino acids. An example is that Hy-Glu-Thr-His-NH2 becomes Hy-DHis-DThr-Dglu-NH2. The EC50 of these reference compounds for ferroportin degradation was determined according to the activity assay described above. These peptides served as control standards for many of the subsequence studies.

TABLE 4
Reference compounds

| | | SEQ | EC50 |
|---|---|---|---|
| Name | Sequence | ID No. | (nM) |
| Hepcidin | Hy-DTHFPIC(1)IFC(2)C(3) | 335 | 169 |
| | GC(2)C(4)HRSKC(3)GMC(4) | | |
| | C(1)KT-OH | | |
| Mini- | Hy-DTHFPICIF-NH₂ | 336 | 712 |
| Hepcidin | | | |
| 1-9 | | | |
| RI-Mini | Hy-DPhe-DIle- | 337 | >10 μM |
| Hepcidin | DCys-DIle-DPro-DPhe- | | |
| | DHis-DThr-DAsp-NH₂ | | |

To determine whether a given peptide modifies the internalization and degradation of endogenous ferroportin, the protein levels and cellular distribution of ferroportin in hepatocytes and macrophages treated with the peptide may be assayed using Western blotting, immunohistochemistry and ferroportin antibodies known in the art.

### Example 3

### Cysteine Replacement Scan of Mini-Hepcidin

Previous studies indicate that the N-terminal segment of Hep25 is important for its hepcidin activity and is likely to form the interface with ferroportin. Furthermore, it was thought that Cys in the 7^{th} position is critical for activity. Disulfide bonds can act by structural, catalytic or by functional means. It is postulated that Hepcidin binds to Ferroportin through a disulphide linkage which subsequently internalizes the receptor. A closer inspection of hepcidin reveled that there are 4 disulfides present and that, any one of these cysteine might be responsible for binding to ferroportin. As such, the free thiol of ferroportin possesses a “functional, allosteric bond” equivalent. In order to more thoroughly understand the structure activity relationship with respect to the position of the cysteines within Hepcidin, we performed a cysteine scan up to the 15^{th} residue of a mini-hepcidin peptide and we analyzed the peptides for their ability to exhibit hepcidin activity. Peptides were synthesized using the methods described in Example I, and their potency for ferroportin degradation was tested as described in Example 2. Results of this study are shown in Table 5, with potency indicated by EC50 values.

TABLE 5
Cysteine replacement scan
of Mini-Hepcidin derivatives

| Compound | SEQ | | EC50 (nM) |
|---|---|---|---|
| Number | ID No. | Sequence | (n > 3) |
| 269 | 292 | DTHFPIAIFAAGICI-NH₂ | Not active |
| 270 | 293 | DTHFPIAIFAAICI-NH₂ | Not active |
| 271 | 294 | DTHFPIAIFAICI-NH₂ | Not active |
| 272 | 295 | DTHFPIAIFICI-NH₂ | Not active |
| 273 | 296 | DTHFPIAIICI-NH₂ | Not active |
| 274 | 297 | DTHFPIAICI-NH₂ | Not active |
| 275 | 298 | DTHFPIICI-NH₂ | Not active |
| Mini- | 336 | Hy-DTHFPICIF-NH₂ | 712 nM |
| Hepcidin | | | |
| 1-9 | | | |
| 1 | 28 | DTHFPCIIF-NH₂ | 133 nM |
| 276 | 299 | DTHICIAIF-NH₂ | Not active |
| 277 | 300 | DTHCPIAIF-NH₂ | Not active |

Inactive = Not active at 30 μM and/or lowest dose

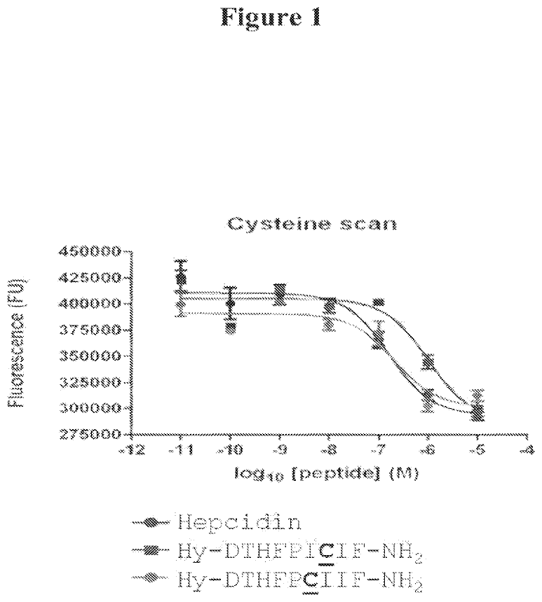

Altering the position of the cysteine ablated activity for most of the peptides that were tested; however these data surprisingly demonstrated that Compound 1 is active despite having a Cysteine at the 6^{th} position. FIG. 1 shows a comparison of the dose response curves for Compound 1, as compared to Hepcidin, and the Mini-Hepcidin control. These data clearly demonstrate that Compound 1 has similar in-vitro potency as Hepcidin.

### Example 4

### Ala Scans of Compound 1 Identified in Cysteine Scan

To validate the results from Example 3, an Ala scan was performed on Compound 1. Peptides were synthesized as described in Example 1, and they were tested for activity as described in Example 2. The results of this study are shown in Table 6. By comparing this result with known structure activity relationships with hepcidin and other mini-hepcidin analogs, we have increased potency. Moreover, these data clearly demonstrate the importance of several residues for activity. Conversely, these date also identify a number of residues that can be modified without ablating activity.

TABLE 6
Alanine scan of Compound 1

| Compound | SEQ | | EC50 (nM) |
|---|---|---|---|
| Number | ID No. | Sequence | (n > 3) |
| 1 | 28 | DTHFPCIIF-NH₂ | 133 nM |
| 278 | 301 | DTHFPCIIA-NH₂ | >1 μM |
| 51 | 78 | DTHFPCIAF-NH₂ | 382 nM |
| 279 | 302 | DTHFPCAIF-NH₂ | >1 μm |
| 280 | 303 | DTHFACIIF-NH₂ | >1 μM |
| 282 | 305 | DTHAPCIIF-NH₂ | Not active |
| 283 | 306 | DTAFPCIIF-NH₂ | 739 nM |
| 52 | 79 | DAHFPCIIF-NH₂ | 388 nM |
| 284 | 307 | ATHFPCIIF-NH₂ | >1 μM |
| 281 | 304 | DTHF-[(D)-AlA]- | Not active |
| | | CIIF-NH₂ | |

### Example 5

### Analysis of Peptitde Activities In Vitro

Based in part on the structure activity relationships (SAR) determined from the results of the experiments described in Examples 3 and 4, a variety of Hepcidin-like peptides of the present invention were synthesized using the method described in Example 1, and in vitro activity was tested as described in Example 2. Reference compounds (shown in Table 4) included native Hepcidin, Mini-Hepcidin, and R1-Mini-Hepcidin. EC50 values of the peptides are shown in summary Table 7.

TABLE 7
In vitro activity of Hepcidin analog peptides

| | SEQ | | Potency |
|---|---|---|---|
| No. | ID No. | Sequence | EC₅₀ (nM) |
| 1 | 28 | Hy-DTHFPCIIF-NH₂ | 133 |
| 2 | 29 | Isovaleric acid-DTHFPICIFGPRSKGWVC-NH₂ | 5 |
| 3 | 30 | Isovaleric acid-DTHFPCIIFGPRSRGWVCK-NH₂ | 15 |
| 4 | 31 | Isovaleric acid-DTHFPCIIFGPRSKGWVC-NH₂ | 19 |
| 5 | 32 | [Ida]-TH-[Dpa]-[bhPro]-ICIFGPRSKGWVCM-NH₂ | 17 |
| 6 | 33 | Isovaleric acid-DTHFPCIFFGPRSKGWVCK-NH₂ | 23 |
| 7 | 34 | Isovaleric acid-DTHFPCIIFGPRSKGWTCK-NH₂ | 24 |
| 8 | 35 | [Ida]-TH-[Dpa]-[bh-Pro]-CIIFGPRSRGWVCK-NH₂ | 29 |
| 9 | 36 | Isovaleric acid-DTHFPCIKFGPRSKGWVCK-NH₂ | 32 |
| 10 | 37 | Isovaleric acid-DTHFPCIQFGPRSKGWVCK-NH₂ | 35 |
| 11 | 38 | Isovaleric acid-DTHFPCIIFGPRSKGWVCK-NH₂ | 9 |
| 12 | 39 | Hy-DTHFPIC₁IFVC₂GHRSIC₂YRRC₁R-NH₂ | 77 |
| 13 | 40 | Isobutyric acid-DTHFPIC₁IFVC₂HRSKGC₂YRRC₁R-NH₂ | 63 |
| 14 | 41 | Hy-DTHFPIC₁IFVC₂HRSKGC₂YRAC₁-NH₂ | 69 |
| 15 | 42 | Isovaleric acid-DTHFPCIEFGPRSKGWVCK-NH₂ | 79 |
| 16 | 43 | Hy-DTHFPICIFGPRAKGWVCM-NH₂ | 88 |
| 17 | 44 | Isobutyric acid-DTHFPIC₁IFVC₂HRSKGC₂YRRC₁R-NH₂ | 93 |
| 18 | 45 | Hy-DTHFPICIFGPRSKGWVCM-NH₂ | 125 |
| 19 | 46 | Hy-DTHFPIC₁IFVC₂HRSKGC₂YRRC₁R-NH₂ | 140 |
| 20 | 47 | Hy-DTHFPICIFGPRSRGWVCK-NH₂ | 101 |
| 21 | 48 | Hy-DTHFPCIIFGPRSKGWVCM-NH₂ | 46 |
| 22 | 49 | Hy-DTHFPICIFAPRSKGWVCM-NH₂ | 9430 |
| 23 | 50 | Hy-DTHFPICIFGPRSKGWVCM-OH | 131 |
| 24 | 51 | Hy-DTHFPCIQF-NH₂ | 138 |
| 25 | 52 | Hy-DTHFPIC₁IFVC₂GHRSKGC₂YRRC₁R-NH₂ | 144 |
| 26 | 53 | Hy-DTHFAICIFGPRSKGWVCM-NH₂ | 147 |
| 27 | 54 | Hy-DTHFPICIFGPHRSKGWVCM-NH₂ | 149 |
| 28 | 55 | Hy-DTHFPICIFGPRAKGWVCM-NH₂ | 88 |
| 29 | 56 | Hy-DTHFPACIFGPRSKGWVCM-NH₂ | 157 |
| 30 | 57 | Hy-DTHFPC₁IIFVC₂HRPKGC₂YRRVC₁R-NH₂ | 173 |
| 31 | 58 | Hy-DTHFPICIFGPRSKAWVCM-NH₂ | 175 |
| 32 | 59 | Hy-DTHFPIC₁IFVC₂GHRGKGC₂YRRC₁R-NH₂ | 182 |
| 33 | 60 | Hy-ATHFPICIFGPRSKGWVCM-NH₂ | 184 |
| 34 | 61 | Hy-DTHFPICIFGPASKGWVCM-NH₂ | 206 |
| 35 | 62 | Hy-DTHFPIC₁IFVC₂HRSKGC₂YARC₁-NH₂ | 214 |
| 36 | 63 | Ac-DTHFPICIFGPRSKGWVCM-NH₂ | 239 |
| 37 | 64 | Hy-DTHFPICIFGPRSAGWVCM-NH₂ | 239 |
| 38 | 65 | Hy-DTHAPICIFGPRSKGWVCM-NH₂ | 254 |
| 39 | 66 | Hy-DTHFPIC₁IFVC₂HRSKGC₂YRRC₁-NH₂ | 256 |
| 40 | 67 | pGlu-THFPIC₁IFVC₂HRSKGC₂YRRC₁R-NH₂ | 260 |
| 41 | 68 | Ac-DTHFPICIFKPRSKGWVCM-NH₂ | 262 |
| 42 | 69 | Hy-DTHFPIC₁IFVC₂GHRSKGC₂YMRC₁KT-NH₂ | 265 |
| 43 | 70 | Hy-DAHFPICIFGPRSKGWVCM-NH₂ | 265 |
| 44 | 71 | Hy-DTHFPIC₁IFVC₂YRGIC₂YRRC₁R-NH₂ | 269 |
| 45 | 72 | Ac-DTHFPICIFGPRSKGWVCM-NH₂ | 272 |
| 46 | 73 | Hy-[bhAsp]-THFPICIFGPRSKGWVC-NH₂ | 274 |
| 47 | 74 | Hy-DTHFPICIFGPRSKGWACM-NH₂ | 313 |
| 48 | 75 | [Ida]-TH-[Dpa]-[bhPro]-RCR-[bhPhe]-GPRSKGWVCM-NH₂ | 331 |
| 49 | 76 | Hy-DTHFPCIRF-NH₂ | 334 |
| 50 | 77 | Isovaleric acid-THFPCIIFGPRSKGWVCM-NH₂ | 345 |
| 51 | 78 | Hy-DTHFPCIAF-NH₂ | 382 |
| 52 | 79 | Hy-DAHFPCIIF-NH₂ | 388 |
| 53 | 80 | Hy-DTHFPIC₁IFVC₂HRPKGC₂YRRC₁P-NH₂ | 393 |
| 54 | 81 | Ac-DTHFPICIFKPRS-K(PEG8)-GWVCM-NH₂ | 479 |
| 55 | 82 | Hy-DTHFPCIIFK-NH₂ | 419 |
| 56 | 83 | Hy-DTHFPCIFF-NH₂ | 441 |
| 57 | 84 | Hy-DTHFPICIFGPRSK-K(PEG8)-WVCM-NH₂ | 462 |
| 58 | 85 | Ac-DTHFPICIFGPRSKKWVCM-NH₂ | 472 |
| 59 | 86 | Hy-DTHFPIC₁IFC₂PWGMC₂C₁K-NH2 | 495 |
| 60 | 87 | Hy-DTAFPICIFGPRSKGWVCM-NH₂ | 498 |
| 65 | 88 | Hy-DTHFPIC₁IFVC₂YRGIC₁YMRC₂KT-NH₂ | 763 |
| 66 | 89 | Hy-DTHFPICIFGPRSKGAVCM-NH₂ | 520 |
| 67 | 90 | Hy-DTHFPICIAGPRSKGWVCM-NH₂ | 2466 |
| 68 | 91 | Hy-DTHFPICAFGPRSKGWVCM-NH₂ | >10 μM |
| 69 | 92 | Hy-DTHFPIAIFGPRSKGWVAM-NH₂ | Inactive |
| 70 | 93 | Hy-DTHFPCRRFGPRSKGWVC-NH₂ | Inactive |
| 71 | 94 | [Ida]-THF-[bh-Pro]-CRR-[bh-Phe]-GPRSKGWVC-NH₂ | N/A |
| 73 | 96 | Hy-DTHFPC₁IIFVC₂HRSKGC₂YWAVC₁-NH₂ | 2640 |
| 74 | 97 | Hy-DTHFP-(D)Cys₁-IIFVC₂HRSKGC₂YWAV-(D)Cys₁-F-NH₂ | 356 |
| 75 | 98 | Hy-DTHFPC₁IIFVC₂HRSKGC₂YWAVC₁FW-NH₂ | Not Tested |
| 76 | 99 | Ac-DTHFPICIF-K(PEG8)-PRSKGWVCM-NH₂ | 610 |
| 78 | 101 | Hy-DTH-[Dpa]-PCIIFGPRSRGWVCK-NH₂ | >1 μM |
| 79 | 102 | Hy-DTHF[bh-Pro]-CIIFGPRSRGWVCK-NH₂ | >1 μM |
| 80 | 103 | Hy-DTHFPCIIFGPRSRGWRCK-NH₂ | >1 μM |
| 81 | 104 | Hy-DTHFPCIRFGPRSRGWVCK-NH₂ | >1 μM |
| 82 | 105 | Hy-DTHFPCIRFGPRSRGWRCK-NH₂ | >1 μM |
| 83 | 106 | Hy-DTHFPCIIFGPRSRGWVCK-NH₂ | >1 μM |
| 84 | 107 | Hy-DTHFPCIIFGPRSRGVCK-NH₂ | >1 μM |
| 85 | 108 | Hy-DTHFPCIYFGPRSKGWVCK-NH₂ | 705 |
| 86 | 109 | Hy-DTHFPCIIFGPRSKGWVCK-NH₂ | >1 μM |
| 87 | 110 | Hy-DTHFPCIIFGPRARGWVCK-NH₂ | >1 μM |
| 88 | 111 | Octanoic acid-DTHFPCIIFGPRSRGWVCK-NH₂ | >1 μM |
| 89 | 112 | Palm-PEG11-DTHFPCIIFGPRSRGWVCK-NH₂ | >1 μM |
| 90 | 113 | Ac-DTHFPICIF-K(2K PEG)-PRSKGWVCK-NH₂ | 107 |
| 91 | 114 | Hy-DTHFPCIIFGPRSKGWKCK-NH₂ | Not Tested |
| 92 | 115 | Hy-DTHFPCIKFGPRSKGWKCK-NH₂ | Not Tested |
| 93 | 116 | Isovaleric acid-DTHFPCLIFGPRSKGWVCK-NH₂ | 19 |
| 94 | 117 | Isovaleric acid-DTHFPCVIFGPRSKGWVCK-NH₂ | 41 |
| 95 | 118 | Isovaleric acid-DTHFPCSIFGPRSKGWVCK-NH₂ | 78 |
| 96 | 119 | Isovaleric acid-DTHFPCQIFGPRSKGWVCK-NH₂ | 157 |
| 97 | 120 | Hy-THFPCIIFGPRSKGWVCK-NH₂ | Inactive |
| 98 | 121 | Isovaleric acid-THFPCIIFGPRSKGWVCK-NH₂ | Inactive |
| 99 | 122 | Hy-HFPCIIFGPRSKGWVCK-NH₂ | Inactive |
| 100 | 123 | Isovaleric acid-HFPCIIFGPRSKGWVCK-NH₂ | Inactive |
| 101 | 124 | Hy-DTHFPCISFGPRSKGWVCK-NH₂ | >1 μM |
| 102 | 125 | Hy-DTHFPCIKFGPRSKGWVCK-NH₂ | >1 μM |
| 103 | 126 | Hy-EDTHFPCIIFGPRSKGWVCK-NH₂ | >1 μM |
| 105 | 128 | Isovaleric acid-DTHFPCIIFEPRSKGWVCK-NH₂ | 10 |
| 106 | 129 | Isovaleric acid-DTHFPCIIFSPRSKGWVCK-NH₂ | 44 |
| 107 | 130 | Isovaleric acid-DTHFSCIIFGPRSKGWVCK-NH₂ | 50 |
| 108 | 131 | Octanoic acid-PEG11-DTHFPCIIFGPRSRGWVCK-NH₂ | >1 μM |
| 109 | 132 | Isobutyric acid-PEG11-DTHFPCIIFGPRSRGWVCK-NH₂ | >1 μM |
| 110 | 133 | [Ida]-THFPCIIFGPRSRGWVCK-NH₂ | >300 nM |
| 111 | 134 | Isovaleric acid-DTHFPCIIFGPKSKGWVCK-NH₂ | 12 |
| 112 | 135 | Isovaleric acid-DTHFPCIKFGPKSKGWVCK-NH₂ | 15 |
| 113 | 136 | Isovaleric acid-DTHFPCIIFGPRSKGWCK-NH₂ | 15 |
| 114 | 137 | Isovaleric acid-DTHFPCIIFGPRSKGVC-NH₂ | 18 |
| 115 | 138 | Isovaleric acid-DTHFPCIIFGPRSKGCK-NH₂ | 21 |
| 117 | 140 | Isovaleric acid-DTHFPC-[Dapa]-IFGPRSKGWDCK-NH₂ | 65 |
| 118 | 141 | Isovaleric acid-DTHFPCI-[Dapa]-FGPRSKGWDCK-NH₂ | 17 |
| 119 | 142 | Isovaleric acid-DTHFPC-[Dapa]-IFGPRSKGWECK-NH₂ | 151 |
| 120 | 143 | Isovaleric acid-DTHFPCI-[Dapa]-FGPRSKGWECK-NH₂ | 15 |
| 121 | 144 | Isovaleric acid-DTHFPCIKFGPRSKGWECK-NH₂ | 14 |
| 122 | 145 | Isovaleric acid-DTHFGCIIFGPRSKGWVCK-NH₂ | 57 |
| 123 | 146 | Hy-DTHFGCIIFGPRSKGWVCK-NH₂ | Inactive |
| 124 | 147 | Isovaleric acid-DTHFRCIIFGPRSKGWVCK-NH₂ | 106 |
| 125 | 148 | Hy-DTHFRCIIFGPRSKGWVCK-NH₂ | Inactive |
| 126 | 149 | Isovaleric acid-DTHF-[Sarc]-CIIFGPRSKGWVCK-NH₂ | 31 |
| 127 | 150 | Hy-DTHF-[Sarc]-CIIFGPRSKGWVCK-NH₂ | Inactive |
| 128 | 151 | Isovaleric acid-DTHF-[β-Ala]-CIIFGPRSKGWVCK-NH₂ | 264 |
| 129 | 152 | Hy-DTHF-[β-Ala]-CIIFGPRSKGWVCK-NH₂ | Inactive |
| 130 | 153 | Isovaleric acid-DTHFKCIIFGPRSKGWVCK-NH₂ | 150 |
| 131 | 154 | Hy-DTHFKCIIFGPRSKGWVCK-NH₂ | Inactive |
| 132 | 155 | Hy-THFPCIIFGPRSKGWVCM-NH₂ | >1 μM |
| 133 | 156 | Hy-HFPCIIFGPRSKGWVCM-NH₂ | >1 μM |
| 134 | 157 | Isovaleric acid-HFPCIIFGPRSKGWVCM-NH₂ | >1 μM |
| 135 | 158 | Hy-DTHFPCISFGPRSKGWVCM-NH₂ | 545 |
| 136 | 159 | Hy-DTHFPCIKFGPRSKGWVCM-NH₂ | 669 |
| 137 | 160 | Hy-EDTHFPCIIFGPRSKGWVCM-NH₂ | 873 |
| 139 | 162 | Hy-DTHFPCIIFEPRSKGWVCM-NH₂ | N/A |
| 140 | 163 | Isovaleric acid-DTHFKCIEFGPRSKGWVCK-NH₂ | >1 μM |
| 141 | 164 | Isovaleric acid-DTHFPCIIFGPRSKGWACK-NH₂ | 11 |
| 142 | 165 | Isovaleric acid-DTHFPCIIFEPRSKGWVCK-NH₂ | 9 |
| 143 | 166 | Isovaleric acid-DTHFPCIIFGPRSKGWVCKKKK-NH₂ | 24 |
| 144 | 167 | Isovaleric acid-DTHFPCIIFEPRSKGWVCKKKK-NH₂ | 15 |
| 145 | 168 | Isovaleric acid-DTHFPCIIFGPRSKGWVCKK-NH₂ | 9 |
| 146 | 169 | Isovaleric acid-DTAFPCIIFGPRSKGWVCK-NH₂ | 24 |
| 147 | 170 | Isovaleric acid-DTKFPCIIFGPRSKGWVCK-NH₂ | 20 |
| 148 | 171 | Isovaleric acid-DTHFPC₁IIFVC₂HRPKGC₂YRRVC₁R-NH₂ | 2.2 |
| 149 | 172 | Isovaleric acid-DTHFPCI-K(PEG8)-FGPRSKGWVCK-NH₂ | 9 |
| 150 | 173 | Isovaleric acid-DTHFPCIKF-K(PEG8)-PRSKGWVCK-NH₂ | 7 |
| 151 | 174 | Isovaleric acid-DTHFPCIKFGP-K(PEG8)-SKGWVCK-NH₂ | 13 |
| 152 | 175 | Isovaleric acid-DTHFPCIKFGPRS-K(PEG8)-GWVCK-NH₂ | 16 |
| 153 | 176 | Isovaleric acid-DTHFPCIKFGPRSKGWVC-K(PEG8)-NH₂ | 18 |
| 154 | 177 | Isovaleric acid-DTHFPCIKFGPRSKGWTCK-NH₂ | 18 |
| 155 | 178 | Isovaleric acid-DTHFPCIEFGPRSKGWTCK-NH₂ | 38 |
| 156 | 179 | Isovaleric acid-DTHFPICIFGPRS-K(Betaine)-GWVC-NH₂ | Not Tested |
| 157 | 180 | Isovaleric acid-DTHFPCIKFGPRS-K(Betaine)-GWVCK-NH₂ | 18 |
| 158 | 181 | Isovaleric acid-DTHFPCI-K(Betaine)-FGPRSKGWVCK-NH₂ | 16 |
| 159 | 182 | Isovaleric acid-DTHFPCIKFGPRSKGWVC-K(Betaine)-NH₂ | 17 |
| 160 | 183 | Ac-DTHFPCIKFGPRSKGWVCK-NH₂ | 464 |
| 161 | 184 | Isovaleric acid-PEG3-DTHFPCIKFGPRSKGWVCK-NH₂ | 666 |
| 162 | 185 | Isobutyric acid-DTHFPCIKFGPRSKGWVCK-NH₂ | 41 |
| 163 | 186 | Valeric acid-DTHFPCIKFGPRSKGWVCK-NH₂ | 64 |
| 164 | 187 | Hy-VDTHFPCIKFGPRSKGWVCK-NH₂ | 146 |
| 165 | 188 | Hy-LDTHFPCIKFGPRSKGWVCK-NH₂ | 107 |
| 166 | 189 | Hexanoic acid-DTHFPCIKFGPRSKGWVCK-NH₂ | 36 |
| 167 | 190 | 5-Methylpentanoic acid-DTHFPCIKFGPRSKGWVCK-NH₂ | 99 |
| 168 | 191 | Cyclohexanoic acid-DTHFPCIKFGPRSKGWVCK-NH₂ | 30 |
| 169 | 192 | Heptanoic acid-DTHFPCIKFGPRSKGWVCK-NH₂ | 91 |
| 170 | 193 | Octanoic acid-DTHFPCIKFGPRSKGWVCK-NH₂ | 183 |
| 171 | 194 | Isovaleric acid-DTHFPCIIFGPRSKGWKCK-NH₂ | 48 |
| 172 | 195 | Isovaleric acid-DTHFPCIIFGPRSKGWECK-NH₂ | 15 |
| 173 | 196 | Isovaleric acid-DTHFPCRRFGPRSKGWVCK-NH₂ | Not Tested |
| 176 | 199 | Isovaleric acid-DTHFPICIFGPRS-K(PEG8)-GWVC-NH₂ | 6 |
| 177 | 200 | Isovaleric acid-DTHFPICIFGPRS-K(PEG4)-GWVC-NH₂ | 6 |
| 178 | 201 | Isovaleric acid-DTHFPCIIFGPRSRGWVC-K(PEG8)-NH₂ | 3 |
| 179 | 202 | Isovaleric acid-DTHFPCIIFGPRSRGWVC-K(PEG4)-NH₂ | 4 |
| 180 | 203 | Isovaleric acid-DTHFPCIIFGPRSRGWVC-K(PEG2)-NH₂ | 9 |
| 181 | 204 | Isovaleric acid-DTHFPCIKFEPRSKGWVCK-NH₂ | 15 |
| 182 | 205 | Isovaleric acid-DTHFPCIKFEPRSKGWTCK-NH₂ | 13 |
| 183 | 206 | Isovaleric acid-DTHFPCIKFEPRSKGWCK-NH₂ | 17 |
| 184 | 207 | Isovaleric acid-DTHFPCIKFEPRSKGCK-NH₂ | 23 |
| 185 | 208 | Isovaleric acid-DTHFPCIFEPRSKGCK-NH₂ | 54 |
| 186 | 209 | Isovaleric acid-DTHFPCIFEPRSKGWCK-NH₂ | 12 |
| 187 | 210 | Isovaleric acid-DTHFPCIKFGPRSKCK-NH₂ | 21 |
| 188 | 211 | Isovaleric acid-DTHFPCIKFGPRSCK-NH₂ | 30 |
| 189 | 212 | Isovaleric acid-DTHFPCIKFGPRCK-NH₂ | 36 |
| 190 | 213 | Isovaleric acid-DTHFPCIKFGPCK-NH₂ | 55 |
| 191 | 214 | Isovaleric acid-DTHFPCIKFGCK-NH₂ | 97 |
| 192 | 215 | Isovaleric acid-DTHFPCIKFCK-NH₂ | 48 |
| 193 | 216 | Isovaleric acid-DTHFPCIKFC-NH₂ | 80 |
| 194 | 217 | Isovaleric acid-DTHFPCI-K(Palm)-FGPRSKGWVCK-NH₂ | 4 |
| 195 | 218 | Isovaleric acid-DTHFPCIKF-K(Palm)-PRSKGWVCK-NH₂ | 9 |
| 196 | 219 | Isovaleric acid-DTHFPCIKFGP-K(Palm)-SKGWVCK-NH₂ | 2 |
| 197 | 220 | Isovaleric acid-DTHFPCIKFGPRS-K(Palm)-GWVCK-NH₂ | 1 |
| 198 | 221 | Isovaleric acid-DTHFPCIKFGPRSKGWVC-K(Palm)-NH₂ | 7 |
| 199 | 222 | Isovaleric acid-DTHFPCI-K(PEG3-Palm)-FGPRSKGWVCK-NH₂ | 7 |
| 200 | 223 | Isovaleric acid-DTHFPCIKF-K(PEG3-Palm)-PRSKGWVCK-NH₂ | 6 |
| 201 | 224 | Isovaleric acid-DTHFPCIKFGP-K(PEG3-Palm)-SKGWVCK-NH₂ | 4 |
| 202 | 225 | Isovaleric acid-DTHFPCIKFGPRS-K(PEG3-Palm)-GWVCK-NH₂ | 3 |
| 203 | 226 | Isovaleric acid-DTHFPCIKFGPRSKGWVC-K(PEG3-Palm)-NH₂ | 4 |
| 204 | 227 | Hy-DTHFPCI-K(IVA)-FGPRSKGWVCK-NH₂ | >300 nM |
| 205 | 228 | Hy-DTHFPCIKF-K(IVA)-PRSKGWVCK-NH₂ | >300 nM |
| 206 | 229 | Hy-DTHFPCIKFGP-K(IVA)-SKGWVCK-NH₂ | 624 |
| 207 | 230 | Hy-DTHFPCIKFGPRS-K(IVA)-GWVCK-NH₂ | 318 |
| 208 | 231 | Hy-DTHFPCIKFGPRSKGWVC-K(IVA)-NH₂ | 109 |
| 209 | 232 | Hy-DTHFPCI-K(PEG3-IVA)-FGPRSKGWVCK-NH₂ | 342 |
| 210 | 233 | Hy-DTHFPCIKF-K(PEG3-IVA)-PRSKGWVCK-NH₂ | 457 |
| 211 | 234 | Hy-DTHFPCIKFGP-K(PEG3-IVA)-SKGWVCK-NH₂ | >300 nM |
| 212 | 235 | Hy-DTHFPCIKFGPRS-K(PEG3-IVA)-GWVCK-NH₂ | >300 nM |
| 213 | 236 | Hy-DTHFPCIKFGPRSKGWVC-K(PEG3-IVA)-NH₂ | 233 |
| 214 | 237 | Isovaleric acid-DTHFPCIKFEPRSKKWVCK-NH₂ | 15 |
| 215 | 238 | Hy-DTHFPCIKFGPRSKGWVCK-NH₂ | >1 μM |
| 216 | 239 | Palm-DTHFPCIKFGPRSKGWVCK-NH₂ | >1 μM |
| 217 | 240 | Palm-PEG3-DTHFPCIKFGPRSKGWVCK-NH₂ | >1 μM |
| 218 | 241 | Isovaleric acid-DTHFPCI-K(isoglu-Palm)-FEPRSKGCK-NH₂ | 10 |
| 219 | 242 | Isovaleric acid-DTHFPCIKF-K(isoglu-Palm)-PRSKGCK-NH₂ | 9 |
| 220 | 243 | Isovaleric acid-DTHFPCIKFEP-K(isoglu-Palm)-SKGCK-NH₂ | 5 |
| 221 | 244 | Isovaleric acid-DTHFPCIKFEPRS-K(isoglu-Palm)-GCK-NH₂ | 4 |
| 222 | 245 | Isovaleric acid-DTHFPCIKFEPRSK-K(isoglu-Palm)-CK-NH₂ | 4 |
| 223 | 246 | Isovaleric acid-DTHFPCIKFEPRSKGC-K(isoglu-Palm)-NH₂ | 5 |
| 224 | 247 | Isovaleric acid-DTHFPCIKFEPRSKGCK-K(isoglu-Palm)-NH₂ | 4 |
| 225 | 248 | Isovaleric acid-DTHFPCI-K(dapa-Palm)-FEPRSKGCK-NH₂ | 17 |
| 226 | 249 | Isovaleric acid-DTHFPCIKF-K(dapa-Palm)-PRSKGCK-NH₂ | 14 |
| 227 | 250 | Isovaleric acid-DTHFPCIKFEP-K(dapa-Palm)-SKGCK-NH₂ | 10 |
| 228 | 251 | Isovaleric acid-DTHFPCIKFEPRS-K(dapa-Palm)-GCK-NH₂ | 7 |
| 229 | 252 | Isovaleric acid-DTHFPCIKFEPRSK-K(dapa-Palm)-CK-NH₂ | 13 |
| 230 | 253 | Isovaleric acid-DTHFPCIKFEPRSKGC-K(dapa-Palm)-K-NH₂ | 10 |
| 231 | 254 | Isovaleric acid-DTHFPCIKFEPRSKGCK-K(dapa-Palm)-NH₂ | 11 |
| 232 | 255 | Isovaleric acid-DTHFPCIKFGPRSKGWVCK-NH₂ | Not Tested |
| 233 | 256 | Isovaleric acid-AAHFPCIKFGPRSKGWVCK-NH₂ | 320 |
| 234 | 257 | Isovaleric acid-ATHFPCIKFGPRSKGWVCK-NH₂ | 60 |
| 235 | 258 | Isovaleric acid-DAHFPCIKFGPRSKGWVCK-NH₂ | 203 |
| 236 | 259 | Isovaleric acid-DTHAPCIKFGPRSKGWVCK-NH₂ | >500 nM |
| 237 | 260 | Isovaleric acid-DTHFPCIKAGPRSKGWVCK-NH₂ | 50 |
| 238 | 261 | Isovaleric acid-DTHFPCIKFEPRSKGWVCK-OH | 47 |
| 239 | 262 | Isovaleric acid-DTHFPCIKFEPRSKGWECK-OH | 101 |
| 240 | 263 | Isovaleric acid-DTHFPCIIFEPRSKGWEC-OH | 139 |
| 241 | 264 | Isovaleric acid-DTHFPCIKFK(isoGlu-Palm)-PRSKGWECK-NH₂ | 6 |
| 242 | 265 | Isovaleric acid-DTHFPCIKFEPK(isoGlu-Palm)-SKGWECK-NH₂ | 8 |
| 243 | 266 | Isovaleric acid-DTHAPCIKFEPRSKGWECK-NH₂ | Inactive |
| 244 | 267 | Ida-THFPCIKFEPRSK-K(isoGlu-Palm)CK-NH₂ | 25 |
| 245 | 268 | Isovaleric acid-DTHFPCI-K(isoGlu-Palm)-FEPRSKGWEC-OH | 131 |
| 246 | 269 | 4,4-5,5-6,6,6-Heptafluorohexanoic acid-DTHFPCIKFGPRSKGWVCK-NH₂ | 480 |
| 247 | 270 | Isovaleric acid-DTHFPCIKF-K(mysteric acid)-PRSKGWVC-NH₂ | 7 |
| 248 | 271 | Isovaleric acid-DTHFPCIKF-K(lauric acid)-PRSKGWVC-NH₂ | 10 |
| 249 | 272 | Isovaleric acid-DTHFPCIKF-K(decanoic acid)-PRSKGWVC-NH₂ | 22 |
| 250 | 273 | Isovaleric acid-DTHFPCIKF-K(octanoic acid)-PRSKGWVC-NH₂ | 30 |
| 251 | 274 | Isovaleric acid-DTHFPCIKF-K(hexanoic acid)-PRSKGWVC-NH₂ | 21 |
| 252 | 275 | Isovaleric acid-DTHFPCIKF-K(butyric acid)-PRSKGWVC-NH₂ | 37 |
| 253 | 276 | Isovaleric acid-DTHFPCIKF-K(Ac)-PRSKGWVC-NH₂ | 29 |
| 254 | 277 | Ida-THFPCIKFEPRSKGWVC-K(mysteric acid)-NH₂ | 20 |
| 255 | 278 | [Ida]-THFPCIKFEPRSKGWVC-K(lauric acid)-NH₂ | 52 |
| 256 | 279 | [Ida]-THFPCIKFEPRSKGWVC-K(decanoic acid)-NH₂ | 116 |
| 257 | 280 | [Ida]-THFPCIKFEPRSKGWVC-K(octanoic acid)-NH₂ | 129 |
| 258 | 281 | [Ida]-THFPCIKFEPRSKGWVC-K(hexanoic acid)-NH₂ | 191 |
| 259 | 282 | [Ida]-THFPCIKFEPRSKGWVC-K(butyric acid)-NH₂ | 355 |
| 260 | 283 | [Ida]-THFPCIKFEPRSKGWVC-K(Ac)-NH₂ | 502 |
| 261 | 284 | Isovaleric acid-HFPCIKFEPRSKGWVC-K(octanoic acid)-NH₂ | >300 nM |
| 262 | 285 | Isovaleric acid-HFPCIKFEPRSKGWVC-K(lauric acid)-NH₂ | 77 |
| 263 | 286 | Isovaleric acid-DTHFPCIKFEPHSKGCK-NH₂ | 62 |
| 264 | 287 | Isovaleric acid-DTHFPCIHFEPHSKGC-NH₂ | 118 |
| 265 | 288 | Isovaleric acid-DTHFPCIKFEPHS-K(Albu)-GCK-NH₂ | 6 |
| 266 | 289 | Isovaleric acid-DTHFPCIKFEPREKEC-NH₂ | 183 |
| 267 | 290 | Isovaleric acid-DTAFPCIKFEPRSKEC-NH₂ | >1 μM |
| 268 | 291 | Isovaleric acid-DTHFPCIKFECK-NH₂ | 107 |
| 269 | 292 | Hy-DTHFPIAIFAAGICI-NH₂ | Inactive |
| 270 | 293 | Hy-DTHFPIAIFAAICI-NH₂ | Inactive |
| 271 | 294 | Hy-DTHFPIAIFAICI-NH₂ | Inactive |
| 272 | 295 | Hy-DTHFPIAIFICI-NH₂ | Inactive |
| 273 | 296 | Hy-DTHFPIAIICI-NH₂ | Inactive |
| 274 | 297 | Hy-DTHFPIAICI-NH₂ | Inactive |
| 275 | 298 | Hy-DTHFPIICI-NH₂ | Inactive |
| 276 | 299 | Hy-DTHICIAIF-NH₂ | Inactive |
| 277 | 300 | Hy-DTHCPIAIF-NH₂ | Inactive |
| 278 | 301 | Hy-DTHFPCIIA-NH₂ | >1 μM |
| 279 | 302 | Hy-DTHFPCAIF-NH₂ | >1 μM |
| 280 | 303 | Hy-DTHFACIIF-NH₂ | >1 μM |
| 281 | 304 | Hy-DTHF-(D)--Ala-CIIF-NH₂ | Inactive |
| 282 | 305 | Hy-DTHAPCIIF-NH₂ | Inactive |
| 283 | 306 | Hy-DTAFPCIIF-NH₂ | 739 nM |
| 284 | 307 | Hy-ATHFPCIIF-NH2 | >1 μM |
| 285 | 308 | [Ida]-THF-[bh-Pro]-CIIF-NH₂ | >1 μM |
| 287 | 310 | Hy-DTHFPCIEF-NH₂ | >1 μM |
| 288 | 311 | Isovaleric acid-DTHFPCIIF-NH₂ | 16 nM |
| 289 | 312 | Isovaleric acid-DTHFPAIIF-NH2 | Inactive |
| 290 | 313 | Isovaleric acid-DTHFPSIIF-NH2 | Inactive |
| 291 | 314 | Isovaleric acid-DTHFPCIKF-NH₂ | 7 nM |
| 293 | 316 | Hy-DTHFPCIF-NH₂ | 52% at 1 μM |
| 297 | 320 | Hy-DTHFPCIKFF-NH₂ | 64% at 1 μM |
| 298 | 321 | Hy-YTHFPCIIF-NH₂ | Not Tested |
| 299 | 322 | Hy-LTHFPCIIF-NH₂ | 64% at 1 μM |
| 300 | 323 | Hy-ETHFPCIIF-NH₂ | 77% at 1 μM |
| 301 | 324 | Hy-DRHFPCIIF-NH₂ | Not Tested |
| 302 | 325 | Hy-DTKFPCIIF-NH₂ | 60% at 1 μM |
| 303 | 326 | Hy-DTHFECIIF-NH₂ | Not Tested |
| 304 | 327 | Hy-DTHFPCIIK-NH₂ | 55% at 1 μM |
| 305 | 328 | Hy-DTHFPCIIR-NH₂ | 62% at 1 μM |
| 306 | 329 | Hy-DTHFPCIEF-NH₂ | Not Tested |
| 307 | 330 | Hy-DTHFPCIVF-NH₂ | 75% at 1 μM |
| 308 | 331 | Hy-DTHFPCILF-NH₂ | 89% at 1 μM |
| 309 | 332 | Hy-DTHFPCILK-NH₂ | 55% at 1 μM |
| 310 | 333 | Hy-DTHFPCIEK-NH₂ | 0% at 1 μM |
| 355 | 369 | Isovaleric acid-DTHFPCIKFEPRSKECK-NH₂ | 48 |
| 356 | 370 | Isovaleric acid-DTHFPCIKFEPHSKECK-NH₂ | 181 |
| 357 | 371 | Isovaleric acid-DTHFPCIKKEPHSKECK-NH₂ | >1 μM |
| 358 | 372 | Isovaleric acid-DTHFPCIKF-K(isoglu-Palm)-PHSKECK-NH₂ | 6 |
| 359 | 373 | Isovaleric acid-DTHFPCIKFEPRECK-NH₂ | 64 |
| 360 | 374 | Isovaleric acid-DTHFPCIKFEPHECK-NH₂ | 138 |
| 361 | 375 | Isovaleric acid-DTHFPCIKFEPRCK-NH₂ | 29 |

Inactive = Not active at 30 μM and/or lowest dose.
For Table 7, parentheticals, e.g., (_), represent side chain conjugations and brackets, e.g., [_], represent unnatural amino acid substitutions.

For certain compounds comprising an N-terminal PEG11 moiety (e.g., compounds 89, 108, and 109), the following was used in their synthesis:

### Fmoc-Amino PEG Propionic Acid

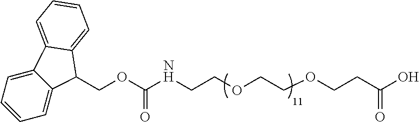

### Example 6

### Alanine Scan of Compound 18

To further understand Hepcidin's structure activity relationship, an alanine scan was performed on Compound 18, which is a Hepcidin analogue of the present invention that comprises a cysteine in the 7 position. Peptides were synthesized as described in Example 1 and tested for activity as described in Example 2; results are shown in Table 8 herein

TABLE 8
Alanine scan of Coupound 18

| | | | EC50 |
|---|---|---|---|
| Compound | SEQ | | (nM) |
| Number | ID No. | Sequence | (n > 3) |
| 18 | 45 | DTHFPICIFGPRSKGWVCM-NH₂ | 125 |
| 47 | 74 | DTHFPICIFGPRSKGWACM-NH₂ | 313 |
| 66 | 89 | DTHFPICIFGPRSKGAVCM-NH₂ | 520 |
| 31 | 58 | DTHFPICIFGPRSKAWVCM-NH₂ | 175 |
| 37 | 64 | DTHFPICIFGPRSAGWVCM-NH₂ | 239 |
| 16 | 43 | DTHFPICIFGPRAKGWVCM-NH₂ | 88 |
| 34 | 61 | DTHFPICIFGPASKGWVCM-NH₂ | 206 |
| 354 | 334 | DTHFPICIFGARSKGWVCM-NH₂ | 153 |
| 22 | 49 | DTHFPICIFAPRSKGWVCM-NH₂ | 9430 |
| 67 | 90 | DTHFPICIAGPRSKGWVCM-NH₂ | 2466 |
| 68 | 91 | DTHFPICAFGPRSKGWVCM-NH₂ | >10 μM |
| 69 | 92 | DTHFPIAIFGPRSKGWVAM-NH₂ | Inactive |
| 29 | 56 | DTHFPACIFGPRSKGWVCM-NH₂ | 157 |
| 26 | 53 | DTHFAICIFGPRSKGWVCM-NH₂ | 147 |
| 38 | 65 | DTHAPICIFGPRSKGWVCM-NH₂ | 254 |
| 60 | 87 | DTAFPICIFGPRSKGWVCM-NH₂ | 498 |
| 43 | 70 | DAHFPICIFGPRSKGWVCM-NH₂ | 265 |
| 33 | 60 | ATHFPICIFGPRSKGWVCM-NH₂ | 184 |

Inactive = Not active at 30 μM and/or lowest dose

As was the case with the alanine scan of compound I (cysteine in position 6) this scan identified residues within compound 18 (cysteine in position 7) that are important for activity, as well as several residues that appear to be less important for activity and thus may modified without ablating activity.

### Example 7

### Plasma Stability

Serum stability experiments were undertaken to complement the in vivo results and assist in the design of potent, stable Ferroportin agonists. In order to predict the stability in humans, ex vivo stability studies were initially performed in human serum.

Key peptides (10 μM) were incubated with pre-warmed human serum (Sigma) at 37 degrees C. Samples were taken at various time points up to 24 hours. The samples were separated from serum proteins and analysed for the presence of the peptide of interest using LC-MS. The amount of intact peptide in each sample was calculated using the analyte peak area in relation to the zero time point. Table 9 shows the results of this study.

TABLE 9
Stability of key compounds in human serum

| Compound No. | t½ (h) |
|---|---|
| Hepcidin | 2.76 |
| Mini Hepcidin 1-9 | 0.10 |
| 1 | 0.18 |
| 18 | 2.32 |
| 46 | 2.10 |
| 2 | 1.99 |
| 47 | ~40 |
| 8 | 0.51 |
| 3 | 0.51 |

### Example 8

### Reduction of Free Plasma Iron in Rats

To investigate whether the hepcidin mimetic Compound No. 2 was effective in decreasing free Fe²⁺ in serum, Retro Inverse mini Hepcidin was used as a reference peptide. Although RI mini-Hep has a very low potency in vitro it is highly active in vivo as reported by Presza et al. J Clin Invest. 2011.

At Day 1, the animals were monitored for free Fe²⁺ in serum. In order to reach a homogenous serum level, Fe²⁺ was analyzed and a homogenous cohort of 7 or 8 animals randomized to each treatment group. A Day 2, an acute experiment where the animals were subjected to i.p. dosing of test compound and subsequent tail vein blood samples. Prior to dosing, the animals were put under a heating lamp for 3-5 minutes. Blood samples were drawn from the tail vein from all animals in order to determine serum iron levels prior to vehicle or compound dosing. Animals were dosed i.p. with 1 ml of test substance in vehicle or just vehicle and blood samples of 250 μl was drawn from each animal at t=0, 60, 120, 240, 360 min and 24 hours in the study of the reference compound. The dose response study performed with Retro Inverse (RI) mini-Hepcidin (Reference compound), and the efficacy study performed with Compound No. 2 were performed as two separate experiments.

Analysis of Fe²⁺ from Day 0 and 1 was done at a later time point not later than 10 days after. The chemicals and equipment used in this example are shown in Table 10.

TABLE 10
Chemicals and equipment used in this example

| | | SEQ | | Peptide | Peptide | | |
|---|---|---|---|---|---|---|---|
| | Cmpd. | ID | MW | Content | Content | | |
| Drug Name | No. | No. | (g/mol) | Calculated % | Determined % | Purity % | Solvent |
| Isovaleric | 2 | 29 | 2144.52 | 86.2 | 86.2 | 90 | Na- |
| acid- | | | | | | | Acetate |
| DTHFPICIF | | | | | | | buffer |
| GPRSKGW | | | | | | | |
| VC-NH₂ | | | | | | | |
| RI- | | 337 | 1091.3 | 82.7 | 82.7 | 94.2 | Strong |
| Hepcidin1-9 | | | | | | | PBS |

Initially, all peptides were solubilized in acidic H2O in pH=2.5 and to a concentration of 3 mg/ml API. Compounds were thereafter either dissolved in Na-Acetate buffer (50 mM Acetic Acid, 125 mM NaCl, pH 5.0) or strong PBS, (25 mM sodium phosphate, 125 mM NaCl, pH 7.4).

Male Sprague-Dawley rats weighing 200-250 g were used in the study. They were housed in groups for n=2 in a light-, temperature- and humidity-controlled room (12-hour light: 12-hour dark cycle, lights on/off at 0600/1800 hour; 23 degrees Celcius; 50% relative humidity). Humane endpoints were applied, according to OECD's ‘Guidelines for Endpoints in Animal Study Proposals.” The animals were monitored daily. In case of significantly affected condition (based on signs such as weight loss>30% (obese animals); abnormal posture; rough hair coat; exudate around eyes and/or nose; skin lesions; abnormal breathing; difficulty with ambulation; abnormal food or water intake; or self mutilation), or other conditions causing significant pain or distress, the animals were euthanized immediately.

Iron content in plasma/serum is measured for iron content using a colorimetric assay on the Cobas c 111 according to instructions from the manufacturer of the assay (assay: IRON2: ACN 661)

The data obtained from the cobas Iron2 analysis are presented as mean values +/−SEM.

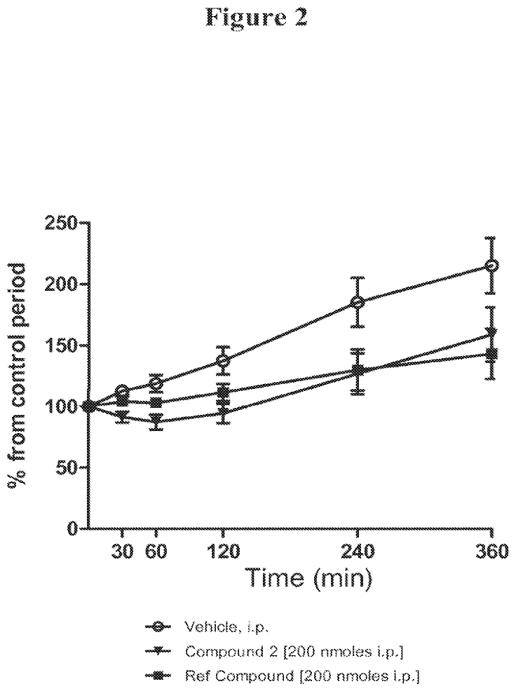

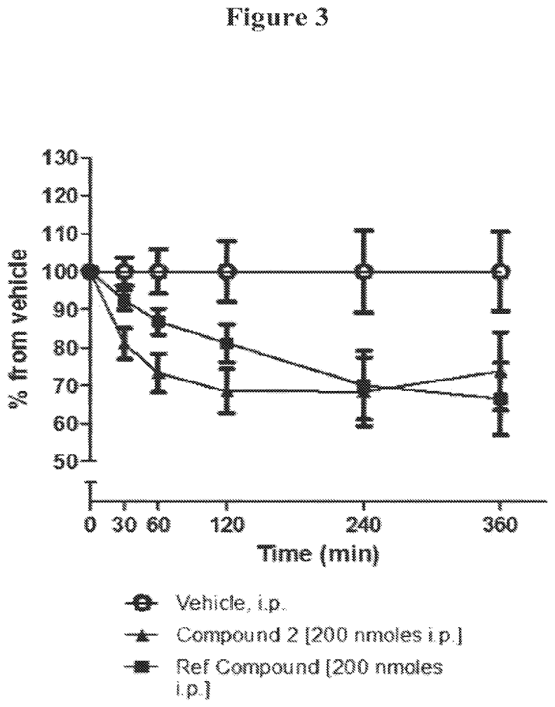

As shown in FIGS. 2 and 3, IP dosing of compound 2 resulted in a decrease in serum iron level that was comparable to that observed after injection of the positive control Retro Inverse mini Hepcidin (RI-Mini-Hepcidin). The decrease induced by RI-Mini-Hepcidin and compound 2 was in neither case significant, which was probably due to a large intergroup variance in the measurements.

### Example 9

### In Vito Validation of Selected Peptides

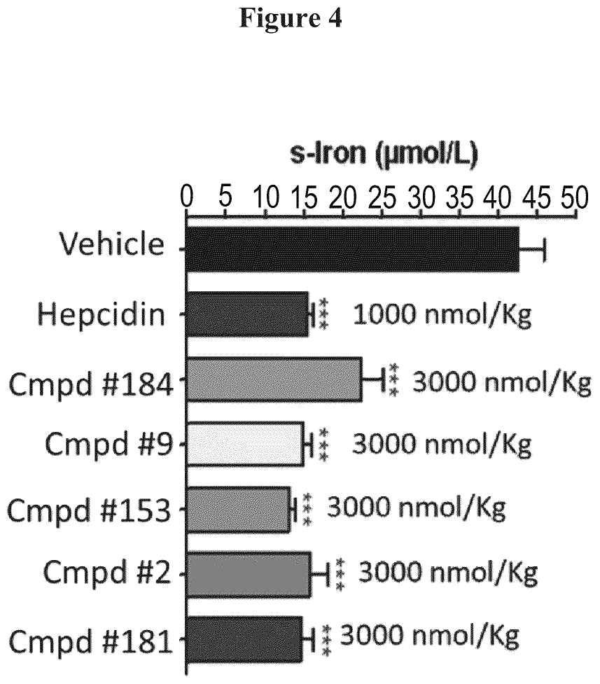

Selected peptides of the present invention were tested for in vivo activity, as described in Example 8, with the following changes. Instead of rats, mice (C57-BL6) were tested. Peptides or vehicle controls were administered to the mice (n=8/group) with the compounds of the present invention dosed at 3000 nmol/kg, and a hepcidin control administered via subcutaneous injection at 1000 nmol/kg. The primary goal of this experiment was to validate, in a mouse model, the activity of several peptides that were shown to be active in rat. Serum iron levels were assessed as in Example 8 two hours after peptide or vehicle administration. As shown in FIG. 4, at these doses, a significant reduction in serum iron was observed in compound-treated animals as compared to the vehicle control. Furthermore, the max-dose responses of the compounds of the invention were very similar to the max-dose response achieved with Hepcidin.

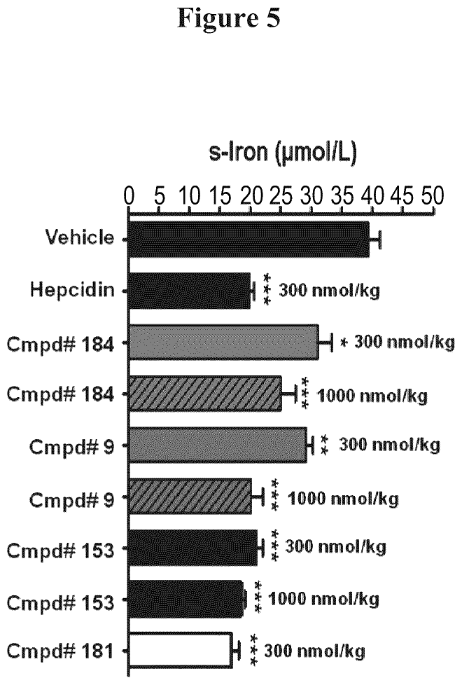

A similar experiment was performed with lower doses to assess the dose response of these compounds for inducing serum iron reduction. Methods were as described above in this Example, except for the following parameters: n=4 mice/group, however n=8 for the vehicle, as two groups were pooled. Mice were administered test compounds at two separate dosages (300 nmol/kg or 1000 nmol/kg), via subcutaneous injection. Serum iron levels were assessed as in Example 8 two hours after peptide or vehicle injection. As shown in FIG. 5, these peptides induced similar iron reductions as did native hepcidin in vivo. Moreover, it was clear that several of the compounds were able to induce maximum effects at dosages as low as 300 nmol/kg.

Other peptides were tested similarly, either in rats as described in Example 8, or in mice as described above in the present Example, and the results of these tests are presented in Table 11, herein, in the column having the heading “in vivo activity.” In this table, dosing is indicated in the sub-headings listed in the first row of the “in vivo activity” column; in vivo activity data is reported as a “yes” or “no” determination, with yes indicating that in vivo activity for serum iron reduction was observed, and with “no” indicating that no such activity was observed. The route of peptide administration was via subcutaneous injection, unless otherwise indicated as having been via intraperitoneal injection (this is noted on the table by “i.p.” in parentheses following the “yes” or “no” determination).

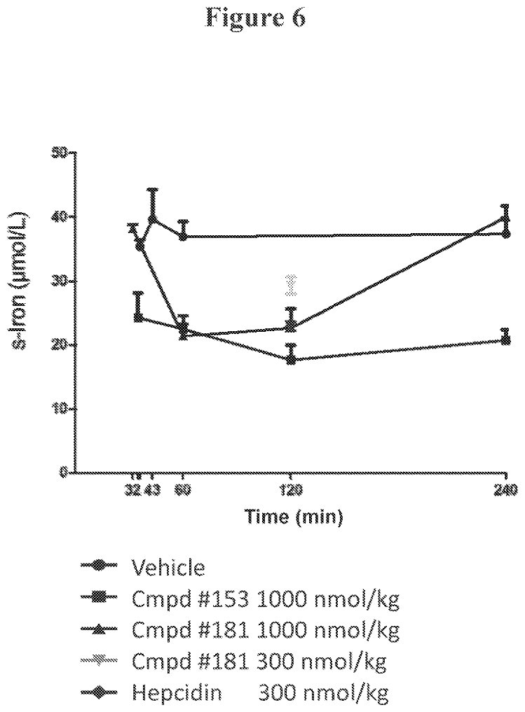

The peptides were also tested for other pharmacokinetic/pharmacodynamic (PK/PD) parameters using methods commonly know by the skilled artisan. The results of these tests are also indicated on Table 11. These parameters included determinations regarding stability (hours stable in plasma from the indicated human or rat subject), half-life in mice, and in vitro activity (EC₅₀), tested as described in Example 2. One example of such a study is presented in FIG. 6, wherein the PK/PD properties of two compounds of the present invention (#153 and #181) were compared with hepcidin to determine their PK/PD effects in C57BL6 mice. Each of these compounds produced a rapid decrease in serum iron, which was transient in the case of Cmpd #181, and sustained in the case of Cmpd #153.

These data, in addition to the data presented herein in Table 11, demonstrated the activity and beneficial PK/PD properties of the peptides of the present invention, e plurality of which show similar or improved PK/PD profiles as compared to hepcidin.

TABLE 11
Peptide activities in vivo

| | | | | | | In Vivo | |
|---|---|---|---|---|---|---|---|
| | | | | Mouse | In Vitro | Activity (s.c.) | |
| | | Stability | | PK T_{1/2} | Activity | 300 | 1000 |
| Cmpd No | Sequence | Rat | Human | (min) | EC50 (nM) | nmol/kg | nmol/kg |
| Hepcidin | Hy-DTHFPICIFCCGC | Var | 2.76 | | 34 | Yes | Yes |
| | CHRSKCGMCCKT-OH | | | | | | |
| | SEQ ID NO: 335 | | | | | | |
| 2 | Isovaleric acid- | 0.15 | 1.99 | 17.4 | 5 | Yes | Yes |
| | DTHFPICIFGPRSKGWVC- | | | | | | |
| | NH₂ (SEQ ID NO: 29) | | | | | | |
| 3 | Isovaleric acid- | 0.08 | 0.43 | | 15 | No | No |
| | DTHFPCIIFGPRSRGWVCK- | | | | | (i.p.) | (i.p.) |
| | NH₂ (SEQ ID NO: 30) | | | | | | |
| 105 | Isovaleric acid- | 0.68 | 2.22 | 36.9 | 10 | Yes | Yes |
| | DTHFPCIIFEPRSKGWVCK- | | | | | | |
| | NH₂ (SEQ ID NO: 128) | | | | | | |
| 9 | Isovaleric acid- | 0.14 | 0.57 | 22.5 | 32 | Yes | Yes |
| | DTHFPCIKFGPRSKGWVCK- | | | | | | |
| | NH₂ (SEQ ID NO: 36) | | | | | | |
| 10 | Isovaleric acid- | 0.12 | | | 35 | — | Minor |
| | DTHFPCIQFGPRSKGWVCK- | | | | | | |
| | NH₂ (SEQ ID NO: 37) | | | | | | |
| 15 | Isovaleric acid- | 0.15 | | | 79 | — | Minor |
| | DTHFPICIEFGPRSKG | | | | | | |
| | WVCK-NH₂ | | | | | | |
| | (SEQ ID NO: 42) | | | | | | |
| 115 | Isovaleric acid- | | | | 21 | — | No |
| | DTHFPCIIFGPRSKGCK- | | | | | | |
| | NH₂ (SEQ ID NO: 138) | | | | | | |
| 150 | Isovaleric acid- | 0.42 | 1.35 | 31.6 | 7 | Yes | |
| | DTHFPCIKFK(PEG8) | | | | | | |
| | PRSKGWVCK-NH₂ | | | | | | |
| | (SEQ ID NO: 173) | | | | | | |
| 153 | Isovaleric acid- | 0.41 | 3.36 | | 18 | Yes | Yes |
| | DTHFPCIKFGPRSKGW | | | | | | |
| | VCK(PEG8)-NH₂ | | | | | | |
| | (SEQ ID NO: 176) | | | | | | |
| 176 | Isovaleric acid- | 1.62 | 15 | | 6 | Yes | |
| | DTHFPICIFGPRSK | | | | | | |
| | (PEG8)GWVC-NH₂ | | | | | | |
| | (SEQ ID NO: 199) | | | | | | |
| 184 | Isovaleric acid- | 2.12 | 8.16 | 36.9 | 16 | Yes | Yes |
| | DTHFPCIKFEPRSKGCK- | | | | | | |
| | NH₂ (SEQ ID NO: 207) | | | | | | |
| 181 | Isovaleric acid- | | | | 15 | Yes | |
| | DTHFPCIKFEPRSKGWVCK- | | | | | | |
| | NH₂ (SEQ ID NO: 204) | | | | | | |

Unless otherwise stated all compounds were injected s.c.
Note
Compound 2 was injected I.P.

### Example 10

### In Vitro Activity of Selected Peptide Dimers

Selected peptide dimers of the present invention were tested for in vitro activity, as described in Example 2.

The EC₅₀ and % activity at 1 μM were determined for the peptide monomers and peptide dimers shown in Table 12. These peptide dimers were dimerized via a single disulphide linkage between a cysteine residue present in each peptide monomer. The results of these experiments are shown in Table 12.

TABLE 12
In vitro activity of peptides
dimerized through a single disulphide
linkage between cysteine residues

| | | EC₅₀ | % |
|---|---|---|---|
| Cmpd | | (nM) | Activity |
| # | Sequence | (n = 3) | At 1 uM |
| 1 | Hy-DTHFPCIIF-NH₂ | 133 | 92 |
| | (SEQ ID NO: 28) | | |
| 293 | Hy-DTHFPCIF-NH₂ | >1 μM | 52 |
| | (SEQ ID NO: 316) | | |
| 297 | Hy-DTHFPCIKFF-NH₂ | >300 nM | 64 |
| | (SEQ ID NO: 320) | | |
| 299 | Hy-LTHFPCIIF-NH₂ | >300 nM | 64 |
| | (SEQ ID NO: 322) | | |
| 300 | Hy-ETHFPCIIF-NH₂ | >300 nM | 77 |
| | (SEQ ID NO: 323) | | |
| 302 | Hy-DTKFPCIIF-NH2 | >1 μM | 60 |
| | (SEQ ID NO: 325) | | |
| 304 | Hy-DTHFPCIIK-NH₂ | >1 μM | 55 |
| | (SEQ ID NO: 327) | | |
| 305 | Hy-DTHFPCIIR-NH₂ | >1 μM | 62 |
| | (SEQ ID NO: 328) | | |
| 307 | Hy-DTHFPCIVF-NH₂ | >300 nM | 75 |
| | (SEQ ID NO: 330) | | |
| 308 | Hy-DTHFPCILF-NH₂ | >300 nM | 89 |
| | (SEQ ID NO: 331) | | |
| 309 | Hy-DTHFPCILK-NH₂ | >300 nM | 55 |
| | (SEQ ID NO: 332) | | |
| 310 | Hy-DTHFPCIEK-NH₂ | >1 μM | 0 |
| | (SEQ ID NO: 333) | | |
| 288 | Isovaleric acid- | 16 | 100 |
| | DTHFPCIIF-NH₂ | | |
| | (SEQ ID NO: 311) | | |
| 291 | Isovaleric acid- | 7 | 100 |
| | DTHFPCIKF-NH₂ | | |
| | (SEQ ID NO: 314) | | |
| 311 | (Hy-DTHFPCIIF-NH₂)₂ | 35 | 96 |
| | (SEQ ID NO: 338) | | |
| 312 | (Hy-DTHFPCI_F-NH₂)₂ | >300 nM | 51 |
| | (SEQ ID NO: 339) | | |
| 314 | (Hy-DTHFPCIKFF-NH₂)₂ | 130 | 100 |
| | (SEQ ID NO: 341) | | |
| 315 | (Hy-LTHFPCIIF-NH₂)₂ | 35 | 97 |
| | (SEQ ID NO: 342) | | |
| 316 | (Hy-ETHFPCIIF-NH₂)₂ | 63 | 100 |
| | (SEQ ID NO: 343) | | |
| 317 | (Hy-DTKFPCIIF-NH₂)₂ | 137 | 87 |
| | (SEQ ID NO: 344) | | |
| 318 | (Hy-DTHFPCIIK-NH₂)₂ | >300 nM | 49 |
| | (SEQ ID NO: 345) | | |
| 319 | (Hy-DTHFPCIIR-NH₂)₂ | 268 | 79 |
| | (SEQ ID NO: 346) | | |
| 320 | (Hy-DTHFPCIVF-NH₂)₂ | 50 | 93 |
| | (SEQ ID NO: 347) | | |
| 321 | (Hy-DTHFPCILF-NH₂)₂ | 83 | 94 |
| | (SEQ ID NO: 348) | | |
| 322 | (Hy-DTHFPCILK-NH₂)₂ | >300 nM | 47 |
| | (SEQ ID NO: 349) | | |
| 323 | (Hy-DTHFPCIEK-NH₂)₂ | >1 μM | 0 |
| | (SEQ ID NO: 350) | | |
| 325 | (Isovaleric acid- | 4 | 100 |
| | DTHFPCIIF-NH₂)₂ | | |
| | (SEQ ID NO: 351) | | |
| 326 | (Isovaleric acid- | 3 | 100 |
| | DTHFPCIKF-NH₂)₂ | | |
| | (SEQ ID NO: 352) | | |

EC₅₀ values were also determined for the peptide dimers having the sequences shown in Table 10. The activity of peptide dimers dimerized only through a disulphide linkage between the two peptide monomers was compared to the activity of peptide dimers of the same monomers dimerized through both the disulphide linkage and also a DIG linking moiety. In addition, the activity of peptide dimers dimerized through a DIG linking moiety coupled to the N-terminus of the monomers, the C-terminus of the monomers, or different internal lysine residues was examined. The results of these experiments are provided in Table 13.

TABLE 13
Dimer Position explored (DIG as the representative linker explored)

| | | EC₅₀ |
|---|---|---|
| Cmpd | | (nM) |
| No. | Sequence | (n > 3) |
| 327 (SEQ ID NO: 353) | 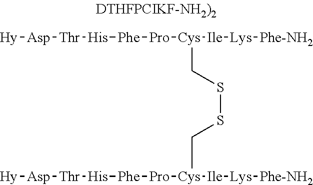 | 193 |
| 328 (SEQ ID NO: 354) | 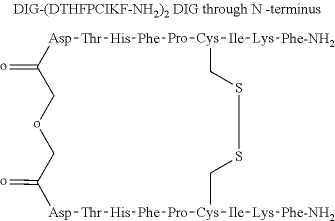 | >1000 |
| 329 (SEQ ID NO: 355) | 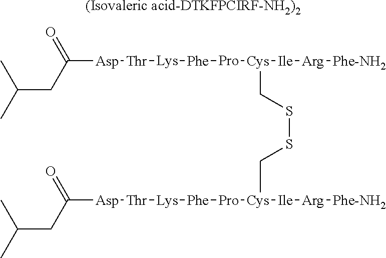 | 9 |
| 340 (SEQ ID NO: 356) | 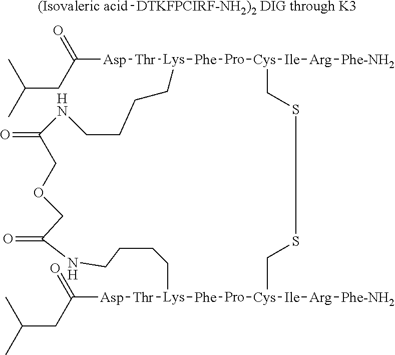 | 212 |
| 326 (SEQ ID NO: 357) | 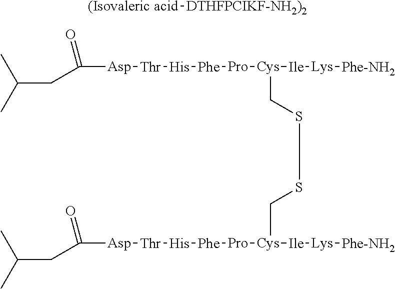 | 3 |
| 342 (SEQ ID NO: 358) | 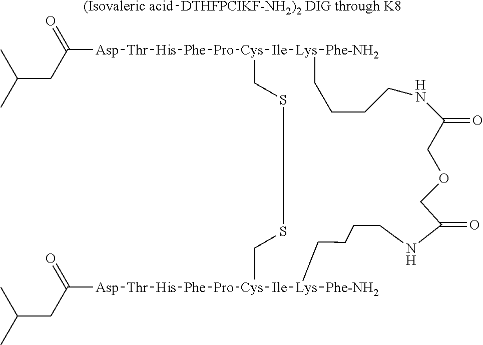 | 10 |
| 343 (SEQ ID NO: 359) | 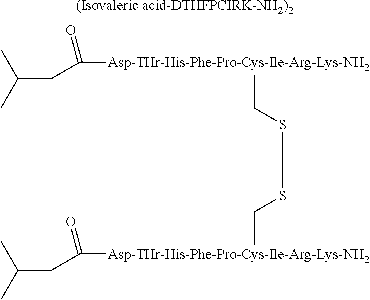 | 11 |
| 344 (SEQ ID NO: 360) | 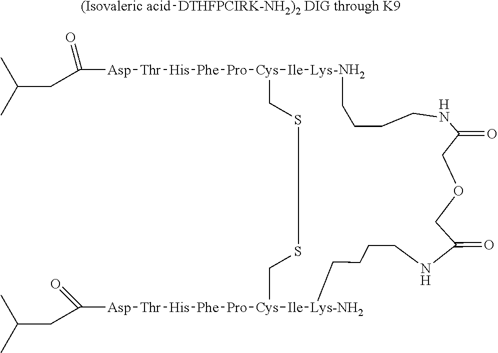 | 45 |
| 345 (SEQ ID NO: 361) | 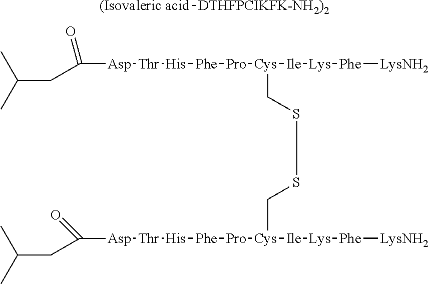 | 8 |
| 346 (SEQ ID NO: 362) | 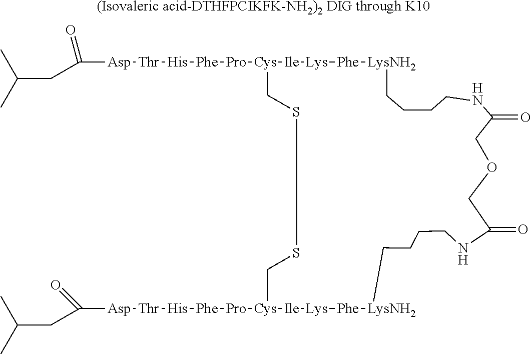 | 15 |

EC₅₀ values were determined for peptide dimers comprising different linking moieties, and as compared to linkage via a disulphide bridge between the two peptide monomers,

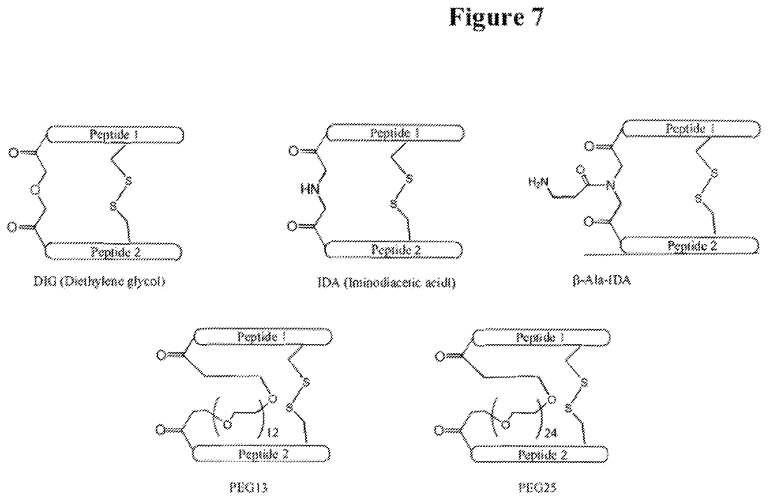

EC₅₀ values were determined for peptide dimers comprising different linking moieties, and as compared to linkage via a disulphide bridge between the two peptide monomers, including the peptide dimers shown in Table 14. Where a particular linking moiety is not indicated, the peptide dimer was dimerized via a disulphide bridge between cysteine residues present in each of the peptide monomers of the peptide dimer. The results of this experiment are shown in Table 14, and various linker types are shown as schematics in FIG. 7.

TABLE 14
Dimerization using various
linkers at different positions

| | | Log |
|---|---|---|
| | | dilutions |
| Cmpd. | | EC₅₀ (nM) |
| No. | Sequence | (n > 3) |
| 327 | (Hy-DTHFPCIKF-NH₂)₂ | 193 |
| | (SEQ ID NO: 353) | |
| 348 | (Hy-DTHFPCIKF-NH₂)₂-[IDA-(β-Ala)] | 18 |
| | (SEQ ID NO: 363) | |
| 326 | (Isovaleric acid-DTHFPCIKF-NH₂)₂ | 6 |
| | (SEQ ID NO: 357) | |
| 349 | (Isovaleric acid-DTHFPCIKF-NH₂)₂- | 5 |
| | [IDA-(β-Ala)-Palm] | |
| | (SEQ ID NO: 364) | |
| 345 | (Isovaleric acid-DTHFPCIKFK-NH₂)₂ | 8 |
| | (SEQ ID NO: 361) | |
| 346 | (Isovaleric acid-DTHFPCIKFK- | 15 |
| | NH₂)₂-[DIG] | |
| | DIG through K10 (SEQ ID NO: 362) | |
| 327 | (Hy-DTHFPCIKF-NH₂)₂ | 193 |
| | (SEQ ID NO: 353) | |
| 351 | [PEG25]-(DTHFPCIKF-NH₂)₂ | >1000 |
| | (SEQ ID NO: 366) | |

In Table 14, brackets indicate linker and any linker conjugates (if present), e.g., [linker].

EC₅₀ values were determined for peptide dimers dimerized via a glycol linker attached to the εN of lysine residues within the peptide chains, as compared to the peptide monomers. As shown in Table 15, the peptide dimers had lower EC₅₀s than their corresponding peptide monomers. In this case, the disulphide bond exists intramolecularly within each peptide (e.g., cmpd #2 and cmpd #3) moiety before dimerization through using DIG through acylation of the Nε of lysine.

TABLE 15
Dimerization through a glycol linker attached
to the ε_{N} of lysine within the peptide chain

| | | Log |
|---|---|---|
| | | dilutions |
| Cmpd | | EC50 (nM) |
| No. | Sequence | (n > 3) |
| 3 | Isovaleric acid- | 15 |
| | DTHFPCIIFGPRSRGWVCK-NH₂ | |
| | (SEQ ID NO: 30) | |
| 352 | (Isovaleric acid- | 5 |
| | DTHFPCIIFGPRSRGWVCK-NH₂)₂-[DIG] | |
| | (SEQ ID NO: 367) | |
| 2 | Isovaleric acid- | 4.1 |
| | DTHFPICIFGPRSKGWVC-NH₂ | |
| | (SEQ ID NO: 29) | |
| 353 | (Isovaleric acid- | 2.2 |
| | DTHFPICIFGPRSKGWVC-NH₂)₂-[DIG] | |
| | (SEQ ID NO: 368) | |

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method of treating a disease of iron metabolism in a subject, comprising administering to the subject an effective amount of at least one peptide according to formula I′:
(I′)
(SEQ ID NO: 21)
R1′-X′-Y′-R2′
or a pharmaceutically acceptable salt thereof, wherein
R1′ is hydrogen, a C1-C6 alkyl, a C6-C12 aryl, a C1-C20 alkanoyl or pGlu;
R2′ is —NH₂ or —OH;
X′ is a peptide sequence having the formula Ia′
(Ia′)
(SEQ ID NO: 13)
X1-X2-X3-X4-X5-X6-X7-X8-X9-X10
wherein
X1 is Asp, Ala, Ida, pGlu, bhAsp, Leu, D-Asp or absent;
X2 is Thr, Ala, or D-Thr;
X3 is His, Lys, D-His or Lys;
X4 is Phe, Ala, Dpa or D-Phe;
X5 is Pro, Gly, Arg, Lys, Ala, D-Pro or bhPro;
X6 is IIe, Cys, Arg, Lys, D-Ile or D-Cys;
X7 is Cys, IIe, Leu, Val, Phe, D-Ile or D-Cys;
X8 is IIe, Arg, Phe, Gln, Lys, Glu, Val, Leu or D-Ile;
X9 is Phe or bhPhe; and
X10 is Lys, Phe or absent;
wherein if Y′ is absent, X7 is Ile; and
Y′ is a peptide sequence having the formula IIa′
(IIa′)
(SEQ ID NO: 16)
Y1-Y2-Y3-Y4-Y5-Y6-Y7-Y8-Y9-Y10-Y11-Y12-Y13-Y14-Y15
wherein
Y1 is Gly, Cys, Ala, Phe, Pro, Glu, Lys, D-Pro, Val, Ser or absent;
Y2 is Pro, Ala, Cys, Gly or absent;
Y3 is Arg, Lys, Pro, Gly, His, Ala, Trp or absent;
Y4 is Ser, Arg, Gly, Trp, Ala, His, Tyr or absent;
Y5 is Lys, Met, Arg, Ala or absent;
Y6 is Gly, Ser, Lys, IIe, Ala, Pro, Val or absent;
Y7 is Trp, Lys, Gly, Ala, IIe, Val or absent;
Y8 is Val, Thr, Gly, Cys, Met, Tyr, Ala, Glu, Lys, Asp, Arg or absent;
Y9 is Cys, Tyr or absent;
Y10 is Met, Lys, Arg, Tyr or absent;
Y11 is Arg, Met, Cys, Lys or absent;
Y12 is Arg, Lys, Ala or absent;
Y13 is Arg, Cys, Lys, Val or absent;
Y14 is Arg, Lys, Pro, Cys, Thr or absent; and
Y15 is Thr, Arg or absent;
wherein the peptide of formula I′ comprises two cysteine residues linked via a disulfide bond,
wherein the peptide is optionally PEGylated on R1′, X′, or Y′;
wherein a side chain of an amino acid of the peptide is optionally conjugated to a lipophilic substituent or a polymeric moiety; and
wherein the disease of iron metabolism is hereditary hemochromatosis, iron hemochromatosis, HFE (human factors engineering) mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, or β-thalassemia.

2. The method of claim 1, wherein the peptide of Formula I′ is PEGylated on R1′, X′, or Y′, or wherein a side chain of an amino acid of the peptide of Formula I′ is conjugated to a lipophilic substituent or a polymeric moiety.

3. The method of claim 1, wherein R1′ is hydrogen, isovaleric acid, isobutyric acid or acetyl.

4. The method of claim 1, wherein X′ is a peptide sequence having formula Ib′
(Ib′)
(SEQ ID NO: 14)
X1-Thr-His-X4-X5-X6-X7-X8-Phe-X10
wherein
X1 is Asp, Ida, pGlu, bhAsp or absent;
X4 is Phe or Dpa;
X5 is Pro or bhPro;
X6 is IIe, Cys or Arg;
X7 is Cys, IIe, Leu or Val;
X8 is IIe, Lys, Glu, Phe, Gln or Arg; and
X10 is Lys or absent;
or having formula Ic′
(Ic′)
(SEQ ID NO: 15)
X1-Thr-His-X4-X5-Cys-Ile-X8-Phe-X10
wherein
X1 is Asp, Ida, pGlu, bhAsp or absent;
X4 is Phe or Dpa;
X5 is Pro or bhPro;
X8 is IIe, Lys, Glu, Phe, Gln or Arg; and
X10 is Lys or absent.

5. The method of claim 1, wherein X1 is Asp, X4 is Phe, and X5 is Pro.

6. The method of claim 1, wherein the peptide of Formula I′ comprises one of the following sequences or structures:
(SEQ ID NO: 29)
DTHFPICIFGPRSKGWVC;
(SEQ ID NO: 38)
DTHFPCIIFGPRSKGWVCK;
(SEQ ID NO: 128)
DTHFPCIIFEPRSKGWVCK;
(SEQ ID NO: 164)
DTHFPCIIFGPRSKGWACK;
(SEQ ID NO: 168)
DTHFPCIIFGPRSKGWVCKK;
(SEQ ID NO: 57
DTHFPCIIFVCHRPKGCYRRVCR;
(SEQ ID NO: 125)
DTHFPCIKFGPRSKGWVCK;
(SEQ ID NO: 173)
DTHFPCIKFKPRSKGWVCK;
(SEQ ID NO: 106)
DTHFPCIIFGPRSRGWVCK;
(SEQ ID NO: 135)
DTHFPCIKFGPKSKGWVCK;
(SEQ ID NO: 207)
DTHFPCIKFEPRSKGCK;
(SEQ ID NO: 265)
DTHFPCIKFEPKSKGWECK;
(SEQ ID NO: 245)
DTHFPCIKFEPRSKKCK;
(SEQ ID NO: 247)
DTHFPCIKFEPRSKGCKK;
(SEQ ID NO: 249)
DTHFPCIKFKPRSKGCK;
or
(SEQ ID NO: 250)
DTHFPCIKFEPKSKGCK,
or a pharmaceutically acceptable salt thereof.

7. The method of claim 1, wherein the peptide of Formula I′ comprises one of the following sequences or structures:
(SEQ ID NO: 29)
Isovaleric acid-DTHFPICIFGPRSKGWVC-NH₂;
(SEQ ID NO: 38)
Isovaleric acid-DTHFPCIIFGPRSKGWVCK-NH₂;
(SEQ ID NO: 128)
Isovaleric acid-DTHFPCIIFEPRSKGWVCK-NH₂;
(SEQ ID NO: 164)
Isovaleric acid-DTHFPCIIFGPRSKGWACK-NH₂;
(SEQ ID NO: 168)
Isovaleric acid-DTHFPCIIFGPRSKGWVCKK-NH₂;
(SEQ ID NO: 57)
Isovaleric acid-DTHFPCIIFVCHRPKGCYRRVCR-NH₂;
(SEQ ID NO: 172)
Isovaleric acid-DTHFPCI(K(PEG8))FGPRSKGWVCK-NH₂;
(SEQ ID NO: 173)
Isovaleric acid-DTHFPCIKF(K(PEG8))PRSKGWVCK-NH₂;
(SEQ ID NO: 199)
Isovaleric acid-DTHFPICIFGPRS(K(PEG8))GWVCNH₂;
(SEQ ID NO: 200)
Isovaleric acid-DTHFPICIFGPRS(K(PEG4))GWVCNH₂;
(SEQ ID NO: 201)
Isovaleric acid-DTHFPCIIFGPRSRGWVC(K(PEG8))-NH₂;
(SEQ ID NO: 202)
Isovaleric acid-DTHFPCIIFGPRSRGWVC(K(PEG4))-NH₂;
(SEQ ID NO: 203)
Isovaleric acid-DTHFPCIIFGPRSRGWVC(K(PEG2))-NH₂;
(SEQ ID NO: 183)
Isovaleric acid-DTHFPCI(K(Palm))FGPRSKGWVCK-NH₂;
(SEQ ID NO: 217)
Isovaleric acid-DTHFPCIKF)K(Palm))PRSKGWVCK-NH₂;
(SEQ ID NO: 219)
Isovaleric acid-DTHFPCIKFGP(K(Palm))SKGWVCK-NH₂;
(SEQ ID NO: 220)
Isovaleric acid-DTHFPCIKFGPRS(K(Palm))GWVCK-NH₂;
(SEQ ID NO: 221)
Isovaleric acid-DTHFPCIKFGPRSKGWVC(K(Palm))-NH₂;
(SEQ ID NO: 222)
Isovaleric acid-DTHFPCI(K(PEG3-Palm))FGPRSKGWVCK-
NH₂;
(SEQ ID NO: 223)
Isovaleric acid-DTHFPCIKF(K(PEG3-Palm))PRSKGWVCK-
NH₂;
(SEQ ID NO: 224)
Isovaleric acid-DTHFPCIKFGP(K(PEG3-Palm))SKGWVCK-
NH₂;
(SEQ ID NO: 225)
Isovaleric acid-DTHFPCIKFGPRS(K(PEG3-Palm))GWVCK-
NH₂;
(SEQ ID NO: 226)
Isovaleric acid-DTHFPCIKFGPRSKGWVC(K(PEG3-Palm))-
NH₂;
(SEQ ID NO: 176)
Isovaleric acid-DTHFPCIKFGPRSKGWVC(K(PEG8))-NH₂;
(SEQ ID NO: 241)
Isovaleric acid-DTHFPCI(K(isoGlu-Palm))FEPRSKGCK-
NH₂;
(SEQ ID NO: 242)
Isovaleric acid-DTHFPCIKF-K(isoGlu-Palm)-PRSKGCK-
NH₂;
(SEQ ID NO: 243)
Isovaleric acid-DTHFPCIKFEP(K(isoGlu-Palm))SKGCK-
NH₂;
(SEQ ID NO: 265)
Isovaleric acid-DTHFPCIKFEP(K(isoGlu-Palm))SKGWECK-
NH₂;
(SEQ ID NO: 244)
Isovaleric acid-DTHFPCIKFEPRS(K(isoGlu-Palm))GCK-
NH₂;
(SEQ ID NO: 245)
Isovaleric acid-DTHFPCIKFEPRSK(K(isoGlu-Palm))CK-
NH₂;
(SEQ ID NO: 246)
Isovaleric acid-DTHFPCIKFEPRSKGCK(K(isoGlu-Palm))-
NH₂;
(SEQ ID NO: 248)
Isovaleric acid-DTHFPCI-K(Dapa-Palm)-FEPRSKGCK-NH₂;
(SEQ ID NO: 249)
Isovaleric acid-DTHFPCIK(F(Dapa-Palm))PRSKGCK-NH₂;
(SEQ ID NO: 250)
Isovaleric acid-DTHFPCIKFEP(K(Dapa-Palm))SKGCK-NH₂;
(SEQ ID NO: 251)
Isovaleric acid-DTHFPCIKFEPRS(K(Dapa-Palm))GCK-NH₂;
(SEQ ID NO: 252)
Isovaleric acid-DTHFPCIKFEPRSK(K(Dapa-Palm))CK-NH₂;
(SEQ ID NO: 253)
Isovaleric acid-DTIFPCIKFEPRSKGC(K(Dapa-Palm))K-NH₂;
or
(SEQ ID NO: 254)
Isovaleric acid-DTHFPCIKFEPRSKGC(K(Dapa-Palm))-NH₂,
or a pharmaceutically acceptable salt thereof, wherein the peptide of Formula I′ comprises two cysteine residues linked via a disulphide bond.

8. The method of claim 1, wherein the peptide of formula I′ comprises one of the following sequences:
(SEQ ID NO: 29)
DTHFPICIFGPRSKGWVC;
(SEQ ID NO: 38)
DTHFPCIIFGPRSKGWVCK;
(SEQ ID NO: 128)
DTHFPCIIFEPRSKGWVCK;
(SEQ ID NO: 164)
DTHFPCIIFGPRSKGWACK;
(SEQ ID NO: 168)
DTHFPCIIFGPRSKGWVCKK;
(SEQ ID NO: 57
DTHFPCIIFVCHRPKGCYRRVCR;
(SEQ ID NO: 125)
DTHFPCIKFGPRSKGWVCK;
(SEQ ID NO: 173)
DTHFPCIKFKPRSKGWVCK;
(SEQ ID NO: 106)
DTHFPCIIFGPRSRGWVCK;
(SEQ ID NO: 135)
DTHFPCIKFGPKSKGWVCK;
(SEQ ID NO: 207)
DTHFPCIKFEPRSKGCK;
(SEQ ID NO: 265)
DTHFPCIKFEPKSKGWECK;
(SEQ ID NO: 245)
DTHFPCIKFEPRSKKCK;
(SEQ ID NO: 247)
DTHFPCIKFEPRSKGCKK;
(SEQ ID NO: 249)
DTHFPCIKFKPRSKGCK;
or
(SEQ ID NO: 250)
DTHFPCIKFEPKSKGCK.

9. The method of claim 1, wherein the peptide comprises one of the following sequences or structures:
(SEQ ID NO: 29)
Isovaleric acid-DTHFPICIFGPRSKGWVC-NH₂;
(SEQ ID NO: 38)
Isovaleric acid-DTHFPCIIFGPRSKGWVCK-NH₂;
(SEQ ID NO: 128)
Isovaleric acid-DTHFPCIIFEPRSKGWVCK-NH₂;
(SEQ ID NO: 164)
Isovaleric acid-DTHFPCIIFGPRSKGWACK-NH₂;
(SEQ ID NO: 168)
Isovaleric acid-DTHFPCIIFGPRSKGWVCKK-NH₂;
(SEQ ID NO: 57)
Isovaleric acid-DTHFPCIIFVCHRPKGCYRRVCR-NH₂;
(SEQ ID NO: 172)
Isovaleric acid-DTHFPCI(K(PEG8))FGPRSKGWVCK-NH₂;
(SEQ ID NO: 173)
Isovaleric acid-DTHFPCIKF(K(PEG8))PRSKGWVCK-NH₂;
(SEQ ID NO: 199)
Isovaleric acid-DTHFPICIFGPRS(K(PEG8))GWVCNH₂;
(SEQ ID NO: 200)
Isovaleric acid-DTHFPICIFGPRS(K(PEG4))GWVCNH₂;
(SEQ ID NO: 201)
Isovaleric acid-DTHFPCIIFGPRSRGWVC(K(PEG8))-NH₂;
(SEQ ID NO: 202)
Isovaleric acid-DTHFPCIIFGPRSRGWVC(K(PEG4))-NH₂;
(SEQ ID NO: 203)
Isovaleric acid-DTHFPCIIFGPRSRGWVC(K(PEG2))-NH₂;
(SEQ ID NO: 183)
Isovaleric acid-DTHFPCI(K(Palm))FGPRSKGWVCK-NH₂;
(SEQ ID NO: 217)
Isovaleric acid-DTHFPCIKF(K(Palm))PRSKGWVCK-NH₂;
(SEQ ID NO: 219)
Isovaleric acid-DTHFPCIKFGP(K(Palm))SKGWVCK-NH₂;
(SEQ ID NO: 220)
Isovaleric acid-DTHFPCIKFGPRS(K(Palm))GWVCK-NH₂;
(SEQ ID NO: 221)
Isovaleric acid-DTHFPCIKFGPRSKGWVC(K(Palm))-NH₂;
(SEQ ID NO: 222)
Isovaleric acid-DTHFPCI(K(PEG3-Palm))FGPRSKGWVCK-
NH₂;
(SEQ ID NO: 223)
Isovaleric acid-DTHFPCIKF(K(PEG3-Palm))PRSKGWVCK-
NH₂;
(SEQ ID NO: 224)
Isovaleric acid-DTHFPCIKFGP(K(PEG3-Palm))SKGWVCK-
NH₂;
(SEQ ID NO: 225)
Isovaleric acid-DTHFPCIKFGPRS(K(PEG3-Palm))GWVCK-
NH₂;
(SEQ ID NO: 226)
Isovaleric acid-DTHFPCIKFGPRSKGWVC(K(PEG3-Palm))-
NH₂;
(SEQ ID NO: 176)
Isovaleric acid-DTHFPCIKFGPRSKGWVC(K(PEG8))-NH₂;
(SEQ ID NO: 241)
Isovaleric acid-DTHFPCI(K(isoGlu-Palm))FEPRSKGCK-
NH₂;
(SEQ ID NO: 242)
Isovaleric acid-DTHFPCIKF-K(isoGlu-Palm)-PRSKGCK-
NH₂;
(SEQ ID NO: 243)
Isovaleric acid-DTHFPCIKFEP(K(isoGlu-Palm))SKGCK-
NH₂;
(SEQ ID NO: 265)
Isovaleric acid-DTHFPCIKFEP(K(isoGlu-Palm))SKGWECK-
NH₂;
(SEQ ID NO: 244)
Isovaleric acid-DTHFPCIKFEPRS(K(isoGlu-Palm))GCK-
NH₂;
(SEQ ID NO: 245)
Isovaleric acid-DTHFPCIKFEPRSK(K(isoGlu-Palm))CK-
NH₂;
(SEQ ID NO: 246)
Isovaleric acid-DTHFPCIKFEPRSKGCK(K(isoGlu-Palm))-
NH₂;
(SEQ ID NO: 248)
Isovaleric acid-DTHFPCI-K(Dapa-Palm)-FEPRSKGCK-NH₂;
(SEQ ID NO: 249)
Isovaleric acid-DTHFPCIK(F(Dapa-Palm))PRSKGCK-NH₂;
(SEQ ID NO: 250)
Isovaleric acid-DTHFPCIKFEP(K(Dapa-Palm))SKGCK-NH₂;
(SEQ ID NO: 251)
Isovaleric acid-DTHFPCIKFEPRS(K(Dapa-Palm))GCK-NH₂;
(SEQ ID NO: 252)
Isovaleric acid-DTHFPCIKFEPRSK(K(Dapa-Palm))CK-NH₂;
(SEQ ID NO: 253)
Isovaleric acid-DTIFPCIKFEPRSKGC(K(Dapa-Palm))K-NH₂;
or
(SEQ ID NO: 254)
Isovaleric acid-DTHFPCIKFEPRSKGC(K(Dapa-Palm))-NH₂.

10. The method of claim 1, wherein the disease of iron metabolism is hereditary hemochromatosis, iron hemochromatosis, HFE (human factors engineering) mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, or thalassemia.

11. The method of claim 1, wherein the disease of iron metabolism is hereditary hemochromatosis (HH).

12. The method of claim 1, wherein the disease of iron metabolism is a thalassemia, optionally thalassemia intermedia, alpha thalassemia, or β-thalassemia.

13. The method of claim 9, wherein the disease of iron metabolism is β-thalassemia.

14.-30. (canceled)

31. The method of claim 1, comprising administering the peptide in a pharmaceutical formulation comprising a pharmaceutically acceptable carrier.

32. The method of claim 31, wherein the pharmaceutical formulation is for oral, intravitreal, vaginal, nasal, topical, enteral, or parenteral administration.

33. The method of claim 1, further comprising administering the peptide or the pharmaceutically acceptable salt thereof in a dosage of about 0.0001 to about 100 mg/kg body weight per day.

34. The method of claim 1, further comprising administering the peptide or the pharmaceutically acceptable salt thereof in a dosage of about 0.0001 to about 10 mg/kg body weight per day.

35. The method of claim 1, further comprising administering the peptide or the pharmaceutically acceptable salt thereof in a dosage of about 0.0001 to about 1 mg/kg body weight per day.

36. The method of claim 35, the method further comprising administering the peptide or the pharmaceutically acceptable salt thereof once daily, twice daily, once every two, three, four, five or six days, once or twice weekly, or once or twice monthly.

37. The method of claim 1, wherein the subject is a human.
